(19) United States
(12) Patent Application Publication
(10) Pub. No.: US 2012/0301551 A1
(43) Pub. Date: Nov. 29, 2012

(54) COMPOUNDS AND COMPOSITIONS FOR TREATING NEURONAL DEATH OR NEUROLOGICAL DYSFUNCTION

(75) Inventors: Byoung Joo GWAG, Suwon-si (KR); Young Ae Lee, Suwon-si (KR); Jin Hee Shin, Seoul (KR); Sung Ig Cho, Seoul (KR); Jae Sung Noh, Anyang-si (KR); Jae Young Cho, Suwon-si (KR); Ki Won Kim, Jeonju-si (KR); Hyang Ran Lim, Seoul (KR); Jae Keun Lee, Seoul (KR); Han Yeol Byun, Seongnam-si (KR); Sun Young Ko, Suwon-si (KR); Sun Joo Son, Suwon-si (KR); Sun Mi Park, Seoul (KR)

(73) Assignee: Neurotech Pharmaceuticals Co., Ltd., Suwon-si (KR)

(21) Appl. No.: 13/531,366

(22) Filed: Jun. 22, 2012

Related U.S. Application Data
(63) Continuation of application No. 12/331,040, filed on Dec. 9, 2008, now abandoned.
Application No. 12/331,040 is a division of application No. 11/804,588, filed on May 18, 2007, now abandoned.
Application No. 11/804,588 is a continuation-in-part of application No. 11/503,379, filed on Aug. 11, 2006, now abandoned.
(60) Provisional application No. 60/780,245, filed on Mar. 8, 2006.

(30) Foreign Application Priority Data
Aug. 24, 2005 (KR) 10-2005-0078028

(51) Int. Cl. A61K 33/00; A61K 31/609; A61P 25/16; A61P 25/00; A61P 27/02; A61P 25/28; A61P 27/06; A61K 31/195

(52) U.S. Cl. 424/600; 514/166; 514/567

(57) ABSTRACT

The present invention relates to 2-hydroxy-alkylamino-benzoic acid derivatives and to a combination of cell necrosis inhibitor and lithium, process for the preparation of the derivatives or the combination, pharmaceutical formulation containing the derivatives or the combination, and use of the derivatives or the combination by either concomitant or sequential administration for improvement of treatment of neuronal death or neurological dysfunction. The derivatives and the combination of the present invention are useful for treating neurological diseases, such as amyotrophic lateral sclerosis (ALS, Lou Gehrig's disease), spinal muscular atrophy, Alzheimer's disease, Parkinson's disease, Huntington's disease, stroke, traumatic brain injury or spinal cord injury; and for treating ocular diseases such as glaucoma, diabetic retinopathy or macular degeneration.

### CROSS-REFERENCE TO RELATED APPLICATIONS

This application is a continuation of U.S. patent application Ser. No. 12/331,040 filed Dec. 9, 2008, now pending; which application is a divisional of U.S. patent application Ser. No. 11/804,588 filed May 18, 2007 and abandoned; which application is a continuation-in-part of U.S. patent application Ser. No. 11/503,379 filed Aug. 11, 2006 and abandoned; which application claims the benefit under 35 U.S.C. §119(e) of U.S. Provisional Patent Application No. 60/780,245 filed Mar. 8, 2006 and priority to South Korean Application No. 10-2005-0078028 filed Aug. 24, 2005; which applications are incorporated by reference herein in their entirety.

### BACKGROUND OF THE INVENTION

1. Field of the Invention

The present invention relates generally to 2-hydroxy-alkylamino-benzoic acid derivatives and to a combination of a cell necrosis inhibitor and lithium, process for the preparation of the derivatives or the combination, pharmaceutical formulation containing the derivatives or the combination, and use of the derivatives (or the combination by either concomitant or sequential administration) for improvement of treatment of neuronal death or neurological dysfunction. The derivatives and the combination of the present invention are useful for treating neurological diseases such as amyotrophic lateral sclerosis (ALS, Lou Gehrig's disease), spinal muscular atrophy, Alzheimer's disease, Parkinson's disease, Huntington's disease, stroke, traumatic brain injury or spinal cord injury, and ocular diseases such as glaucoma, diabetic retinopathy or macular degeneration.

2. Description of the Related Art

Neuronal death is a major neuropathological event in acute and chronic neurological diseases such as amyotrophic lateral sclerosis (ALS, Lou Gehrig's disease), spinal muscular atrophy, Alzheimer's disease, Parkinson's disease, Huntington's disease, stroke, or spinal cord injury, and ocular diseases such as glaucoma, diabetic retinopathy or macular degeneration, and can result in catastrophic dysfunction in brain, spinal cord and eye (Osborne et al., 1999; Lewen et al., 2000; Danysz et al., 2001; and Behl et al., 2002). Thus, mechanisms and interventional therapy of neuronal death have been extensively studied.

A substantial body of evidence suggests that necrosis is a dominant pattern of pathological neuronal death and can be induced by activation of various intrinsic and extrinsic death pathways including oxidative stress and excitotoxicity (Beal, 1996; Dugan & Choi, 1994). Oxidative stress is described as excess accumulation of free radicals such as reactive oxygen or nitrogen species in cells due to a mismatch between generation and elimination of free radicals. Cellular overload of free radicals can attack target molecules including DNA, proteins, and lipids, which results in cell dysfunction and degeneration. Excitotoxicity is induced by excess activation of ionotropic glutamate receptors sensitive to N-methyl-D-aspartate (NMDA) and α-amino-3-hydroxy-5-methyl-4-isoxazolepropionic acid (AMPA). Oxidative stress and excitotoxicity cause cell body swelling, scattering condensation of nuclear chromatin, and early fenestration of plasma membrane, which results in cell necrosis (Gwag et al., 1997; Nicotera et al., 1997; Won et al., 2000).

Evidence has accumulated demonstrating that oxidative stress and excitotoxicity mediate neuronal death in animal models and patients of various neurological diseases (Rao & Weiss, 2003; Waldmeier, 2003; Meldrum, 2000). It includes mitochondrial abnormalities, generation of pro-oxidants, and oxidation of DNA, protein, and lipid in Alzheimer's disease (Mecocci et al., 1994), Parkinson's disease (Dauer et al., 2003), amyotrophic lateral sclerosis (ALS, Lou Gehrig's disease) (Beal, 2001), Huntington's disease (Beal et al., 1995), stroke (Won et al., 2002), spinal cord injury (Brown et al., 1992), and ocular diseases including glaucoma, diabetic retinopathy, and macular degeneration (Takahashi et al., 2004).

Several compounds preventing oxidative stress and excitotoxicity were shown to protect neurons in animal models of ALS (Andreassen et al., 2000; Gurney et al., 1997), Alzheimer's disease (Sung et al., 2004; Miguel-Hidalgo et al., 2002), stroke (Holtzman et al., 1996; Park et al., 1988), Huntington's disease (Andreassen et al., 2001; Beister et al., 2004), spinal cord injury (Faden & Salzman, 1992; Faden et al., 1994), Parkinson's disease (Prasad et al., 1999; Rabey et al., 1992), glaucoma (Neufeld et al., 2002; Pang et al., 1999), diabetic retinopathy (Chung et al., 2005; Smith et al., 2002), and macular degeneration (Richer et al., 2004).

Several compounds preventing oxidative stress and excitotoxicity have been examined for prevention of cell death and neurological function deficit in clinical trials of stroke, Alzheimer's disease, and Parkinson's disease (Gilgun-Sherki et al., 2002). However, the clinical trials of antioxidants such as vitamin E and acetyl-L-carnitine have failed to show beneficial effects in Alzheimer's disease and Parkinson's disease (Hudson & Tabet, 2003; Thal et al., 2003; Luchsinger et al., 2003; Morens et al., 1996). Low potency and blood brain barrier permeability of the antioxidants underlie unsuccessful outcome in the clinical trials (Gilgun-Sherki et al., 2002; Molina et al., 1997). A number of NMDA antagonists have been developed and shown to reduce hypoxic-ischemic brain injury in various animal models. However, none of them have been beneficial in the clinical trials of ischemic stroke patients mainly due to the narrow therapeutic index and time window of NMDA antagonists (Labiche et al., 2004; Hoyte et al., 2004; Ikonomidou. & Turski, 2002). Thus, the therapeutic limitation of necrosis-inhibiting compounds preventing oxidative stress and excitotoxicity remains to be resolved.

Apoptosis has been coined as an additional route of pathological neuronal death. Apoptosis is accompanied by cell body shrinkage, aggregated condensation of nuclear chromatin, and fenestration of nuclear membrane with preservation of plasma membrane (Kerr et al., 1972), which differs from neuronal cell necrosis showing cell body swelling, scattering condensation of nuclear chromatin, and collapse of plasma membrane with preservation of nuclear membrane (Gwag et al., 1995; Won et al., 2000). Recently, neurotrophins that block neuronal apoptosis induce and/or potentiate neuronal cell necrosis in vitro and in vivo (Gwag & Kim, 2003; Koh et al., 1995; Won et al., 2000; Kim et al., 2002; and Barde 1994). This hints that apoptosis and necrosis may be propagated through mutually distinctive signaling pathways. Nuclear chromatin condensation, upregulation of pro-apoptotic proteins such as Bax, and activation of caspase-3, a downstream mediator of apoptosis, have been observed in human specimens of Alzheimer's disease (Kang et al., 2005; Su et al., 1997), Parkinson's disease (Hartman et al., 2000; Tatton, 2000), and ALS (Wootz et al., 2004, Biochem Biophys Res Commun., 322(1):281-6; Martin, 1999; Mu et al., 1996) and animal models of neurological diseases including Parkinson's disease (Turmel et al., 2001; Vila et al., 2001), ALS (Li et al, 2000; Gonzalez et al., 2000), stroke (Chan et al., 2004, Neurochem Res., 29(11):1943-9; Won et al., 2002; Choi, 1996), and traumatic spinal cord injury (Emery et al., 1998; Fiskum, 2000).

Anti-apoptosis drugs have been developed for the prevention of neuronal death. These include peptide inhibitors of caspases (Honig et al., 2000; Robertson et al., 2000), neurotrophic factors (Gwag & Kim, 2003; Lewin & Barde, 1996), and c-Jun N-terminal kinase (JNK) inhibitors such as CEP-1347 and CEP-11004 (Peng et al., 2004; Saporito et al., 2002). However, the therapeutic application of peptides, neurotrophic proteins, and JNK inhibitors should be compromised with transportation into brain (for example, peptides and proteins) and safety (for example, JNK inhibitors).

Recently, neuroprotective effects of lithium ion (Li⁺) have been reported in cultured neurons and in vivo (Kang et al., 2003; Chuang et al., 2002). Li⁺ is the lightest monovalent cation of the alkali metals, which was introduced into psychiatry in 1949 for the treatment of manic depressive illness and is widely used for the acute and prophylactic treatment of bipolar disorder and recurrent depression (Goodwin and Jamison, 1990). Li⁺ prevents neuronal apoptosis induced by low potassium (D'mello et al., 1994), ceramide (Centeno et al., 1998), staurosporine (Bijur et al., 2000), and beta amyloid (Ghribi et al., 2003) but does not attenuate cell necrosis-related neurotoxicity (Wie et al., Eur J. Pharmacol. 2000; 392(3):117-23). Li⁺ prevents apoptosis by inducing expression of Bcl-2, an anti-apoptosis protein, and brain-derived neurotrophic factor and activating phosphoinositide 3-kinase (PI3-K)-phospholipase Cγ pathway (Kang et al., 2003).

Accordingly, there is a need in the art for compositions and methods for treating neuronal death or neurological dysfunction. The present invention fulfills these needs and further provides other related advantages.

### BRIEF SUMMARY OF THE INVENTION

Groups of neuroprotective drugs that block neuronal cell necrosis induced by activation of NMDA receptor, free-radicals and/or zinc at submicromolar concentrations in cortical cell cultures and reduce infarct volume in animal models have been developed (See U.S. Pat. No. 6,964,982; U.S. Pat. No. 6,573,402; and U.S. Pat. No. 6,927,303, the disclosures of which are incorporated herein by reference in their entirety), and are used in the present invention.

Briefly stated, the present invention in one aspect is based on surprising effects of a combination of (a) a cell necrosis inhibitor including, but is not limited to, the neuroprotective compounds disclosed by U.S. Pat. No. 6,964,982; U.S. Pat. No. 6,573,402; and U.S. Pat. No. 6,927,303, and (b) lithium or a pharmaceutically acceptable salt thereof. The combination of the present invention is more useful in neuroprotection and improving neurological function of acute and chronic neurological diseases than treatment with either agent alone.

Therefore, the present invention in one embodiment provides a method for treating neuronal death in neurological disease or ocular disease in a human or animal, which comprises administering to the human or animal in need thereof a therapeutically effective amount of cell necrosis inhibitor and concomitantly or sequentially administering a therapeutically effective amount of lithium or a pharmaceutically acceptable salt thereof.

The present invention also provides a single unit dosage form, a pharmaceutical formulation or a kit for treating neuronal death in neurological disease or ocular disease in a human or animal, which comprises a therapeutically effective amount of cell necrosis inhibitor and a therapeutically effective amount of lithium or a pharmaceutical acceptable salt thereof.

Preferably, the present invention provides the method, the single unit dosage form, the pharmaceutical formulation, or the kit, wherein the neurological disease is any one selected from amyotrophic lateral sclerosis (ALS, Lou Gehrig's disease), Alzheimer's disease, Parkinson's disease, Huntington's disease, stroke, traumatic brain injury, and spinal cord injury.

Preferably, the present invention provides the method, the single unit dosage form, the pharmaceutical formulation, or the kit, wherein the ocular disease is any one selected from glaucoma, diabetic retinopathy and macular degeneration.

Preferably, the present invention provides the method, the single unit dosage form, the pharmaceutical formulation, or the kit, wherein the cell necrosis inhibitor is at least one selected from:

(i) a benzylaminosalicylic acid derivative of the following formula (I) or pharmaceutically acceptable salts thereof, and

(ii) a tetrafluorobenzyl derivative of the following formula (II) or pharmaceutically acceptable salts thereof:

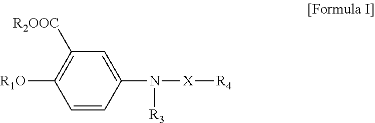

wherein,

X is CO, SO₂ or (CH₂)ₙ, wherein n is an integer from 1 to 5;

R₁ is hydrogen, alkyl or alkanoyl;

R₂ is hydrogen or alkyl;

R₃ is hydrogen or an acetoxy group; and

R₄ is a phenyl group which is unsubstituted or substituted with one or more of nitro, halogen, haloalkyl, and C₁-C₅ alkoxy;

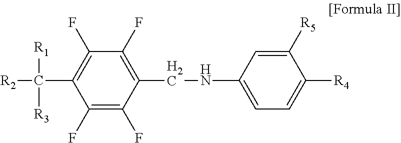

wherein,

R₁, R₂ and R₃ are independently hydrogen or halogen;

R₄ is hydroxy, alkyl, alkoxy, halogen, alkoxy substituted with halogen, alkanoyloxy or nitro; and

R₅ is carboxyl acid, ester having C₁-C₄ alkyl, carboxyamide, sulfonic acid, halogen or nitro.

The present invention provides 2-hydroxy-5-(2-(4-trifluoromethyl-phenyl)-ethylamino)-benzoic acid represented by the following chemical formula or a pharmaceutically acceptable salt thereof:

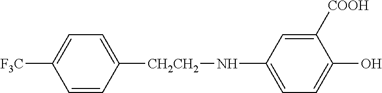

The present invention also provides a pharmaceutical composition for treating degenerative brain disease, comprising 2-hydroxy-5-(2-(4-trifluoromethyl-phenyl)-ethylamino)-benzoic acid or a pharmaceutically acceptable salt thereof.

The present invention provides a method of inhibiting production or aggregation of beta-amlyoid through the administration of a 2-hydroxy-alkylamino-benzoic acid derivative represented by the following formula or a pharmaceutically acceptable salt thereof:

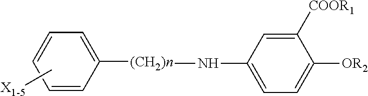

wherein,

n is an integer of 2 or 3.

R₁ is hydrogen or alkyl;

R₂ is hydrogen, alkyl or alkanoyl; and

X is independently halogen, haloalkyl or haloalkoxy.

In an embodiment of this method, the 2-hydroxy-alkylamino-benzoic acid derivative is at least one selected from 2-Hydroxy-5-(2-(4-trifluoromethyl-phenyl)-ethylamino)-benzoic acid, 5-(2-(2-Chloro-phenyl)-ethylamino)-2-hydroxy-benzoic acid, 2-Hydroxy-5-(2-(4-trifluoromethoxy-phenyl)-ethylamino)-benzoic acid, 5-(2-(3,4-Difluoro-phenyl)-ethylamino)-2-hydroxy-benzoic acid, 5-(2-(2,4-Dichloro-phenylyethylamino)-2-hydroxy-benzoic acid, 5-(2-(3,5-Bis-trifluoromethyl-phenyl)-ethylamino)-2-hydroxy-benzoic acid, 2-Hydroxy-5-(3-(4-trifluoromethyl-phenyl)-propylamino)-benzoic acid, and 5-(3-(3,4-Dichloro-phenyl)-propylamino)-2-hydroxy-benzoic acid.

In another embodiment of this method, the 2-hydroxy-alkylamino-benzoic acid derivative is 2-Hydroxy-5-(2-(4-trifluoromethyl-phenyl)-ethylamino)-benzoic acid, 5-(2-(2-Chloro-phenyl)-ethylamino)-2-hydroxy-benzoic acid, or their mixture.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

The above and other objects, features and other advantages of the present invention will be more clearly understood from the following detailed description taken in conjunction with the accompanying drawings, in which:

FIG. 1. The effects of vitamin E, 2-hydroxy-TTBA, 2-hydroxy-TPEA, and Li⁺ against free radical-mediated neuronal cell necrosis in cortical cell cultures:

A: The effects of vitamin E, 2-hydroxy-5-(2,3,5,6-tetrafluoro-4-trifluoromethyl-benzylamino)-benzoic acid (hereinafter, “2-hydroxy-TTBA”), and 2-hydroxy-5-(2-(4-trifluoromethylphenyl)ethylamino)-benzoic acid (hereinafter, “2-hydroxy-TPEA”) on Fe²⁺-induced neurotoxicity.

Mouse cortical cell cultures (DIV 11-15) were exposed to 50 μM Fe²⁺, alone or with indicated doses of 2-Hydroxy-TTBA, 2-Hydroxy-TPEA, or Vitamin E. Neuronal death was analyzed 24 hr later by measuring levels of LDH released into the bathing medium, mean±SEM (n=9-12 culture wells per condition), scaled to mean LDH efflux value 24 hr after sham wash (=0) and continuous exposure to 500 μM NMDA (=100). *, Significant difference from Fe²⁺ alone, p<0.05 using ANOVA and Student-Newman-Keuls test.

B: The effects of 2-hydroxy-TTBA and 2-hydroxy-TPEA on DL-buthionine-[S,R]-sulfoximine (a glutathione-depleting agent, hereinafter “BSO”)-induced neurotoxicity.

Mouse cortical cell cultures (DIV 11-15) were exposed to 10 mM BSO, alone or with indicated doses of 2-Hydroxy-TTBA or 2-Hydroxy-TPEA. Neuronal death was analyzed 24 hr later by measuring levels of LDH released into the bathing medium, mean±SEM (n=9-12 culture wells per condition). *, Significant difference from BSO alone, p<0.05 using ANOVA and Student-Newman-Keuls test.

C: Li⁺ does not attenuate free radical neurotoxicity.

Mouse cortical cell cultures (DIV 11-15) were exposed to 50 μM Fe²⁺ or 10 mM BSO, alone or with inclusion of 5 mM Li⁺. Neuronal death was analyzed 24 hr later by measuring levels of LDH released into the bathing medium, mean±SEM (n=9-12 culture wells per condition).

FIG. 2. The effects of vitamin E, 2-hydroxy-TTBA, 2-hydroxy-TPEA, and Li⁺ against neuronal cell apoptosis in cortical cell cultures:

A: The neuroprotective effects of Li⁺ against calyculin A or cyclosporine A-induced neuronal apoptosis.

Mouse cortical cell cultures (DIV 10-12) were exposed to 20 μM cyclosporine A or 10 nM calculin A, alone or with inclusion of 0.3-30 mM Li. Neuronal death was analyzed 24-28 hr later by measuring levels of LDH released into the bathing medium, mean±SEM (n=9-12 culture wells per condition).

B: Vitamin E, 2-hydroxy-TTBA, and 2-hydroxy-TPEA do not attenuate neuronal cell apoptosis.

Mouse cortical cell cultures (DIV 10-12) were exposed to 20 μM cyclosporine A, alone or with 100 μM Vitamin E, 1 μM 2-hydroxy-TTBA, or 1 μM 2-hydroxy-TPEA. Neuronal death was analyzed 24 hr later by measuring levels of LDH released into the bathing medium, mean±SEM (n=9-12 culture wells per condition).

FIG. 3. Analysis of oxidative stress and neuronal death in the lumbar spinal cord from ALS transgenic mice (G93AA):

A: The fluorescent photomicrographs of the lumbar spinal cord section immunolabeled with nitrotyrosine antibody (green, top panel) or double-labeled (bottom panel) with MitoTracker CM-H2XRos (red) and NeuN antibody (neuronal marker, green) in wild type (a,c) or ALS transgenic mice (b,d) at ages of 8 week. Arrows indicate motor neurons.

B: The fluorescence intensity of nitrotyrosine was analyzed in the ventral motor neurons at ages of 4 to 14 weeks, mean±SEM (n=25 sections from five mice per each group). *Significant difference between wild type and ALS transgenic mice at the same age, using Independent-Samples t-test.

C: Degeneration of the spinal motor neurons from ALS transgenic mice.

The number of the viable motor neurons in the lumbar ventral horn was analyzed after staining with cresyl violet at indicated points of age, mean±SEM (n=5 mice per each group).

FIG. 4. Activation of Fas-mediated apoptosis pathways in ALS transgenic mice:

A: Western blot analysis showing expression of Fas, FADD, and actin in the lumbar segment from wild type [Tg(−)] or ALS transgenic mice [Tg(+)] at indicated ages (top panel). Bottom panel shows interaction of Fas and FADD using Western blot analysis of FADD antibody following immunoprecipitation with Fas antibody in the same samples above.

B: Bright-field photomicrographs of the spinal motor neurons taken after immunolabeling with Fas antibody from Tg(−) (a) or Tg(+) (b) at age of 12 weeks.

C: Western blot analysis showing expression of caspase 8, caspase 3, and actin in the lumbar segment from Tg(−) or Tg(+) at indicated points of age.

D: Fluorescence photomicrographs of the lumbar ventral sections taken after immunolabeling with an antibody for cleaved caspase 3 from Tg(−) (a) or Tg(+) (b) at age of 12 weeks.

FIG. 5. Oxidative stress and apoptosis in the spinal motor neurons from ALS transgenic mice: effects of 2-hydroxy TTBA and Li⁺:

A: 2-hydroxy TTBA, but not Li⁺, prevents oxidative stress. Mouse cortical cell cultures (DIV 11-15) were exposed to 30 μM Fe²⁺ or 10 mM BSO, alone or in the presence of 1 μM 2-hydroxy TTBA, 10-100 μM vitamin E, or 5 mM Li⁺. Neuronal death was analyzed 24 h later by measuring LDH efflux in the bathing media (mean±SEM, n=12). *, Significant difference from the relevant control (Fe²⁺ or BSO alone), p<0.05 using ANOVA and Student-Newman-Keuls test.

B & C: Li⁺, but not 2-hydroxy TTBA, prevents apoptosis. (B) Neuron-rich cortical cell cultures (DIV 7) were deprived of serum, alone or with addition of 100 μM zVADfmk, 1 μM 2-hydroxy TTBA, or 5 mM Li⁺. Neuronal death was analyzed 24 hr later by counting viable neurons excluding trypan (mean±SEM, n=4). *, Significant difference from the relevant control (serum deprivation alone), p<0.05 using ANOVA and Student-Neuman-Keuls test. (C) Western blot analysis of FADD antibody following immunoprecipitation with Fas antibody in the same samples above.

D: Fluorescent photomicrographs of the lumbar ventral section immunolabeled with nitrotyrosine antibody from the wild type (a), or ALS transgenic mice treated with vehicle (b), or 2-hydroxy TTBA for 2 weeks starting from at age of 8 weeks (c). Arrows indicate motor neurons (top panel). Levels of nitrotyrosine were quantitated, mean±SEM (n=15 sections from 3 mice per each condition (bottom panel). p<0.05 using ANOVA and Student-Newman-Keuls test.

E: Same as D except measurement of the fluorescence intensity of oxidized MT red CM-H2XRos. p<0.05 using ANOVA and Student-Newman-Keuls test.

F: Western blot analysis of Fas, FADD, cleaved caspase-8, cleaved caspase-3, and actin in the lumbar segment from the wild type [Tg(−)] or Tg(+) treated with saline, 2-hydroxy TTBA, or Li⁺ for 4 weeks starting from age of 8 weeks.

FIG. 6. Co-administration of 2-hydroxy TTBA and Li⁺ synergistically improves motor function in ALS transgenic mice:

Animals were daily fed with 2-hydroxy TTBA (30 mg/kg/d), 0.2% lithium carbonate (200 mg/kg/d, Li), or a combination of both (2-hydroxy TTBA+Li) from 8 weeks of age in diet. Body weight (A), extension reflex (B), PaGE test (C), and Rotarod test (D) were analyzed at the indicated points of age, mean±SEM (n=13 per each group)*, p<0.05 compared to the vehicle; #, p<0.05 between 2-hydroxy TTBA (or Li) alone and combination of 2-hydroxy TTBA and Li.

FIG. 7. Co-administration of 2-hydroxy TTBA and Lithium synergistically delays onset of motor function deficit, survival, and degeneration of the motor neurons in ALS mice:

Animals were daily fed with 2-hydroxy TTBA (30 mg/kg/d), 0.2% lithium carbonate (Li), or a combination of both (2-hydroxy TTBA+Li) from 8 weeks of age in diet.

A & B: Cumulative probability of onset of motor function deficits (A) and cumulative probability of survival (B) in ALS transgenic mice.

C & D: (C) Bright-field photomicrographs of cresyl violet-stained ventral horn sections from the wild type (a), or G93AA transgenic mice treated with vehicle (b), or with a combination of 2-hydroxy TTBA+Li (c) at 16 weeks of age. (D) The number of viable motor neurons in the lumbar ventral horn was analyzed at 16 weeks of age, mean±SEM (n=20 sections from four mice per each group) *, p<0.01 compared to the vehicle; #, p<0.01 between 2-hydroxy TTBA (or Li) alone and combination of 2-hydroxy TTBA and Li, using ANOVA and Student-Newman-Keuls test.

FIG. 8 shows a suppressing effect of chemical 01 on reactive oxygen caused by FeCl₂ in cerebral cortical neuron.

FIGS. 9A-C show concentration-dependently suppressing effects of chemical 01 on inflammatory cytokines (IL-1β, IL-6 and TNF-alpha, respectively) increased by LPS in BV2 cell lines.

FIG. 10 is results showing degrees of the gastric mucous membrane damage. Test samples were orally administered in a different dose. FIG. 10 shows that chemical 01 did not cause damage of the gastric mucous membrane, which means that chemical 01 is safe. Aspirin was used as control.

FIG. 11A shows quantified amyloid plaque produced in brain of 17 month-old Tg2576 dementia mouse. Thioflavin-S pigment was used for staining. Chemical 01 shows the result of mouse having administration of chow with 25 mg/kg/day of chemical 01 for 8 months (from 9 to 17 months).

FIGS. 11B-E shows that Aβ₄₂ or Aβ₄₀ protein levels were decreased by administration of chemical 01 in brain of 17 month-old Tg2576 dementia mouse. In each figure, chemical 01 shows the result of mouse having administration of chow with 25 mg/kg/day of chemical 01 for 8 months (from 9 to 17 months). FIGS. 11B-E show the decreases of soluble Aβ₄₂, insoluble Aβ₄₂, soluble Aβ₄₀ and insoluble Aβ₄₀ protein level, respectively.

FIG. 11F shows the results of Morris water maze test performed with 14 month-old normal mouse, 14 month-old Tg2576 dementia mouse fed with only chow, and 14 month-old Tg2576 mouse having administration of chow with 25 mg/kg/day of chemical 01 for 5 months (from 9 months). The latency to find the platform was recorded for evaluating the therapeutic efficacy.

FIG. 11G shows the results of Elevated plus maze test performed with 14 month-old normal mouse, 14 month-old Tg2576 dementia mouse fed with only chow, and 14 month-old Tg2576 mouse having administration of chow with 25 mg/kg/day of chemical 01 for 5 months (from 9 months). The time spent in the open arm was recorded in the elevated plus maze.

FIGS. 12A-D show the decreases of the levels of Aβ₄₂ or Aβ₄₀ proteins in brain of APP/PS1 dementia mouse. 17.5 month-old APP/PS1 dementia mouse fed with only chow, or 17.5 month-old APP/PS1 dementia mouse having chronic administration of chow with 25 mg/kg/day of chemical 01 or 62.5 mg/kg/day of ibuprofen for 14.5 months (from 3 to 17.5 months) were evaluated.

FIGS. 12A-D show the decreases of the levels of soluble Aβ₄₂, insoluble Aβ₄₂, soluble Aβ₄₀ and insoluble Aβ₄₀, respectively, in brains of dementia mouse by administration of chemical 01.

FIG. 12E is the results of Morris water maze test performed with 17.5 month-old normal mouse, 17.5 month-old APP/PS1 dementia mouse fed with only chow, and 17.5 month-old APP/PS1 mouse having chronic administration of chow with 25 mg/kg/day of chemical 01 or 62.5 mg/kg/day of ibuprofen for 14.5 months (from 3 to 17.5 months). The latency to find the platform was recorded for evaluating the therapeutic efficacy.

FIG. 12F is the results of Elevated plus maze test performed with 17.5 month-old normal mouse, 17.5 month-old APP/PS1 dementia mouse fed with only chow, and 17.5 month-old APP/PS1 mouse having chronic administration of chow with 25 mg/kg/day of chemical 01 or 62.5 mg/kg/day of ibuprofen for 14.5 months (from 3 to 17.5 months). The time spent in the open arm was recorded in the elevated plus maze.

FIG. 12G is the results of Open field activity test performed with 17.5 month-old normal mouse, 17.5 month-old APP/PS1 dementia mouse fed with only chow, and 17.5 month-old APP/PS1 mouse having chronic administration of chow with 25 mg/kg/day of chemical 01 or 62.5 mg/kg/day of ibuprofen for 14.5 months (from 3 to 17.5 months). It was recorded how close each mouse approaches the open field.

FIGS. 13A-B show improvements of motor function in G93A ALS animal model (G93A mouse) by administration of chemical 01. FIG. 13A is the result of Rotarod test for evaluating general walking and degree of symmetrical myokinesis. FIG. 13B is the result of PaGE test for evaluating muscular force of limb.

FIGS. 13C-D show effects delaying onset and extending survival in G93A mouse by administration of chemical 01. FIG. 13C is the result showing the onset of each group calculated by probability. FIG. 13D is the result showing the survival rate of G93A mice in each group, calculated by probability.

FIG. 13E shows the suppressing effect of chemical 01 on oxidative toxicity in G93A mouse. The fluorescence intensity was quantified by immunostaining using nitrotyrosine.

FIG. 13F shows the suppressing effect of chemical 01 on inflammation in G93A mouse. The results were immunostained with TOMATO Lectin.

A: Normal mouse

B: G93A mouse

C: G93A mouse having administration of 5 mg/kg/day chemical 01

D: G93A mouse having administration of 20 mg/kg/day chemical 01

FIG. 13G shows the suppressing effect of chemical 01 on inflammation in G93A mouse. RT-PCR (Reverse Transcription-Polymerase Chain Reaction) was performed, and mRNA levels of inflammatory cytokines were evaluated.

FIG. 14A shows the protecting effect of chemical 01 on neurotoxicity caused by 50 uM MPP+ in cerebral cortical cell culture, cell culture model of Parkinson's disease.

FIG. 14B shows the protecting effect of chemical 01 on dopaminergic neurodegeneration caused by LPS in mesencephalic culture, cell culture model of Parkinson's disease.

FIG. 14C shows the suppressing effect of chemical 01 on NO produced by LPS in mesencephalic culture.

FIG. 14D shows the suppressing effect of chemical 01 on TNF-α produced by LPS in mesencephalic culture.

FIG. 14E shows the suppressing effect of chemical 01 on inflammation in animal model of Parkinson's disease. Results were immunostained with CD11b.

A: Mouse having administration of MPTP

B: Mouse having administration of 50 mg/kg/day chemical 01

FIG. 15 is the results of single dose toxicity testing of chemical 01, chemical 27, chemical 07, chemical 04 and chemical 42.

FIG. 16. The effect of chemical_{—}01 in Tg2576 transgenic mice

A: The amyloid plaque density was analyzed by fluorescent thioflavin-S staining in the brain sections from 17 month-old Tg2576 transgenic mice (control), chronic administration of chow with 25 mg/kg/day of chemical_{—}01 for 8 months (from 9 to 17 months), mean±SEM (n=2 animals). *, Significant difference from control, p<0.05 using ANOVA and Student-Newman-Keuls test.

B-C: The SDS-soluble Aβ₄₂ levels (B) or SDS-insoluble Aβ₄₂ levels (C) were analyzed by colorimetric sandwich ELISA kit in the brain homogenates from 17 month-old Tg2576 transgenic mice (control) and chronic administration of chow with 25 mg/kg/day of chemical_{—}01 for 8 months (from 9 to 17 months), mean±SEM (n=2 animals). *, Significant difference from control p<0.05 using ANOVA and Student-Newman-Keuls test.

D-E: The SDS-soluble Aβ₄₀ levels (D) or SDS-insoluble Aβ₄₀ levels (E) were analyzed by colorimetric sandwich ELISA kit in the brain homogenates from 17 month-old Tg2576 transgenic mice (control) and chronic administration of chow with 25 mg/kg/day of chemical_{—}01 for 8 months (from 9 to 17 months), mean±SEM (n=2 animals). *, Significant difference from control p<0.05 using ANOVA and Student-Newman-Keuls test.

F: The cognitive function was analyzed in the Morris water maze from 14 month-old Tg2576 transgenic mice(control) and chronic administration of chow with 25 mg/kg/day of chemical_{—}01 for 5 months (from 9 to 14 months), mean±SEM (n=2 animals). * and **, Significant difference from control p<0.05 and <0.01, respectively, using ANOVA and Student-Newman-Keuls test.

G: The degree of anxiety was analyzed in the elevated plus maze from 14 month-old Tg2576 transgenic mice(control) and chronic administration of chow with 25 mg/kg/day of chemical_{—}01 for 5 months (from 9 to 14 months), mean±SEM (n=2 animals). *, Significant difference from control p<0.05 using ANOVA and Student-Newman-Keuls test.

FIG. 17. The effect of chemical_{—}01 in APP_{SWE}/PS1_{deltaE9} double transgenic mice

A-B: The SDS-soluble Aβ₄₂ levels (A) or SDS-insoluble Aβ₄₂ levels (B) were analyzed by colorimetric sandwich ELISA kit in the brain homogenates from 17.5 month-old APP_{SWE}/PS1_{deltaE9} double transgenic mice (control) and chronic administration of chow with 25 mg/kg/day of chemical_{—}01 or 62.5 mg/kg/d of ibuprofen for 14.5 months (from 3 to 17.5 months), mean±SEM (n=3-5 animals). *, Significant difference from control p<0.05 using ANOVA and Student-Newman-Keuls test.

C-D: The SDS-soluble Aβ₄₀ levels (C) or SDS-insoluble Aβ₄₀ levels (D) were analyzed by colorimetric sandwich ELISA kit in the brain homogenates from 17.5 month-old APP_{SWE}/PS1_{deltaE9} double transgenic mice (control) and chronic administration of chow with 25 mg/kg/day of chemical_{—}01 or 62.5 mg/kg/d of ibuprofen for 14.5 months (from 3 to 17.5 months), mean±SEM (n=3-5 animals). *, Significant difference from control p<0.05 using ANOVA and Student-Newman-Keuls test.

E: The cognitive function was analyzed in the Morris water maze from 17.5 month-old APP_{SWE}/PS1_{deltaE9} double transgenic mice (control) and chronic administration of chow with 25 mg/kg/day of chemical_{—}01 or 62.5 mg/kg/d of ibuprofen for 14.5 months (from 3 to 17.5 months), mean±SEM (n=3-5 animals). *, Significant difference from control p<0.05 using ANOVA and Student-Newman-Keuls test.

F: The degree of anxiety was analyzed in the elevated plus maze from 17.5 month-old APP_{SWE}/PS1_{deltaE9} double transgenic mice (control) and chronic administration of chow with 25 mg/kg/day of chemical_{—}01 or 62.5 mg/kg/d of ibuprofen for 14.5 months (from 3 to 17.5 months), mean±SEM (n=3-5 animals). *, Significant difference from control p<0.05 using ANOVA and Student-Newman-Keuls test.

G: The open field activity, measured traveled distance in open field, was analyzed from the 17.5 month-old APP_{SWE}/PS1_{deltaE9} double transgenic mice (control) and chronic administration of chow with 25 mg/kg/day of chemical_{—}01 or 62.5 mg/kg/d of ibuprofen for 14.5 months (from 3 to 17.5 months), mean±SEM (n=3-5 animals). *, Significant difference from control p<0.05 using ANOVA and Student-Newman-Keuls test.

### DETAILED DESCRIPTION OF THE INVENTION

The present invention in one aspect relates to a combination of at least one cell necrosis inhibitor; and lithium or a pharmaceutically acceptable salt thereof. The present invention also relates to a method for improving the treatment of neuronal death in neurological disease or ocular disease, a single unit dosage form, a pharmaceutical formulation or a kit using the combination.

Therefore, the present invention in one embodiment provides a method for treating neuronal death in neurological disease or ocular disease in a human or animal, which comprises administering to the human or animal in need thereof a therapeutically effective amount of a cell necrosis inhibitor and concomitantly or sequentially administering a therapeutically effective amount of lithium or a pharmaceutically acceptable salt thereof. As used herein, the term “treating” includes “preventing.”

Examples of neurological diseases that may be treated with the combination of the present invention include, but are not limited to, amyotrophic lateral sclerosis (ALS, Lou Gehrig's disease), Alzheimer's disease, Parkinson's disease, Huntington's disease, stroke, traumatic brain injury, and spinal cord injury. Examples of ocular diseases that may be treated with the combination of the present invention include, but are not limited to, glaucoma, diabetic retinopathy and macular degeneration. Relations between the concrete diseases mentioned above and the combination of the present invention are described below in more detail.

The combination of the present invention comprises a cell necrosis inhibitor and, preferably, the cell necrosis inhibitor is, but is not limited to, at least one selected from:

(i) a benzylaminosalicylic acid derivative of the following formula (I) or pharmaceutically acceptable salts thereof, and

(ii) a tetrafluorobenzyl derivative of the following formula (II) or pharmaceutically acceptable salts thereof:

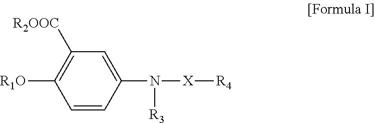

wherein,

X is CO, SO₂ or (CH₂)ₙ, wherein n is an integer from 1 to 5;

R₁ is hydrogen, alkyl or alkanoyl;

R₂ is hydrogen or alkyl;

R₃ is hydrogen or an acetoxy group; and

R₄ is a phenyl group which is unsubstituted or substituted with one or more of nitro, halogen, haloalkyl, and C₁-C₅ alkoxy;

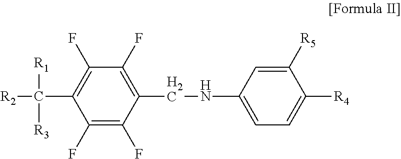

wherein,

R₁, R₂ and R₃ are independently hydrogen or halogen;

R₄ is hydroxy, alkyl, alkoxy, halogen, alkoxy substituted with halogen, alkanoyloxy or nitro; and

R₅ is carboxyl acid, ester having C₁-C₄ alkyl, carboxyamide, sulfonic acid, halogen or nitro.

In formula I and II, alkyl is C₁-C₄ alkyl, and more preferably C₁-C₂ alkyl. Alkyl described above includes, but is not limited to, methyl, ethyl, propyl, isopropyl, n-butyl, sec-butyl, and tert-butyl. Alkoxy is C₁-C₄alkoxy, and more preferably C₁-C₂ alkoxy. Alkoxy described above includes, but is not limited to, methoxy, ethoxy, and propanoxy. Halogen includes, but is not limited to, fluoride, chloride, bromide, and iodide. Alkanoyl is C₂-C₁₀ alkanoyl, and more preferably C₃-C₅ alkanoyl. Alkanoyl described above includes, but is not limited to, ethanoyl, propanoyl, and cyclohexanecarbonyl. Alkanoyloxy is C₂-C₁₀ alkanoyloxy, and more preferably C₃-C₅ alkanoyloxy. Alkanoyloxy described above includes, but is not limited to, ethanoyloxy, propanoyloxy, and cyclohexanecarbonyloxy.

The benzylaminosalicylic acid derivatives and tetrafluorobenzyl derivatives are more preferable than other cell necrosis inhibitors when considering their efficacy and synergic effect with lithium. These cell necrosis inhibitors (See U.S. Pat. No. 6,964,982; U.S. Pat. No. 6,573,402; and U.S. Pat. No. 6,927,303, the disclosures of which are incorporated herein by reference in their entirety) in nanomolar range block completely cell-necrosis-related neurotoxicity and confirm neuroprotective effects in animal models of stroke, spinal cord injury or ALS.

After considering safety and therapeutic efficiency including neuroprotective effect of cell necrosis inhibitors, and combination synergy with lithium, examples of the benzylaminosalicylic acid derivatives include, but are not limited to,
5-benzylaminosalicylic acid (BAS),
5-(4-nitrobenzyl)aminosalicylic acid (NBAS),
(5-(4-chlorobenzyl)aminosalicylic acid (CBAS),
(5-(4-trifluoro-methylbenzyl)aminosalicylic acid (TBAS),
(5-(4-fluorobenzyl)aminosalicylic acid (FBAS),
5-(4-methoxybenzyl)aminosalicylic acid (MBAS),
5-(pentafluoro-benzyl)aminosalicylic acid (PBAS),
5-(4-nitrobenzyl)amino-2-hydroxy ethylbenzoate,
5-(4-nitrobenzyl)-N-acetylamino-2-hydroxy ethylbenzoate,
5-(4-nitrobenzyl)-N-acetylamino-2-acetoxy ethylbenzoate,
5-(4-nitrobenzoyl)aminosalicylic acid,
5-(4-nitrobenzenesulfonyl)aminosalicylic acid,
5-[2-(4-nitrophenyl)-ethyl]aminosalicylic acid (NPAA),
5-[3-(4-nitrophenyl)-n-propyl]aminosalicylic acid (NPPAA),
2-hydroxy-5-(2-(4-trifluoromethyl-phenyl)ethylamino)-benzoic acid (2-hydroxy-TPEA), and

pharmaceutically acceptable salts thereof;

and examples of the tetrafluorobenzyl derivatives include, but are not limited to,
2-hydroxy-5-(2,3,5,6-tetrafluoro-4-trifluoromethyl-benzylamino)-benzoic acid (2-Hydroxy-TTBA),
2-nitro-5-(2,3,5,6-tetrafluoro-4-trifluoromethyl-benzylamino)benzoic acid,
2-chloro-5-(2,3,5,6-tetrafluoro-4-trifluoromethylbenzylamino)benzoic acid,
2-bromo-5-(2,3,5,6-tetrafluoro-4-trifluoromethyl-benzylamino)benzoic acid,
2-hydroxy-5-(2,3,5,6-tetrafluoro-4-methylbenzylamino)benzoic acid,
2-methyl-5-(2,3,5,6-tetrafluoro-4-trifluoromethyl-benzylamino)benzoic acid,
2-methoxy-5-(2,3,5,6-tetrafluoro-4-trifluoromethylbenzylamino)benzoic acid,
5-(2,3,5,6-tetrafluoro-4-trifluoromethyl-benzylamino)-2-trifluoromethoxy benzoic acid,
2-nitro-4-(2,3,5,6-tetrafluoro-4-trifluoromethylbenzylamino)phenol,
2-chloro-4-(2,3,5,6-tetrafluoro-4-trifluoromethylbenzylamino)phenol,
2-hydroxy-5-(2,3,5,6-tetrafluoro-4-trifluoromethyl-benzylamino)benzamide,
2-hydroxy-5-(2,3,5,6-tetrafluoro-4-trifluoromethylbenzylamino)benzenesulfonic acid,
methyl 2-hydroxy-5-(2,3,5,6-tetrafluoro-4-trifluoromethyl-benzylamino)benzoate,
2-ethanoyloxy-5-(2,3,5,6-tetrafluoro-4-trifluoromethyl-benzylamino)benzoic acid,
2-propanoyloxy-5-(2,3,5,6-tetrafluoro-4-trifluoromethylbenzylamino)benzoic acid,
2-cyclohexanecarbonyloxy-5-(2,3,5,6-tetrafluoro-4-trifluoromethylbenzylamino)benzoic acid, and

pharmaceutically acceptable salts thereof.

The cell necrosis inhibitor compounds of the present invention can exist as a pharmaceutically acceptable salt. Pharmaceutically acceptable acid addition salts of the present compounds can be formed of the compound itself, or of any of its esters, and include the pharmaceutically acceptable salts which are often used in pharmaceutical chemistry. For example, salts may be formed with organic or inorganic acids. Suitable organic acids include maleic, fumaric, benzoic, ascorbic, succinic, methanesulfonic, benzenesulfonic, toluenesulfonic, acetic, oxalic, trifluoroacetic, propionic, tartaric, salicylic, citric, gluconic, lactic, mandelic, cinnamic, aspartic, stearic, palmitic, formic, glycolic, glutamic, and benzenesulfonic acids. Suitable inorganic acids include hydrochloric, hydrobromic, sulfuric, phosphoric, and nitric acids. Additional salts include chloride, bromide, iodide, bisulfate, acid phosphate, isonicotinate, lactate, acid citrate, oleate, tannate, pantothenate, bitartrate, gentisinate, gluconate, glucaronate, saccharate, ethanesulfonate, p-toluenesulfonate, and pamoate (i.e., 1,1′-methylene-bis-(2-hydroxy-3-naphthoate)) salts. The term “pharmaceutically acceptable salt” is intended to encompass any and all acceptable salt forms.

Pharmaceutically acceptable salts can be formed by conventional and known techniques, such as by reacting an inhibitor compound of this invention with a suitable acid as disclosed above. Such salts are typically formed in high yields at moderate temperatures, and often are prepared by merely isolating the compound from a suitable acidic wash in the final step of the synthesis. The salt-forming acid may be dissolved in an appropriate organic solvent, or aqueous organic solvent, such as an alkanol, ketone or ester. On the other hand, if the compound of the present invention is desired in the free base form, it may be isolated from a basic final wash step, according to known techniques. For example, a typical technique for preparing a hydrochloride salt is to dissolve the free base in a suitable solvent, and dry the solution thoroughly, as over molecular sieves, before bubbling hydrogen chloride gas through it.

In addition, some of the cell necrosis inhibitor compounds of the present invention may be in a hydrated form, and may exist as solvated or unsolvated form. A part of compounds exist as crystal form or amorphous form, and any physical form is included in the scope of the present invention.

The cell necrosis inhibitors of the present invention may contain one or more asymmetric carbon atoms and therefore exist in two or more stereoisomeric forms. The present invention includes these individual stereoisomers of the inhibitors of the present invention.

The combination of the present invention comprises lithium or a pharmaceutically acceptable salt thereof and, preferably, the salt includes, but is not limited to, lithium carbonate, lithium chloride, lithium bromide, lithium acetate, lithium citrate, lithium succinate, lithium acetylsalicylate, lithium benzoate, lithium bitartrate, lithium nitrate, lithium selenate, lithium sulphate, lithium aspartate, lithium gluconate and lithium thenoate.

In addition, the combination of the present invention may comprise a lithium salt of the benzylaminosalicylic acid derivative or the tetrafluorobenzyl derivative.

Further, the present invention provides a single unit dosage form, a pharmaceutical formulation or a kit comprising the cell necrosis inhibitor and lithium or its salt. A kit may also include instructions.

The combination of the present invention may be produced in one pharmaceutical formulation comprising both the cell necrosis inhibitor and lithium (or its salt) or in two different pharmaceutical formulations, one for the cell necrosis inhibitor and one for the lithium. The pharmaceutical formulation may be in the form of tablets, capsules, powders, mixtures, solutions, suspensions or other suitable pharmaceutical formulation forms. The pharmaceutical formulation of the present invention may comprise a pharmaceutically acceptable excipient for easiness of manufacturing, and appearance and stability of the formulation.

Routes of administration of the combination of the present invention include, but are not limited to, oral, topical, subcutaneous, transdermal, subdermal, intra-muscular, intra-peritoneal, intravesical, intra-articular, intra-arterial, intra-venous, intra-dermal, intra-cranial, intra-lesional, intra-tumoral, intra-ocular, intra-pulmonary, intra-spinal, intraprostatic, placement within cavities of the body, nasal inhalation, pulmonary inhalation, impression into skin and electrocorporation.

To produce pharmaceutical formulations of the combination of the invention in the form of dosage units for oral application, the selected compounds may be mixed with a solid excipient, for example, a diluent such as lactose, mannitol, microcrystalline cellulose and corn starch; a binder such as gelatin and polyvinylpyrrolidone; a disintegrator such as sodium starch glycolate and cross-carmellose sodium; a lubricant such as magnesium stearate, wax and so on; and the like, and then compressed into tablets. If coated tablets are required, the tablet cores prepared above may be coated with a coating material such as gelatin, hydroxypropylmethylcellulose and so on.

For the formulation of soft gelatin capsules, the two active substances may be admixed with, for example, a vegetable oil or poly-ethylene glycol. Hard gelatin capsules may contain granules of the two active substances using a method well known to those skilled in the art.

Liquid formulation for oral application may be in the form of syrups, solutions or suspensions, and such liquid formulations may contain coloring agents, flavoring agents, sugar, stabilizers, surfactants, thickening agent or other excipients known to those skilled in the art.

Solutions for parenteral applications by injection can be prepared in an aqueous solution of a water-soluble pharmaceutically acceptable salt of the two active ingredients, preferably in a concentration of from about 0.1% to about 20% by weight. These solutions may also contain stabilizing agents, buffering agents and/or pH-adjusting agents, and may be conveniently prepared by conventional methods.

Further, the present invention provides a kit comprising the combination of the cell necrosis inhibitor and lithium or a pharmaceutically acceptable salt thereof, optionally with instructions for use.

The particular therapeutic agent administered, the amount per dose, the dose schedule and the route of administration should be decided by the practitioner using methods known to those skilled in the art and will depend on the type of neurological disease or ocular disease, the severity of the diseases, the location of the diseases and other clinical factors such as the size, weight and physical condition of the recipient. In addition, in vitro assays may optionally be employed to help identify optimal ranges for sequence administration. For the purpose of this invention, daily dosage of the cell necrosis inhibitor may be in the range of about 0.1 mg-100 g/kg bodyweight, preferably about 0.5 mg-10 g/kg bodyweight, more preferably about 1 mg-1 g/kg bodyweight. Also, daily dosage of lithium for the adult human may generally be in the range of 1-2000 mg, preferably 20-600 mg, more preferably 50-600 mg/kg bodyweight (See U.S. Pat. No. 4,753,964, the disclosure of which is incorporated herein by reference in its entirety). As occasion demands, the combination of the present invention can be administered in small doses 1 to 4 times a day over variable times from weeks to months.

The present invention also provides 2-hydroxy-5-(2-(4-trifluoromethyl-phenyl)ethylamino)-benzoic acid represented by the following formula or a pharmaceutically acceptable salt useful for treating degenerative brain disease:

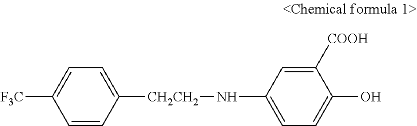

The term “pharmaceutically acceptable salt” of the present invention means salts produced by non-toxic or little toxic base. Base addition salts of the compound of the present invention can be made by reacting the free base of the compound with enough amount of desirable base and adequate inert solvent. Pharmaceutically acceptable base addition salt includes, but is not limited to, lithium, sodium, potassium, calcium, ammonium, magnesium or salt made by organic amino.

In addition, the compound of the present invention may be hydrated form, and may exist as solvated or unsolvated form. The compound exists as crystal form or amorphous form, and any physical form is included in the scope of the present invention.

The present invention also provides a pharmaceutical composition comprising the compound or its pharmaceutically acceptable salt; and pharmaceutically acceptable carrier or diluant (including excipient or additive). The compound or its pharmaceutically acceptable salt of the present invention may be administered alone or with any convenient carrier, diluent, etc., and a formulation for administration may be single-dose unit or multiple-dose unit.

A pharmaceutical composition of the present invention may be formulated in a solid or liquid form. The solid formulation includes, but is not limited to, a powder, a granule, a tablet, a capsule, a suppository, etc. Also, the solid formulation may further include, but is not limited to, a diluent, a flavoring agent, a binder, a preservative, a disintegrating agent, a lubricant, a filler, etc. The liquid formulation includes, but is not limited to, a solution such as water solution and propylene glycol solution, a suspension, an emulsion, etc., and may be prepared by adding suitable additives such as a coloring agent, a flavoring agent, a stabilizer, a thickener, etc.

A composition of the present invention may be administered in forms of, but not limited to, oral formulation, injectable formulation (for example, intramuscular, intraperitoneal, intravenous, infusion, subcutaneous, implant), inhalable, intranasal, vaginal, rectal, sublingual, transdermal, topical, etc., depending on the disorders to be treated and the patient's conditions. The composition of the present invention may be formulated in a suitable dosage unit comprising a pharmaceutically acceptable and non-toxic carrier, additive and/or vehicle, which all are generally used in the art, depending on the routes to be administered.

The present invention also provides a method for treating degenerative brain disease, comprising administering to a subject in need thereof a therapeutically effective amount of the compound or a pharmaceutically acceptable salt. In more detail, the compound or its salt can be used for treating Alzheimer's disease, Parkinson's disease, Lou Gehrig's disease, Huntington's disease, etc.

For treating degenerative brain disease, the compound of the present invention may be administered daily at a dose of approximately 0.01 mg/kg to approximately 100 g/kg, preferably approximately 0.1 mg/kg to approximately 10 g/kg. However, the dosage may be varied according to the patient's conditions (age, sex, body weight, etc.), the severity of patients in need thereof, the used effective components, diets, etc. The compound of the present invention may be administered once a day or several times a day in divided doses, if necessary.

2-hydroxy-5-(2-(4-trifluoromethyl-phenyl)-ethylamino)-benzoic acid and its pharmaceutically acceptable salt can be prepared by the following reaction scheme. However, the following reaction methods are offered by way of illustration and are not intended to limit the scope of the invention.

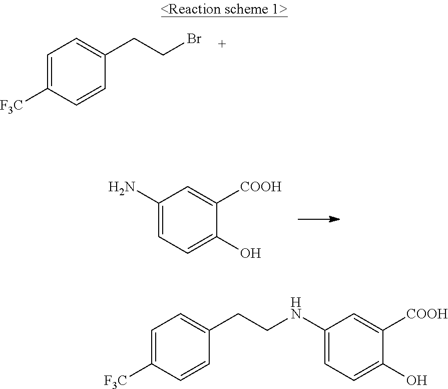

Reaction condition: triethylamine, tetrabutylammonium, N,N-dimethylformamide, room temperature, 3 hours.

5-aminosalicylic acid is added into N,N-dimethylformamide. Diethylamine, organic base, and 1-(2-bromoethyl)-4-trifluoromethylbenzene are added, and then mixed at room temperature for 3 hours to get the compound of the present invention.

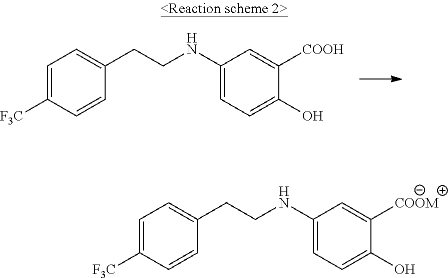

In the scheme 2, M is a pharmaceutically acceptable metal or basic organic compound such as diethylamine, lithium, sodium and potassium.

The pharmaceutically acceptable salt of the compound according to the present invention can be easily prepared. For example, diethylamine salt can be prepared as follows. Firstly, the compound is dissolved into alcohol, and then diethylamine is drop-wisely added into the solution. The solution is mixed and vacuum-evaporated to get a residue. Ether is added to the residue to crystallize the salt. Alkali metal salt can be prepared as follows. Desirable salt is prepared with inorganic reagent such as lithium hydroxide, lithium acetate, sodium hydroxide, sodium 2-ethylhexanoate, sodium acetate, potassium acetate and potassium hydroxide under solvent like alcohol, acetone, acetonitrile, etc. Then, the salt is obtained from freeze-drying.

Another aspect of the present invention is based on the discovery disclosed herein that a class of 2-hydroxy-alkylamino-benzoic acid derivatives or their pharmaceutically acceptable salts inhibits production or aggregation of beta-amyloid (Aβ).

Therefore, the present invention in one embodiment provides a method of inhibiting Aβ production or reducing the undesirable levels of Aβ for treating Alzheimer's disease (AD) by administering to the mammal in need thereof a therapeutically effective amount of a 2-hydroxy-alkylamino-benzoic acid derivative represented by the following formula or a pharmaceutically acceptable salt thereof:

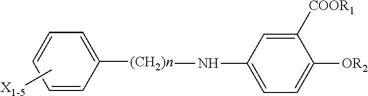

wherein,

n is an integer of 2 or 3.

R₁ is hydrogen or alkyl;

R₂ is hydrogen, alkyl or alkanoyl; and

X is independently halogen, haloalkyl or haloalkoxy.

In the above formula, preferably, alkyl is C₁-C₄ alkyl, and more preferably C₁-C₂ alkyl. Alkyl described above includes, but is not limited to, methyl, ethyl, propyl, isopropyl, n-butyl, sec-butyl, and tert-butyl. Preferably, Alkoxy is C₁-C₄alkoxy, and more preferably C₁-C₂ alkoxy. Alkoxy described above includes, but is not limited to, methoxy, ethoxy, and propanoxy. Halogen includes, but is not limited to, fluoride, chloride, bromide, and iodide. Alkanoyl is C₂-C₁₀ alkanoyl, and more preferably C₃-C₅ alkanoyl. Alkanoyl described above includes, but is not limited to, ethanoyl, propanoyl, and cyclohexanecarbonyl.

Considering the efficacy of inhibiting Aβ production, preferable examples of the 2-hydroxy-alkylamino-benzoic acid derivative include, but are not limited to,
2-Hydroxy-5-(2-(4-trifluoromethyl-phenyl)-ethylamino)-benzoic acid (hereinafter, referred to as chemical_{—}01),
5-(2-(2-Chloro-phenyl)-ethylamino)-2-hydroxy-benzoic acid (hereinafter, referred to as chemical_{—}02),
2-Hydroxy-5-(2-(4-trifluoromethoxy-phenyl)-ethylamino)-benzoic acid (hereinafter, referred to as chemical_{—}03),
5-(2-(3,4-Difluoro-phenyl)-ethylamino)-2-hydroxy-benzoic acid (hereinafter, referred to as chemical_{—}04),
5-(2-(2,4-Dichloro-phenyl)-ethylamino)-2-hydroxy-benzoic acid (hereinafter, referred to as chemical_{—}05),
5-(2-(3,5-Bis-trifluoromethyl-phenyl)-ethylamino)-2-hydroxy-benzoic acid (hereinafter, referred to as chemical_{—}06),
2-Hydroxy-5-(3-(4-trifluoromethyl-phenyl)-propylamino)-benzoic acid (hereinafter, referred to as chemical_{—}07),
5-(3-(3,4-Dichloro-phenyl)-propylamino)-2-hydroxy-benzoic acid (hereinafter, referred to as chemical_{—}08).

Based on their therapeutic efficacy, chemical_{—}01, chemical_{—}02 and chemical_{—}05 are more preferable, and chemical_{—}01 and chemical_{—}02 are most preferable.

The 2-hydroxy-alkylamino-benzoic acid derivatives of the present invention can be administered as a form of pharmaceutically acceptable salt. The pharmaceutically acceptable acid addition salts of the present compounds can be formed of the compound itself, or of any of its esters, and include the pharmaceutically acceptable salts which are often used in pharmaceutical chemistry. For example, salts may be formed with organic or inorganic acids. Suitable organic acids include maleic, fumaric, benzoic, ascorbic, succinic, methanesulfonic, benzenesulfonic, toluenesulfonic, acetic, oxalic, trifluoroacetic, propionic, tartaric, salicylic, citric, gluconic, lactic, mandelic, cinnamic, aspartic, stearic, palmitic, formic, glycolic, glutamic, and benzenesulfonic acids. Suitable inorganic acids include hydrochloric, hydrobromic, sulfuric, phosphoric, and nitric acids. Additional salts include chloride, bromide, iodide, bisulfate, acid phosphate, isonicotinate, lactate, acid citrate, oleate, tannate, pantothenate, bitartrate, gentisinate, gluconate, glucaronate, saccharate, ethanesulfonate, p-toluenesulfonate, and pamoate (i.e., 1,1′-methylene-bis-(2-hydroxy-3-naphthoate)) salts. The term “pharmaceutically acceptable salt” is intended to encompass any and all acceptable salt forms.

Pharmaceutically acceptable salts can be formed by conventional and known techniques, such as by reacting a compound of this invention with a suitable acid as disclosed above. Such salts are typically formed in high yields at moderate temperatures, and often are prepared by merely isolating the compound from a suitable acidic wash in the final step of the synthesis. The salt-forming acid may dissolved in an appropriate organic solvent, or aqueous organic solvent, such as an alkanol, ketone or ester. On the other hand, if the compound of the present invention is desired in the free base form, it may be isolated from a basic final wash step, according to known techniques. For example, a typical technique for preparing hydrochloride salt is to dissolve the free base in a suitable solvent, and dry the solution thoroughly, as over molecular sieves, before bubbling hydrogen chloride gas through it.

In addition, some of 2-hydroxy-alkylamino-benzoic acid derivatives of the present invention may be in a hydrated form, and may exist as a solvated or unsolvated form. A part of compounds exist as crystal form or amorphous form, and any physical form is included in the scope of the present invention.

The 2-hydroxy-alkylamino-benzoic acid derivatives of the present invention may contain one or more asymmetric carbon atoms and therefore exist in two or more stereoisomeric forms. The present invention includes a method administering these individual stereoisomers of the compounds of the present invention.

The compounds of the present invention can be administered as a form of tablets, capsules, powders, mixtures, solutions, suspensions or other suitable pharmaceutical formulation forms. These pharmaceutical formulations may comprise at least one pharmaceutically acceptable excipient for easiness of manufacturing, and appearance and stability of the formulation.

Routes of administration of the compounds of the present invention include, but are not limited to, oral, topical, subcutaneous, transdermal, subdermal, intra-muscular, intra-peritoneal, intravesical, intra-articular, intra-arterial, intra-venous, intra-dermal, intra-cranial, intra-lesional, intra-tumoral, intra-ocular, intra-pulmonary, intra-spinal, intraprostatic, placement within cavities of the body, nasal inhalation, pulmonary inhalation, impression into skin and electrocorporation.

To produce pharmaceutical formulations for orally administering the compounds according to the present invention, the selected compound may be mixed with a solid excipients, for example, a diluent such as lactose, mannitol, microcrystalline cellulose and corn starch; a binder such as gelatin and polyvinylpyrrolidone; a disintegrator such as sodium starch glycolate and cross-carmellose sodium; a lubricant such as magnesium stearate, wax and so on; and the like, and then compressed into tablets. If coated tablets are required, the tablet cores prepared above may be coated with a coating material such as gelatin, hydroxypropylmethylcellulose and so on.

For the formulation of soft gelatin capsules, the two active substances may be admixed with, for example, a vegetable oil or poly-ethylene glycol. Hard gelatin capsules may contain granules of the two active substances using a method well known to those skilled in the art.

Liquid formulation for oral application may be in the form of syrups, solutions or suspensions, and such liquid formulations may contain coloring agents, flavoring agents, sugar, stabilizers, surfactants, thickening agent or other excipients known to those skilled in the art.

Solutions for parenteral applications by injection can be prepared in an aqueous solution of a water-soluble pharmaceutically acceptable salt of the two active ingredients, preferably in a concentration of from about 0.1% to about 20% by weight. These solutions may also contain stabilizing agents, buffering agents and/or pH-adjusting agents, and may be conveniently prepared by conventional methods.

The particular therapeutic agent administered, the amount per dose, the dose schedule and the route of administration should be decided by the practitioner using methods known to those skilled in the art and will depend on the type of neurological disease, the severity of the disease, the location of the disease and other clinical factors such as the size, weight and physical condition of the recipient. In addition, in vitro assays may optionally be employed to help identify optimal ranges for sequence administration.

For the purpose of this invention, daily dosage of the 2-hydroxy-alkylamino-benzoic acid derivatives or their pharmaceutically acceptable salts may be in the range of about 0.1 mg-100 g/kg bodyweight, preferably about 0.5 mg-10 g/kg bodyweight, more preferably about 1 mg-1 g/kg bodyweight. As occasion demands, the compounds of the present invention can be administered in small doses 1 to 4 times a day over variable times from weeks to months.

Hereinafter, embodiments of the present invention are described in considerable detail to help those skilled in the art further understand the present disclosure. However, the following examples are offered by way of illustration and are not intended to limit the scope of the invention. It is apparent that various changes may be made without departing from the spirit and scope of the invention or sacrificing all of its material advantages.

### Synthesis Example 1

### Preparation of 2-hydroxy-5-(2-(4-trifluoromethyl-phenyl)-ethylamino)-benzoic acid (chemical_{—}01)

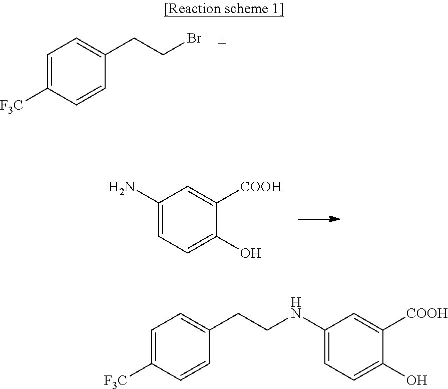

Reagent: triethylamine, tetrabutylammonium, N,N-dimethylformamide, room temperature, 3 hours

5-aminosalicylic acid is added into N,N-dimethylformamide. Diethylamine, organic base, and 1-(2-bromoethyl)-4-trifluoromethylbenzene are added, and then mixed at room temperature for 3 hours. The reaction mixture was filtered, and the resulting solid was washed with water three times and diethyl ether, then dried, to give the compound as pale yellow solid.

### Synthesis Example 2

### Preparation of 5-(2-(2-chloro-phenyl)-ethylamino)-2-hydroxy-benzoic acid (chemical_{—}02)

According to the similar procedure as that of Synthesis Example 1, 5-(2-(2-Chloro-phenyl)-ethylamino)-2-hydroxy-benzoic acid was obtained.

¹H-NMR: 7.35 (q, 2H), 7.22 (t, 2H), 7.09 (s, 1H), 6.82 (d, 1H), 6.57 (d, 1H), 3.24 (t, 2H), 2.84 (t, 2H)

### Synthesis Example 3

### Preparation of 2-hydroxy-5-(2-(4-trifluoromethoxy-phenyl)-ethylamino)-benzoic acid (chemical_{—}03)

According to the similar procedure as that of Synthesis Example 1, 2-Hydroxy-5-(2-(4-trifluoromethoxy-phenyl)-ethylamino)-benzoic acid was obtained.

¹H-NMR: 7.35 (d, 2H), 7.21 (d, 2H), 6.98 (d, 1H), 6.84 (q, 1H), 6.74 (d, 1H), 3.20 (t, 2H), 2.84 (t, 2H)

### Synthesis Example 4

### Preparation of 5-(2-(3,4-difluoro-phenyl)-ethylamino)-2-hydroxy-benzoic acid (chemical_{—}04)

According to the similar procedure as that of Synthesis Example 1, 5-(2-(3,4-Difluoro-phenyl)-ethylamino)-2-hydroxy-benzoic acid was obtained.

¹H-NMR: 7.31 (m, 2H), 7.11 (s, 1H), 6.94 (d, 1H), 6.87 (d, 1H), 6.74 (d, 1H), 3.17 (t, 2H), 2.87 (t, 2H)

### Synthesis Example 5

### Preparation of 5-(2-(2,4-dichloro-phenyl)-ethylamino)-2-hydroxy-benzoic acid (chemical_{—}05)

According to the similar procedure in Synthesis Example 1, 5-(2-(2,4-Dichloro-phenyl)-ethylamino)-2-hydroxy-benzoic acid was obtained.

¹H-NMR: 7.49 (s, 1H), 7.35-7.28 (m, 2H), 7.03 (d, 1H), 6.88 (t, 1H), 6.74 (d, 1H), 3.19 (t, 2H), 2.91 (t, 2H)

### Synthesis Example 6

### Preparation of 5-(2-(3,5-bis-trifluoromethyl-phenyl)-ethylamino)-2-hydroxy-benzoic acid (chemical_{—}06)

According to the similar procedure in Synthesis Example 1, 5-(2-(3,5-Bis-trifluoromethyl-phenyl)-ethylamino)-2-hydroxy-benzoic acid was obtained.

¹H-NMR: 7.97 (s, 2H), 7.88 (s, 1H), 7.28 (s, 1H), 7.15 (t, 1H), 6.82 (d, 1H), 3.42 (t, 2H), 3.09 (t, 2H)

### Synthesis Example 7

### Preparation of 2-hydroxy-5-(3-(4-trifluoromethyl-phenyl)-propylamino)-benzoic acid (chemical_{—}07)

According to the similar procedure in Synthesis Example 1, 2-Hydroxy-5-(3-(4-trifluoromethyl-phenyl)-propylamino)-benzoic acid was obtained.

¹H-NMR: 7.61 (d, 2H), 7.43 (d, 2H), 6.97 (s, 1H), 6.85 (t, 1H), 6.78 (d, 1H), 3.04 (t, 2H), 2.78 (t, 2H), 1.87 (m, 2H)

### Synthesis Example 8

### Preparation of 5-(3-(3,4-dichloro-phenyl)-propylamino)-2-hydroxy-benzoic acid (chemical_{—}08)

According to the similar procedure in Synthesis Example 1, 5-(3-(3,4-Dichloro-phenyl)-propylamino)-2-hydroxy-benzoic acid was obtained.

¹H-NMR: 7.45 (q, 2H), 7.15 (d, 1H), 7.05 (d, 1H), 6.92 (q, 1H), 6.77 (d, 1H), 2.96 (t, 2H), 2.66 (t, 2H), 1.82 (m, 2H)

### Example 1

### Mixed Cortical Cell Cultures of Neurons and Glia

For mixed neuron-glia culture, mouse cerebral cortices were removed from brains of the 11-15 day-old-fetal mice (E11-15), gently triturated and plated on 24 well plates (2×10⁵ cells/plate) precoated with 100 μg/ml poly-D-lysine and 4 pg/ml laminin. Cultures were maintained at 37° C. in a humidified 5% CO₂ atmosphere. Plating media consist of Eagles minimal essential media (MEM, Earles salts, supplied glutamine-free) supplemented with 5% horse serum, 5% fetal bovine serum, 26.5 mM bicarbonate, 2 mM glutamine, and 21 mM glucose.

After 7-8 days in vitro (DIV 7-8), 10 μM cytosine arabinofuranoside (Ara-C) was included to halt overgrowth of glia. The drug treatment was carried on DIV 11-15 cortical cell culture. Overall neuronal cell injury was assessed by measuring amount of lactate dehydrogenase (LDH) released into the bathing medium 24 hr after neurotoxic insults as previously described (Koh and Choi, J Neurosci Methods 20:83-90, 1987).

### Example 2

### Blockade of Free Radical Neurotoxicity by Vitamin E, Trolox, 2-hydroxy-TTBA, 2-hydroxy-TPEA, BAS, NBAS, CBAS, MBAS, FBAS, PBAS, NPAA, NPPAA and TBAS

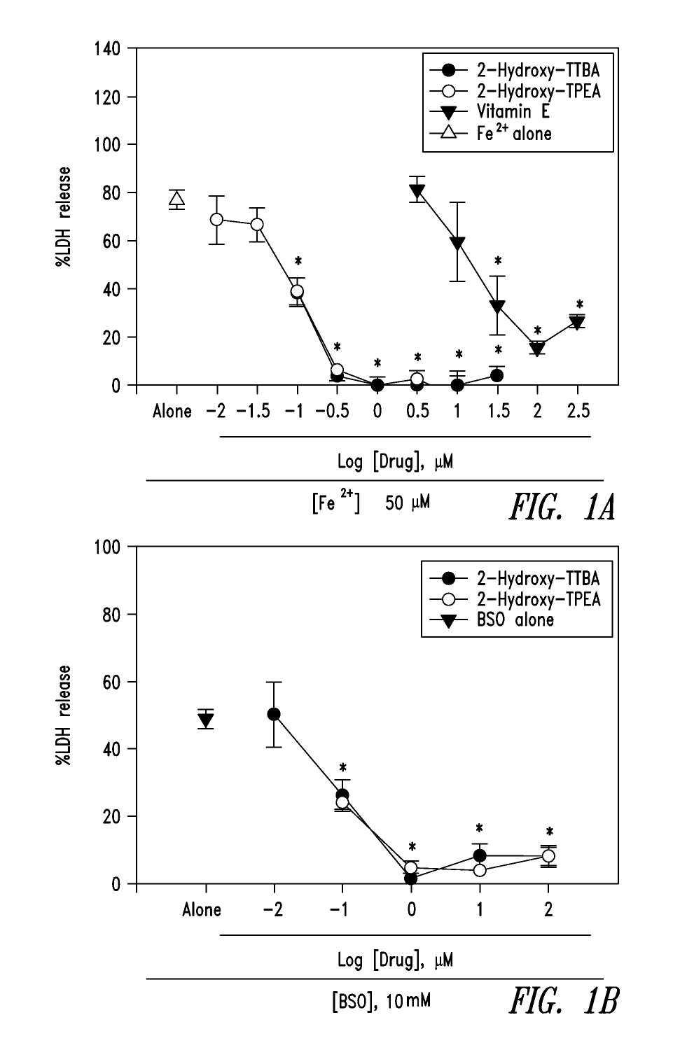

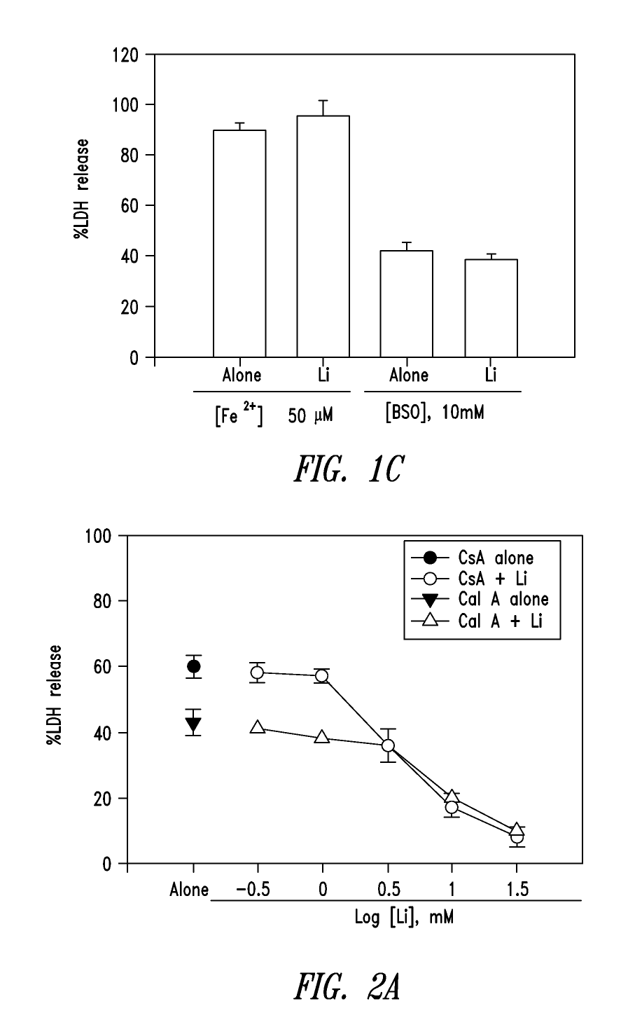

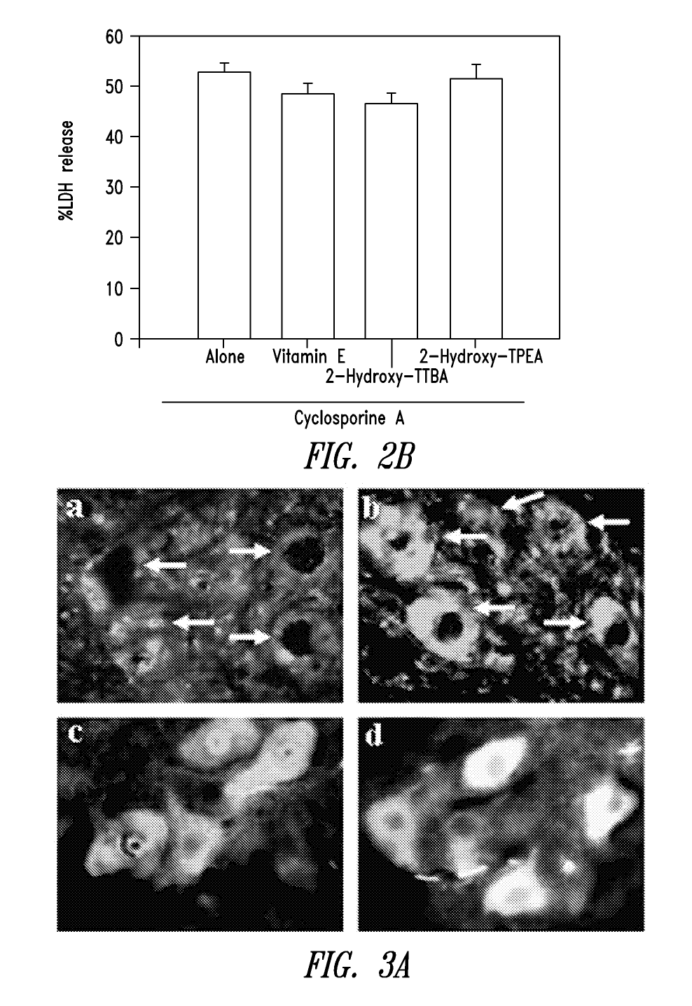

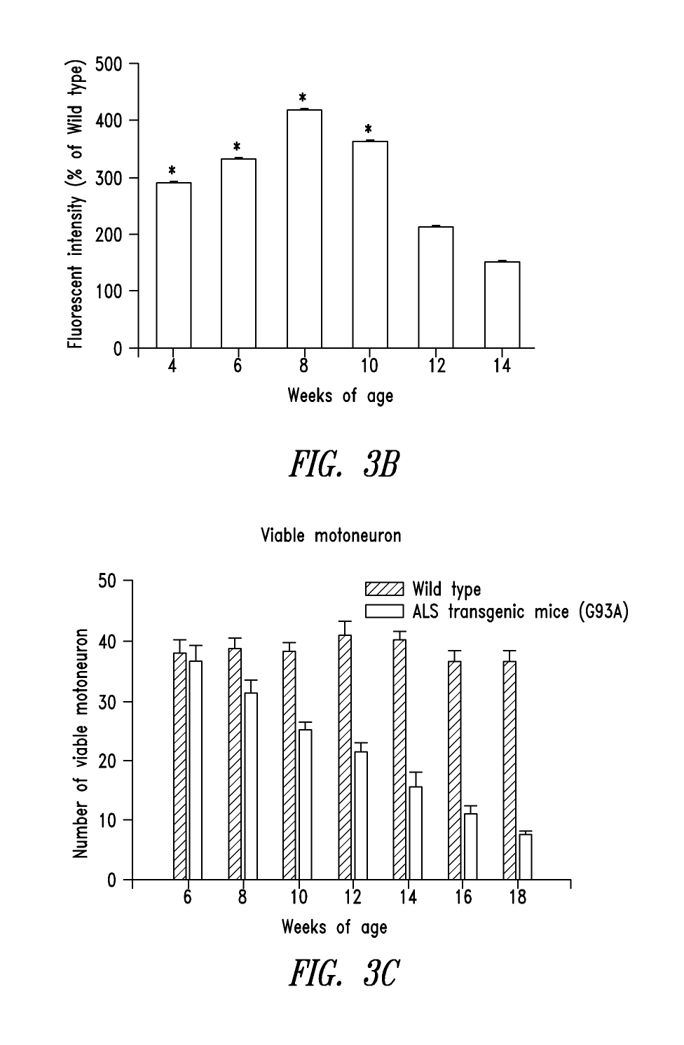

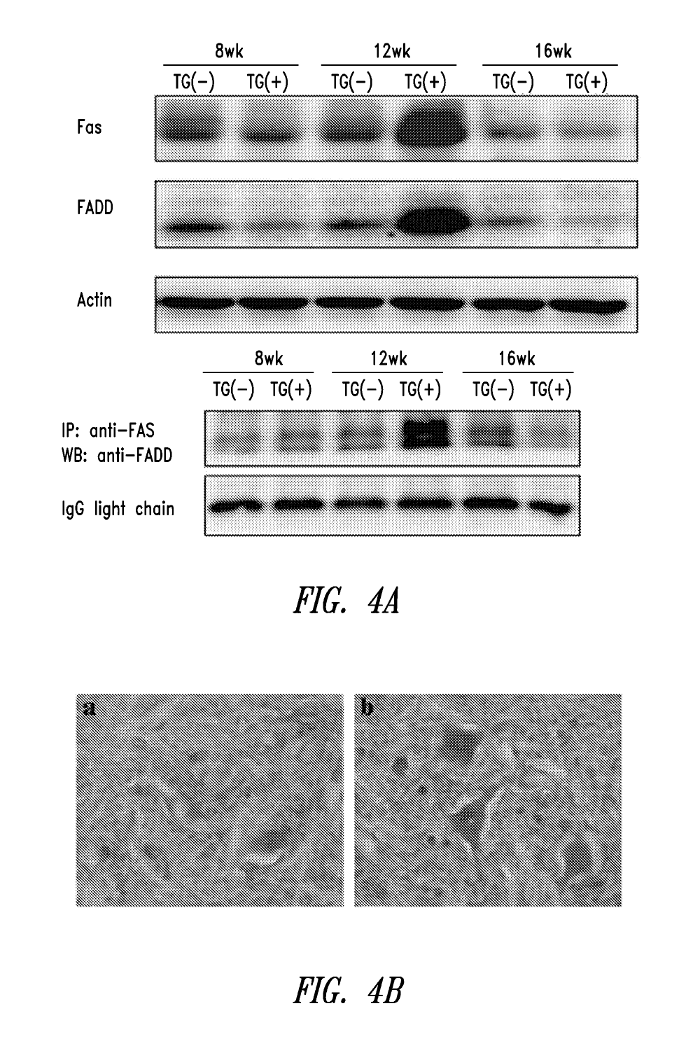

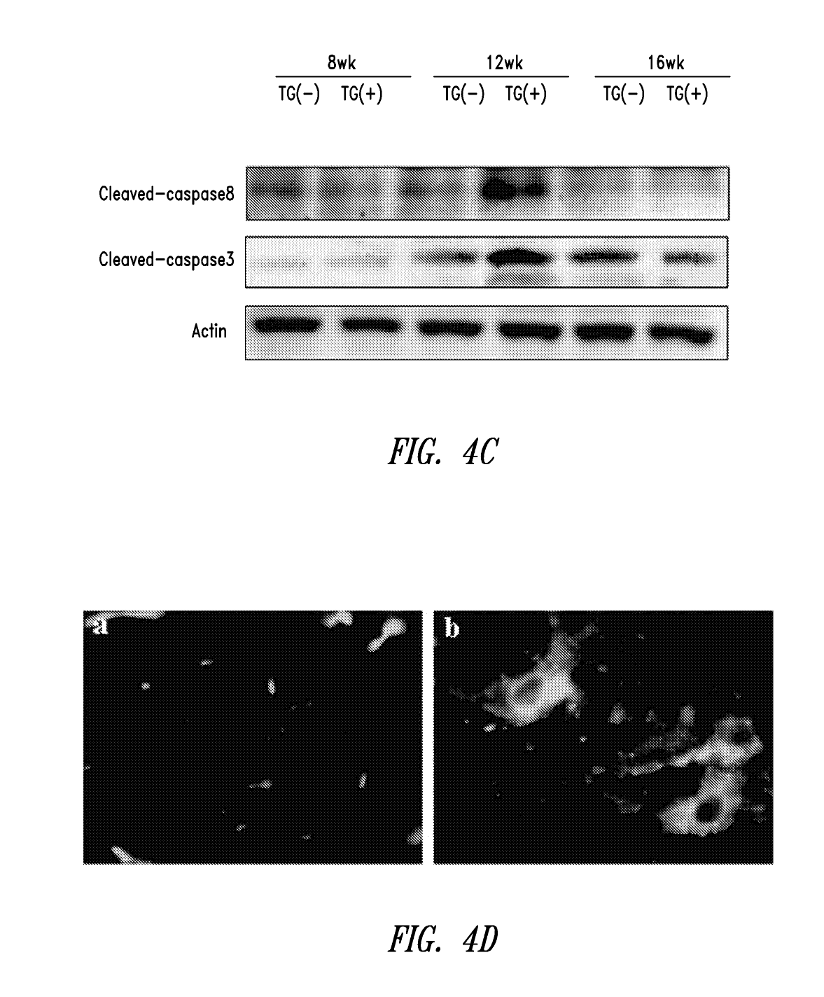

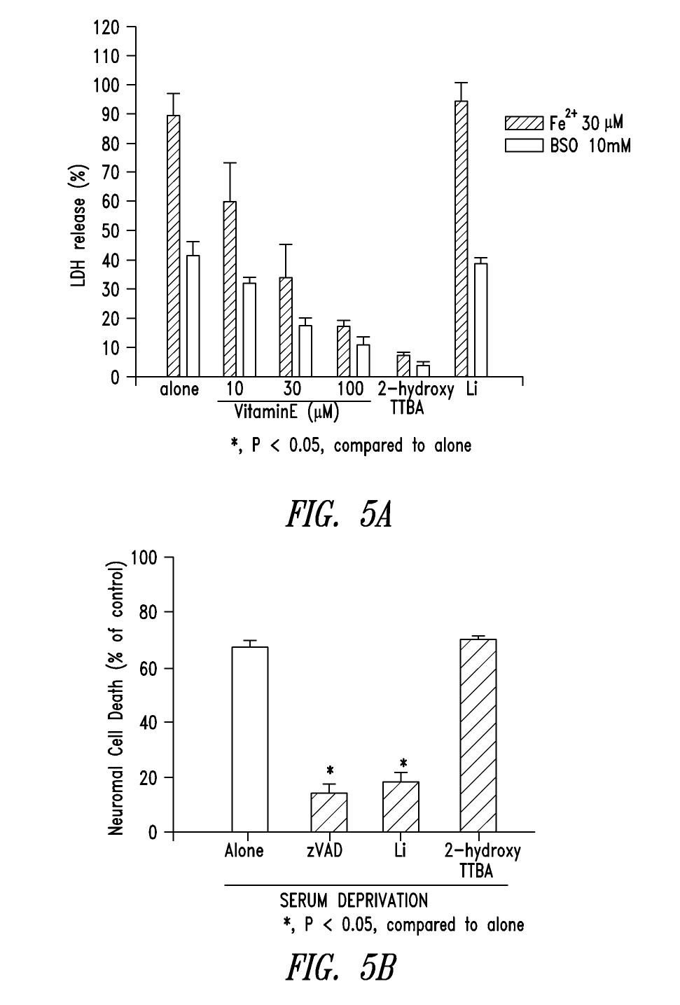

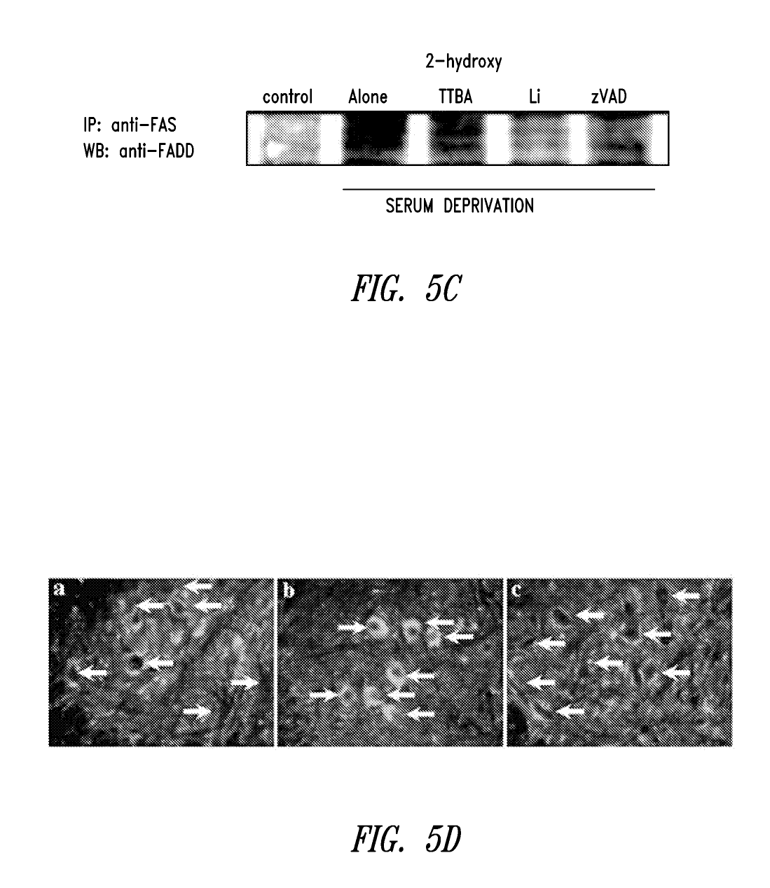

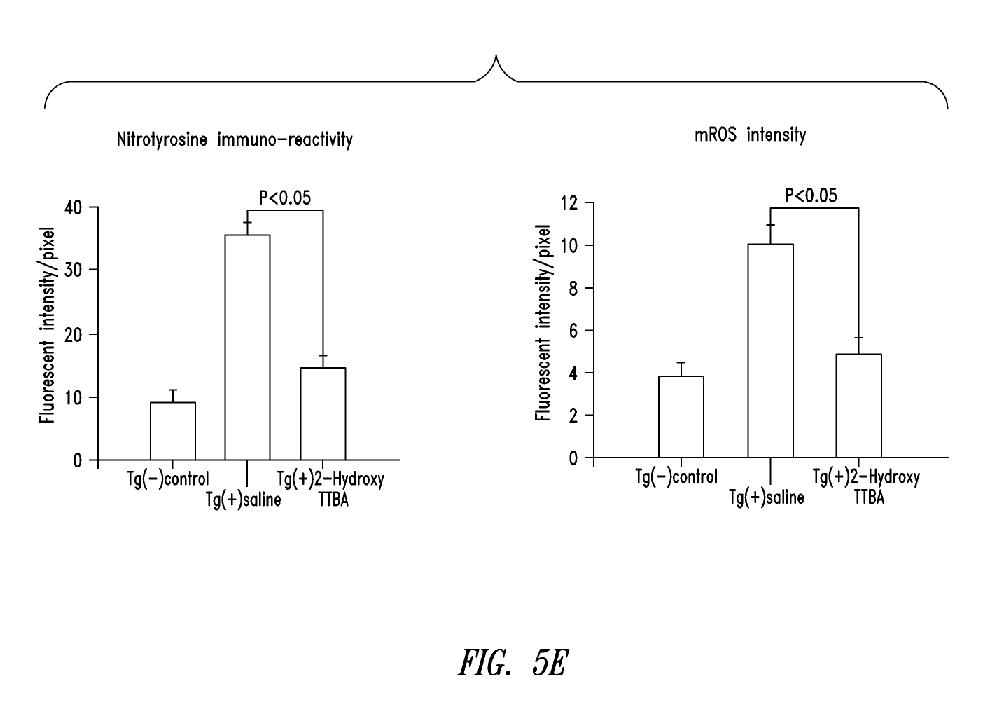

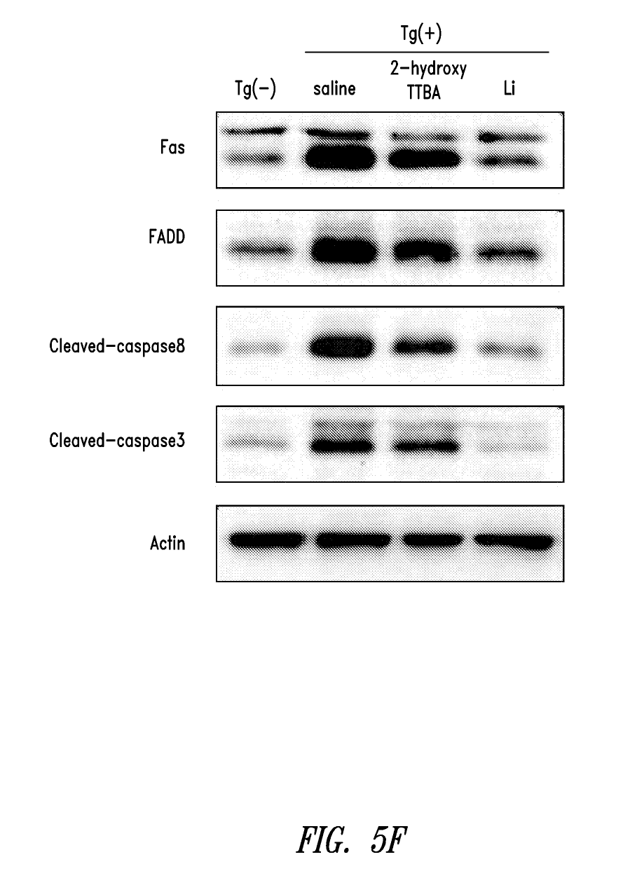

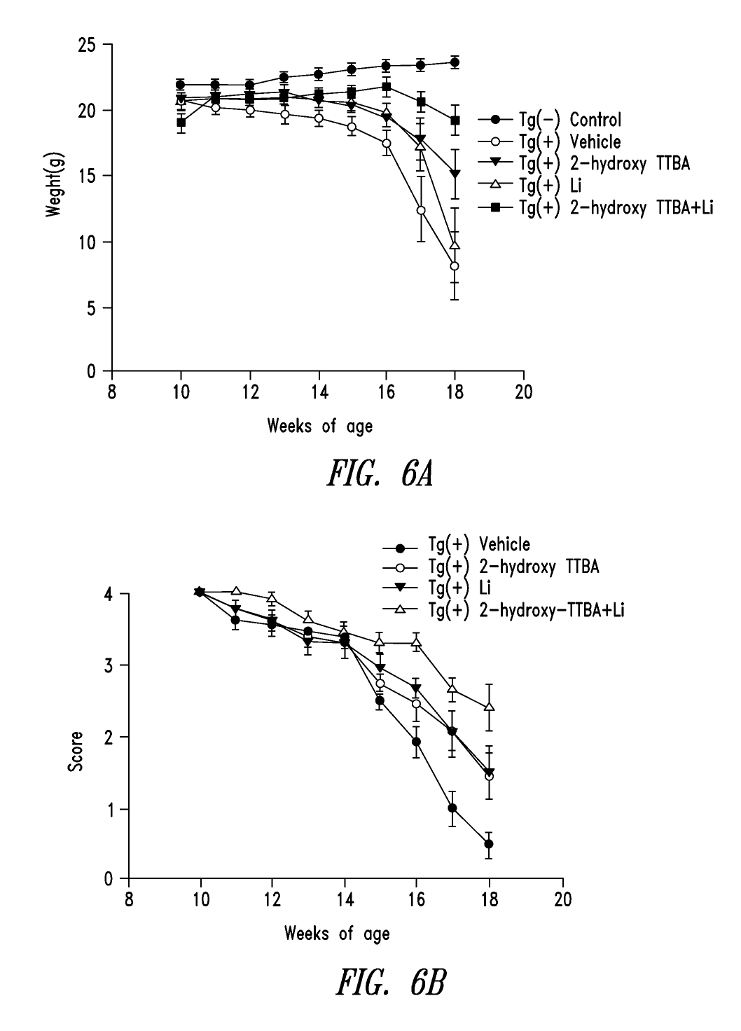

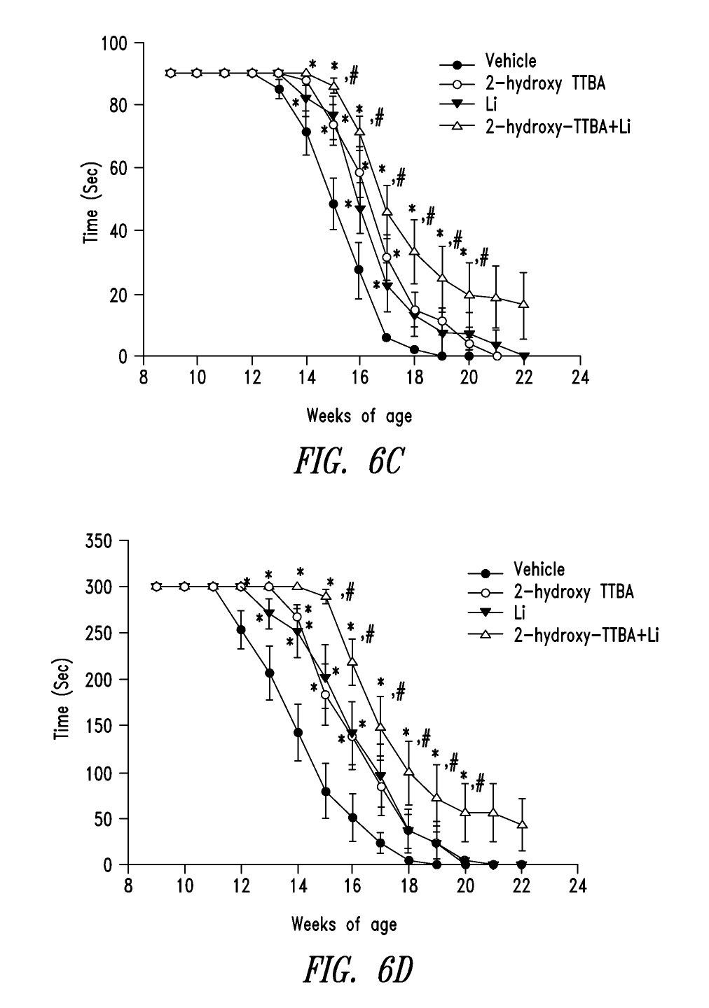

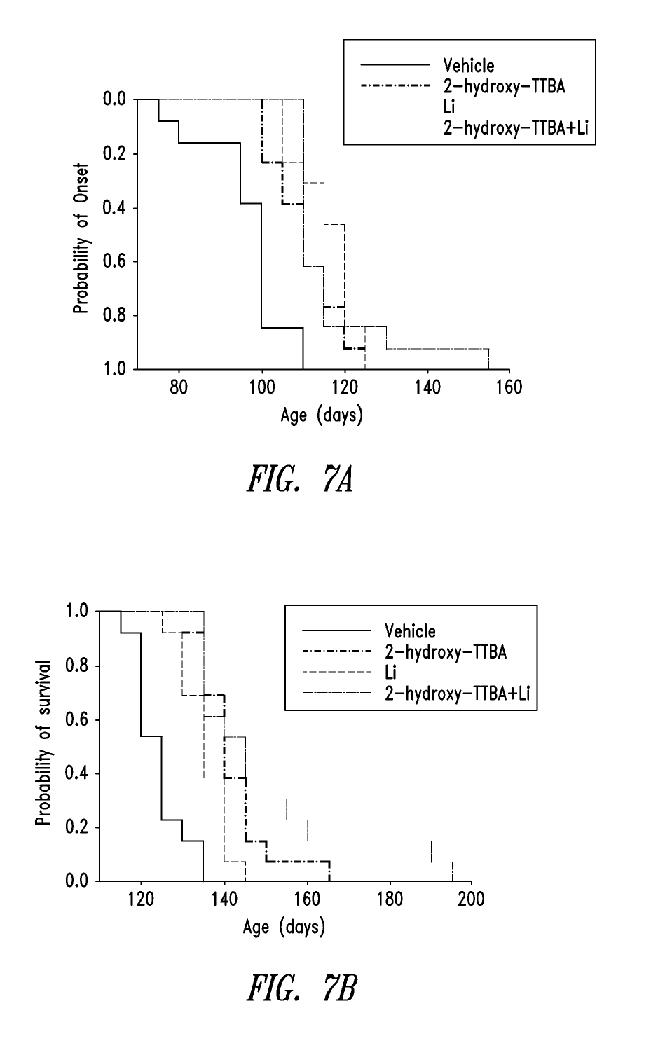

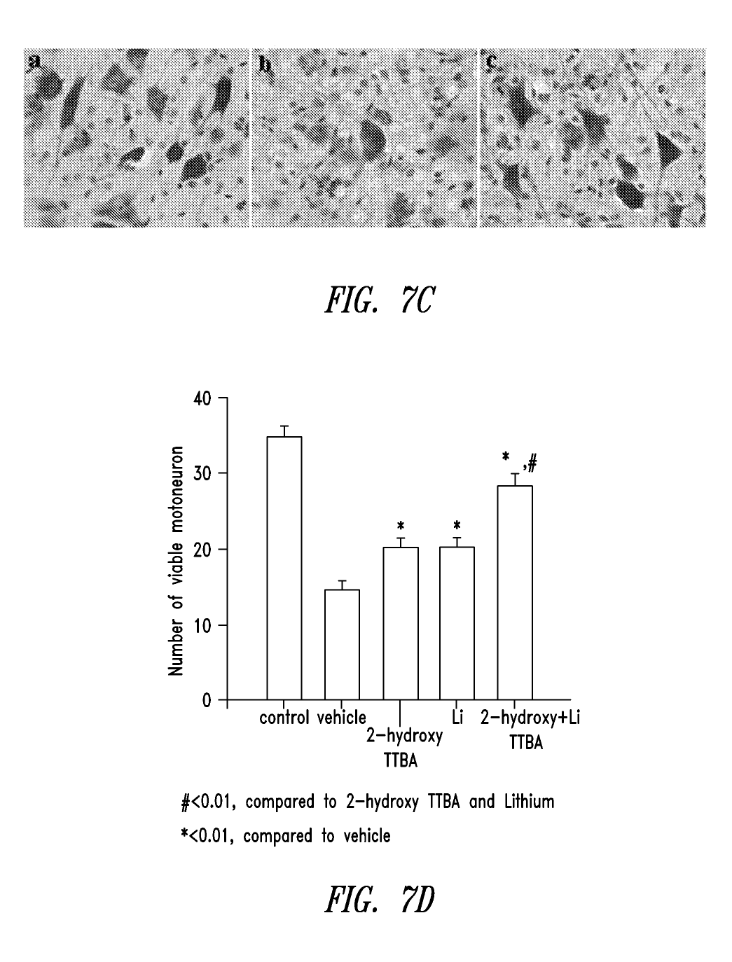

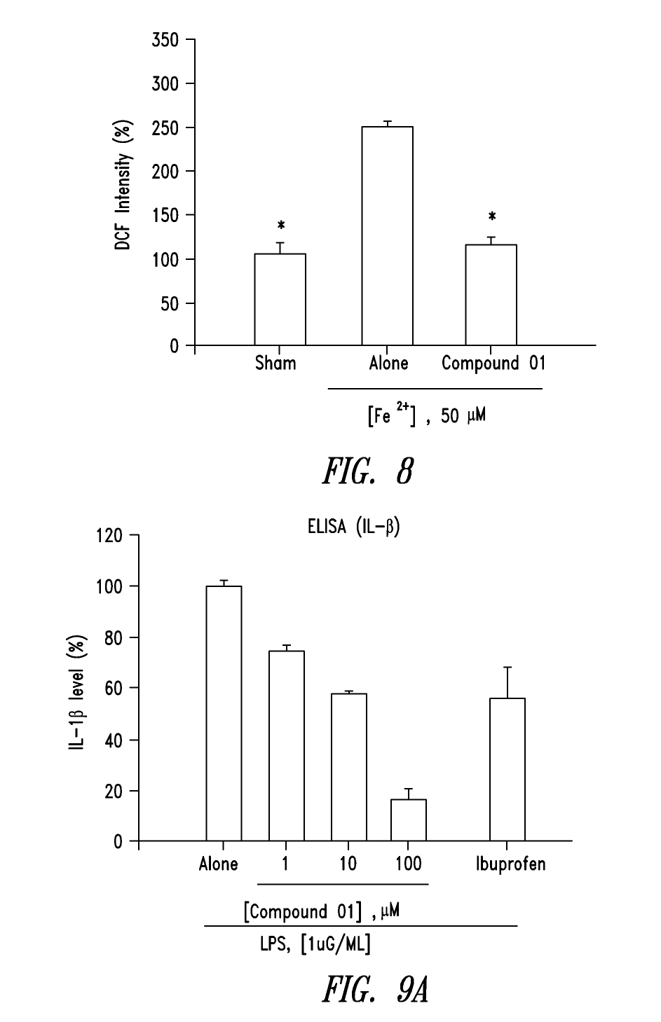

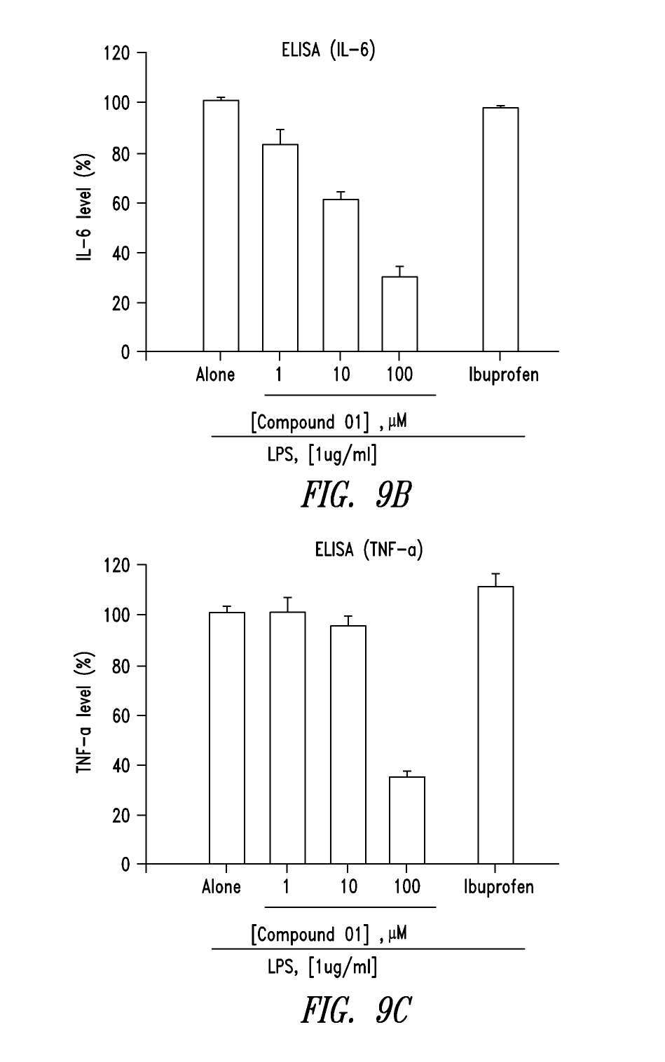

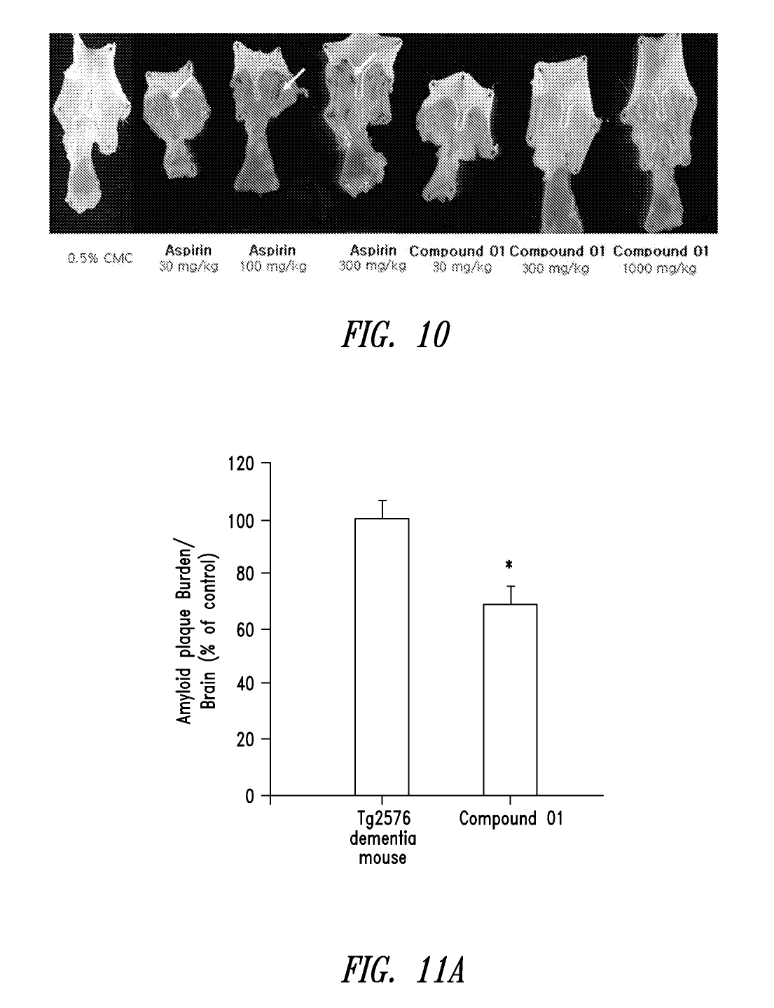

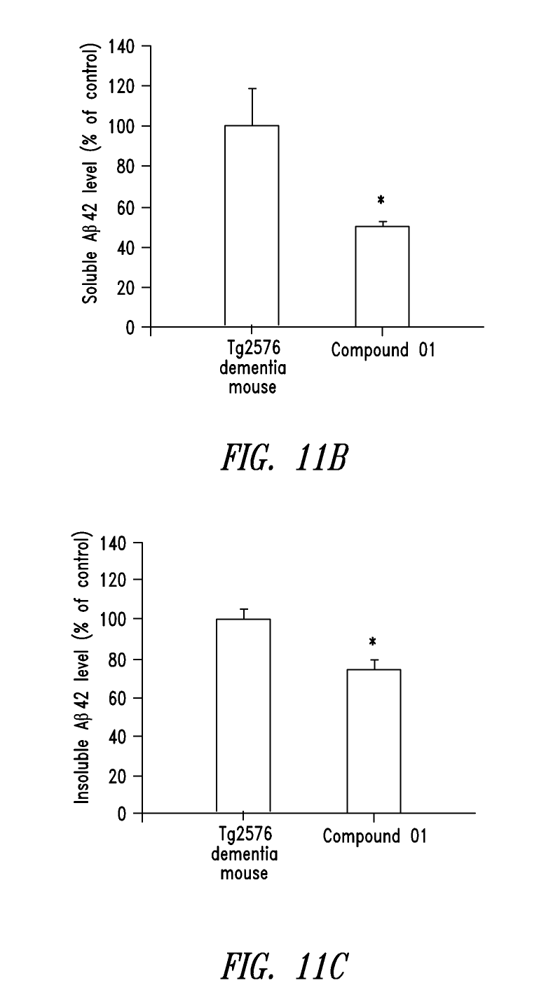

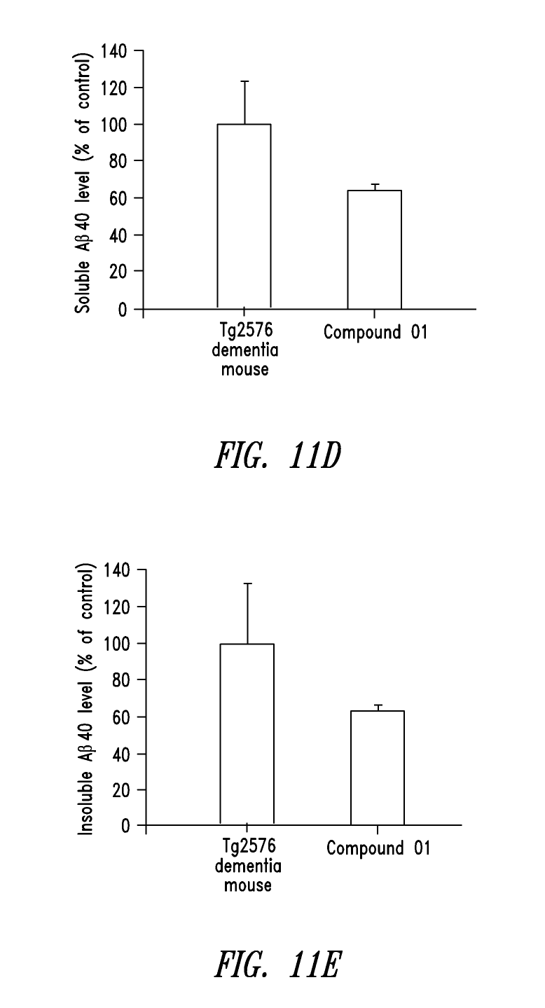

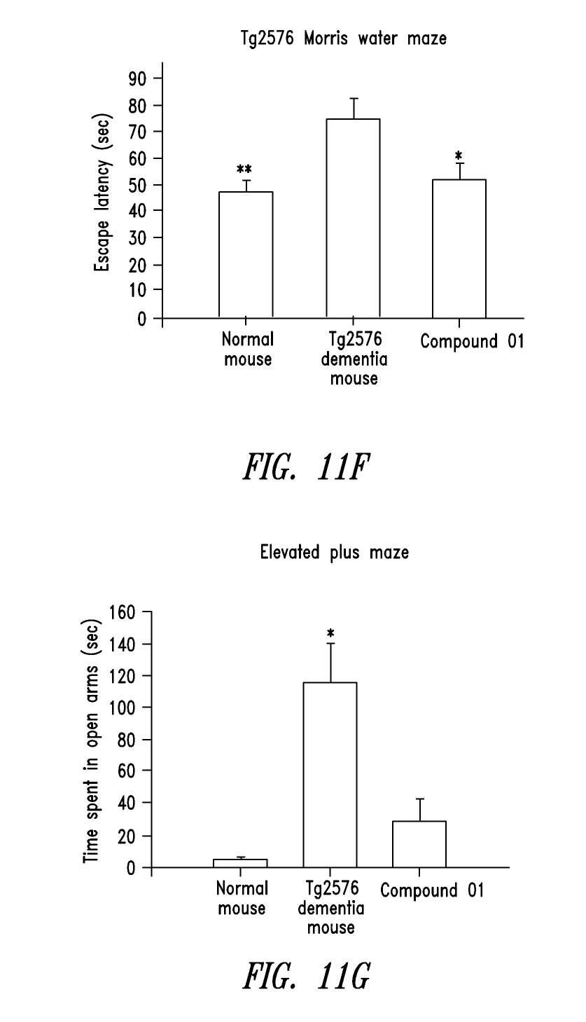

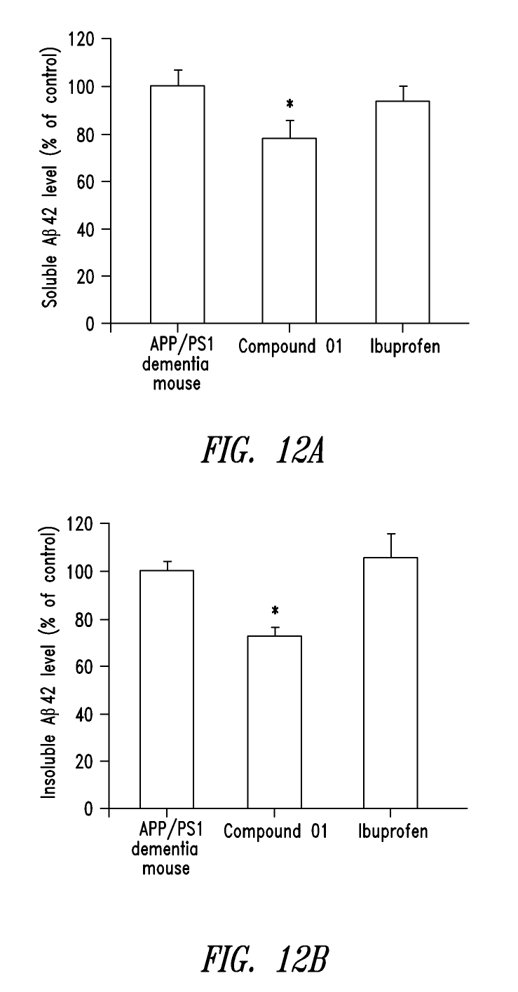

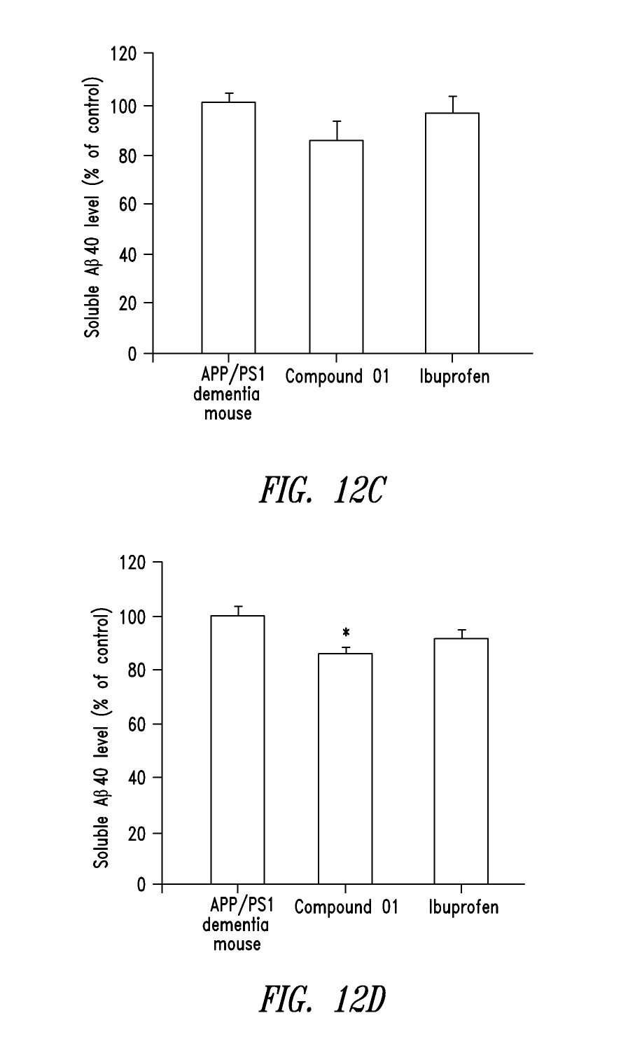

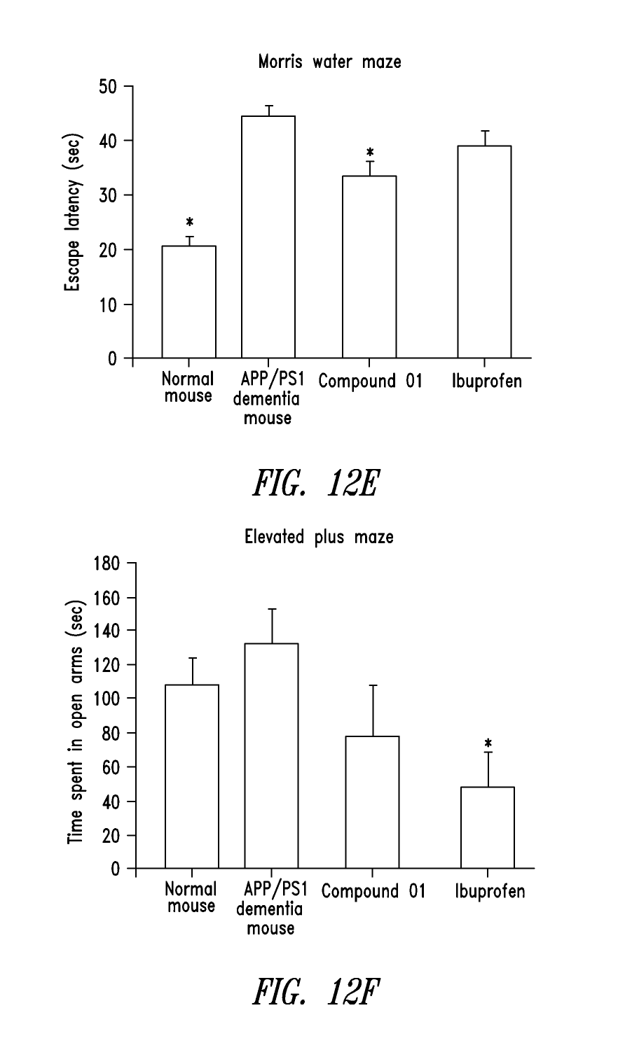

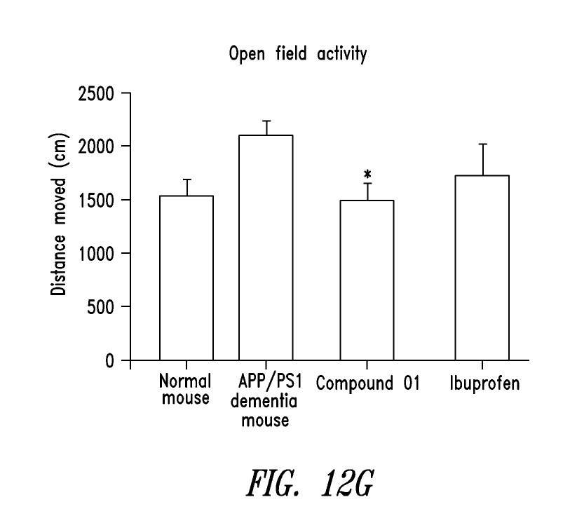

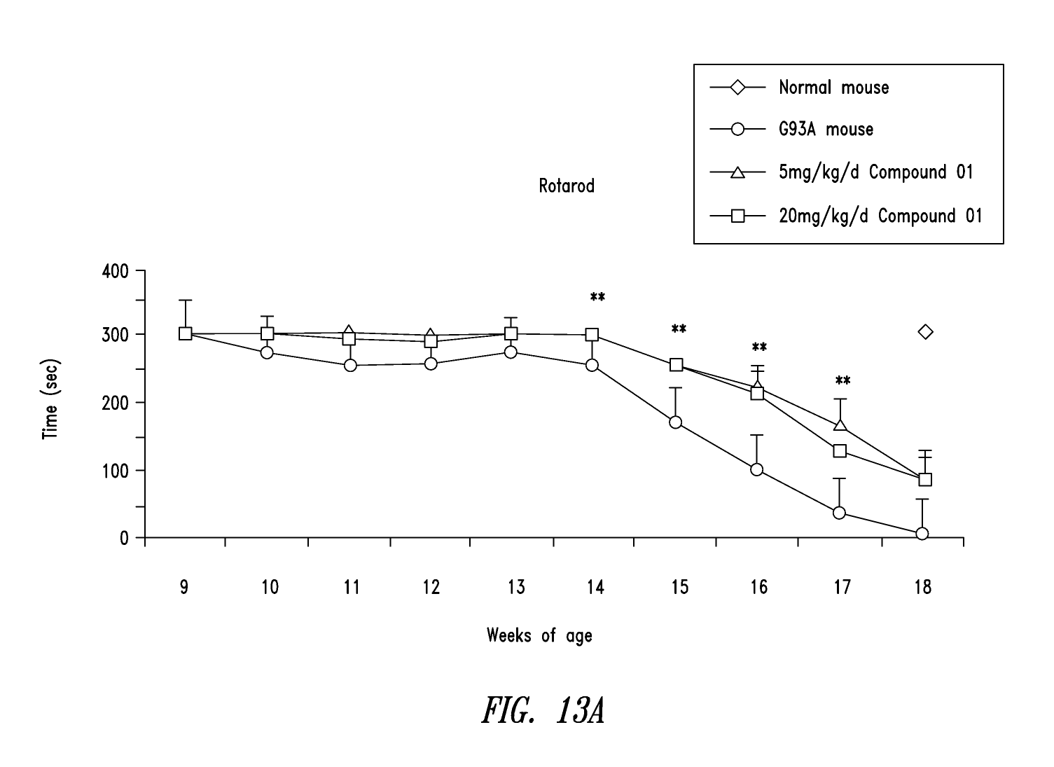

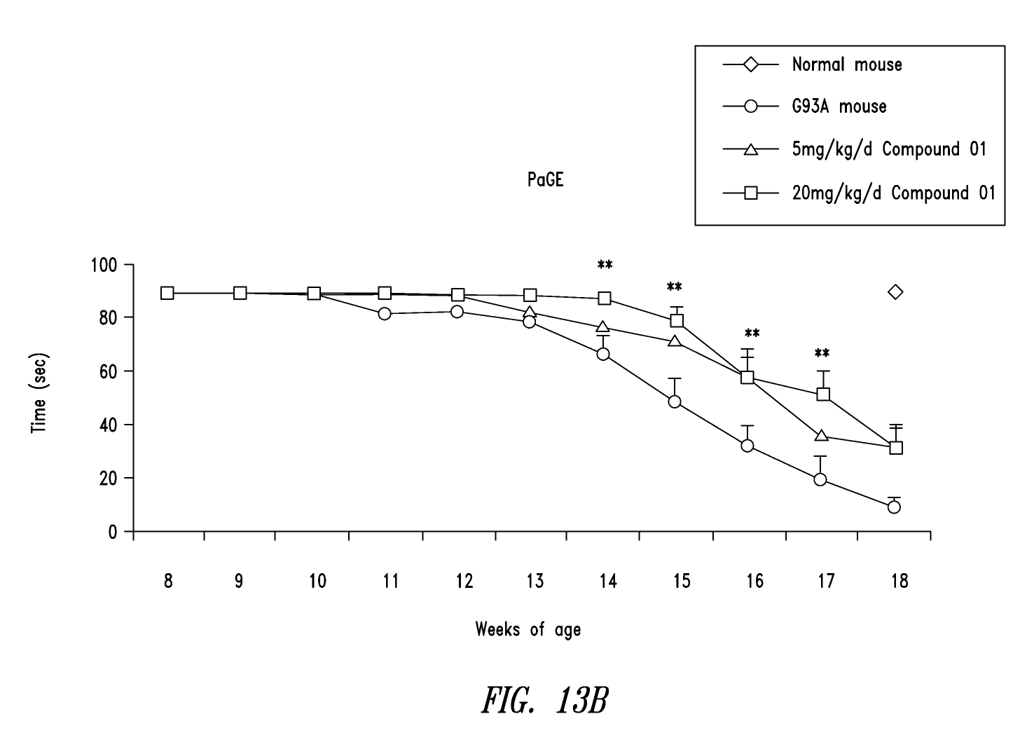

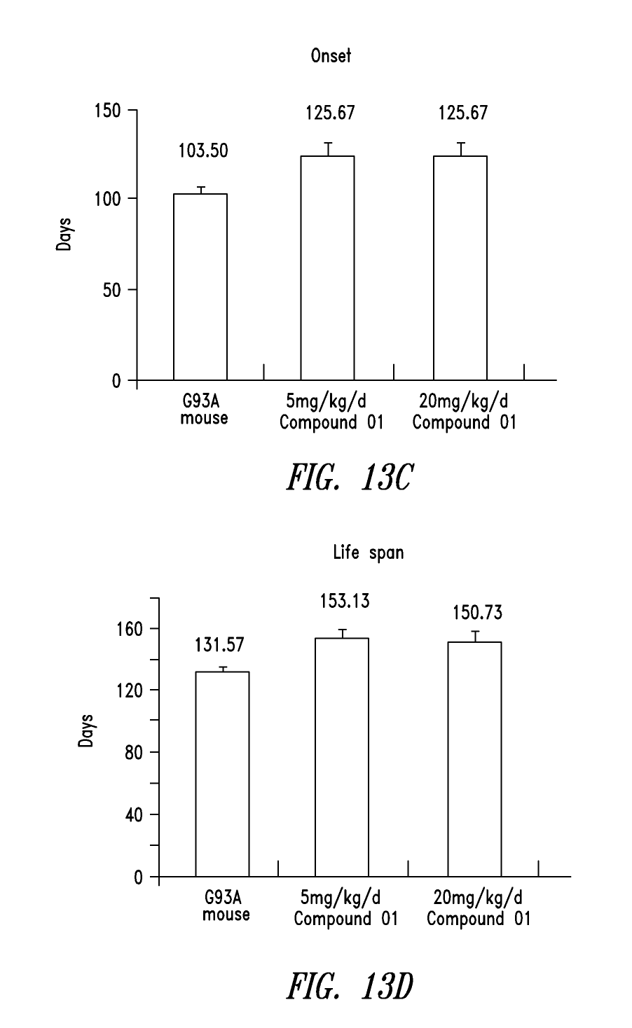

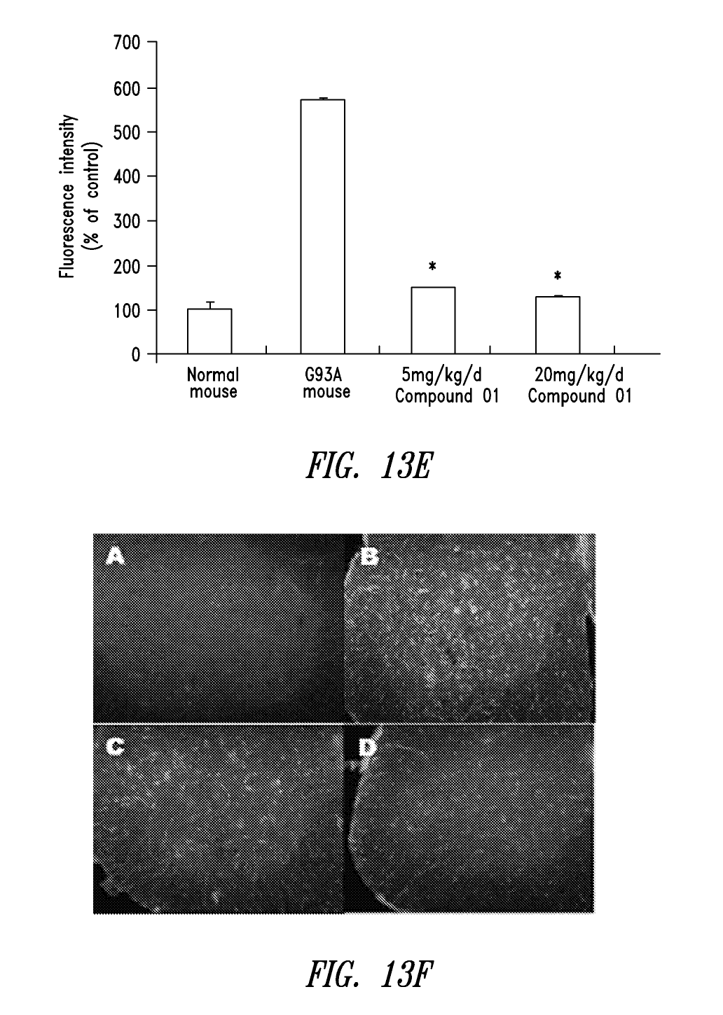

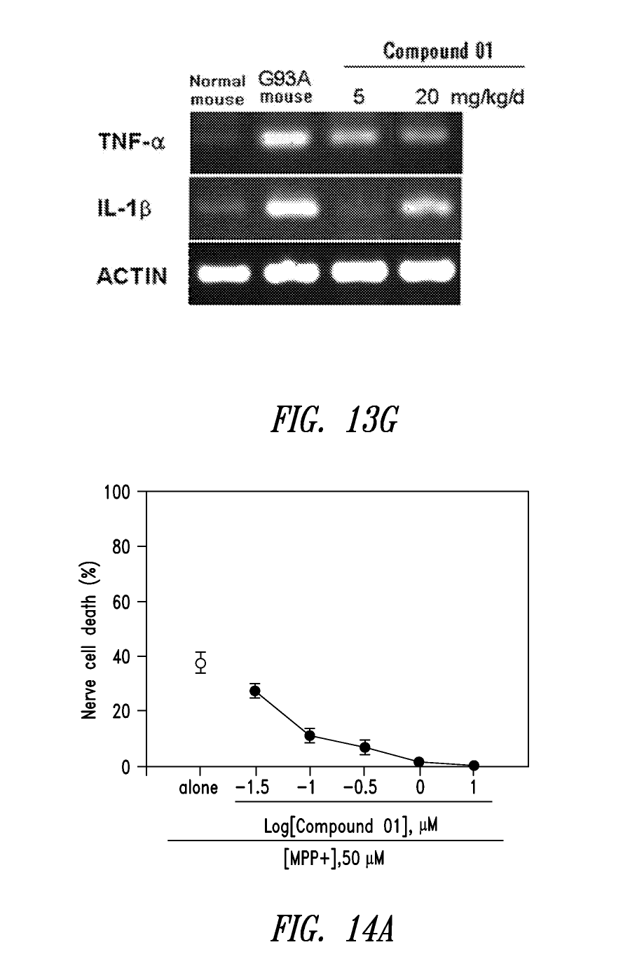

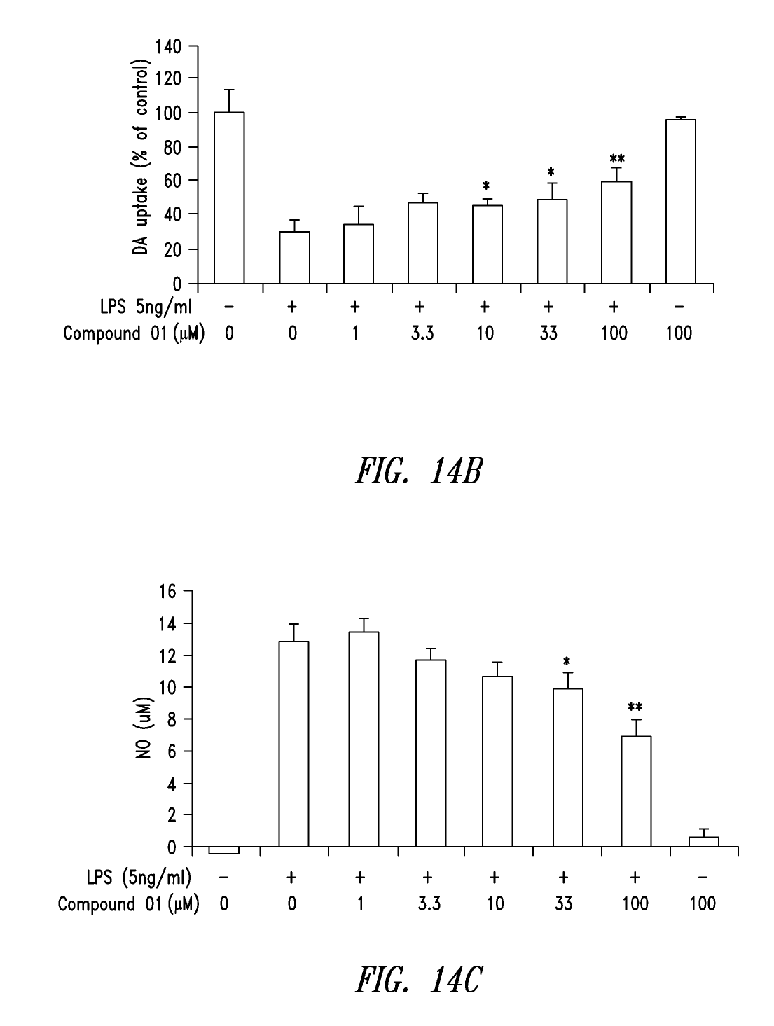

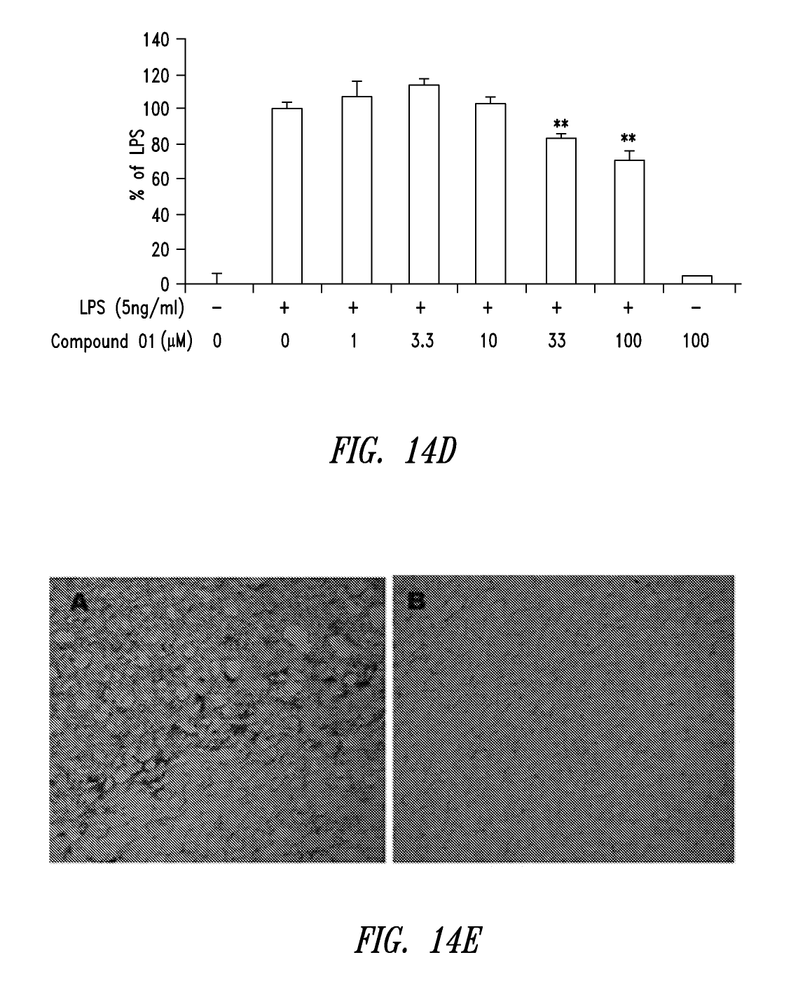

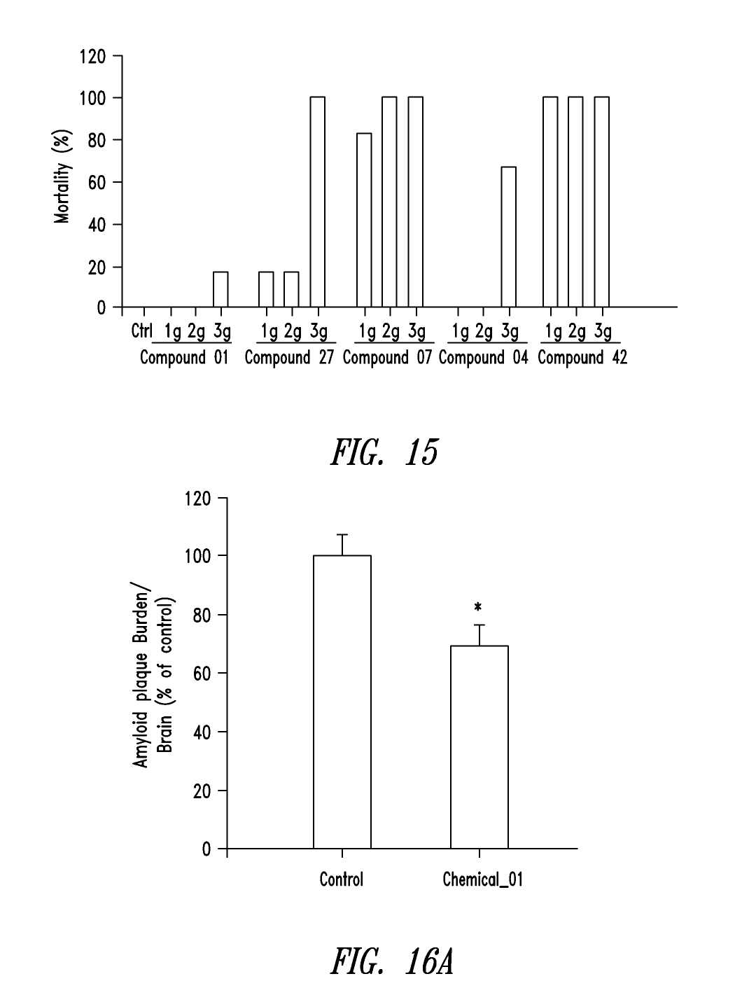

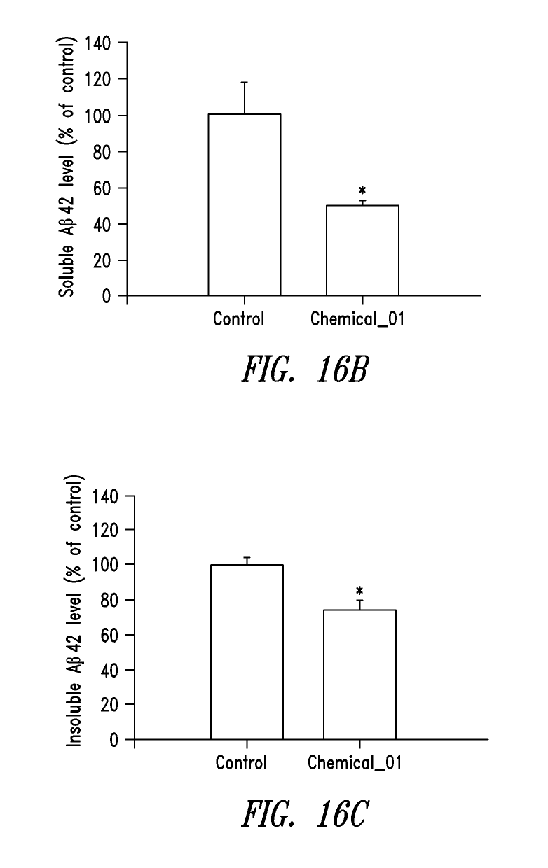

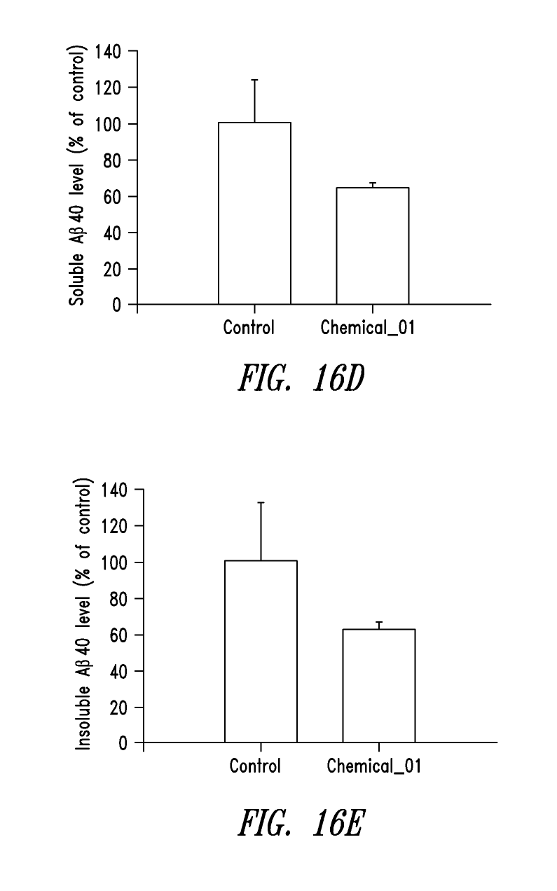

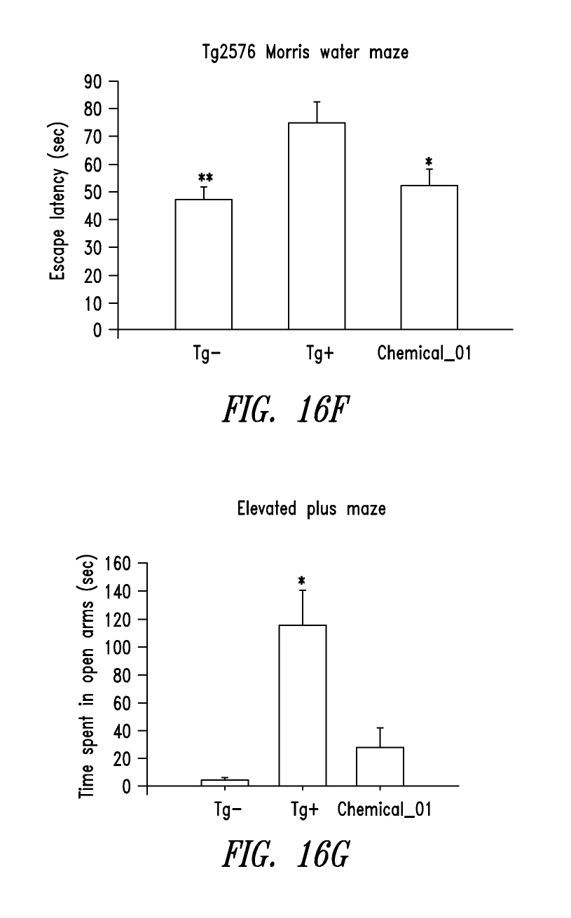

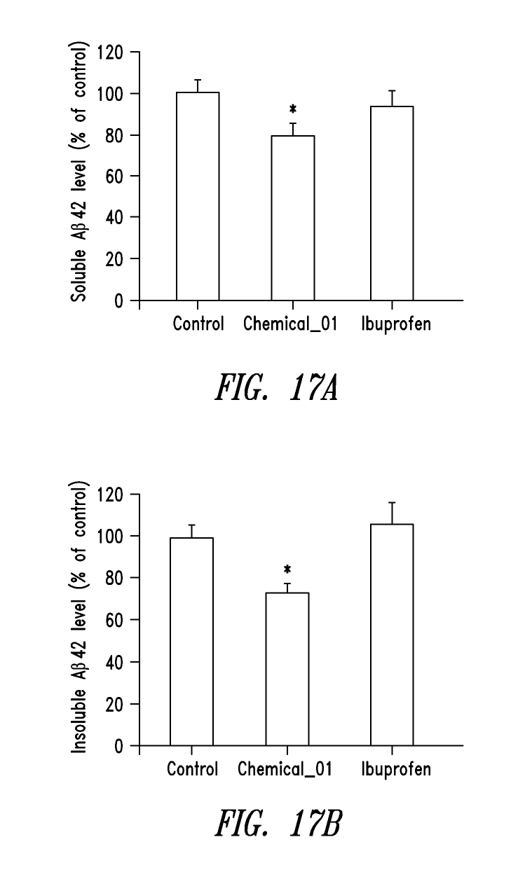

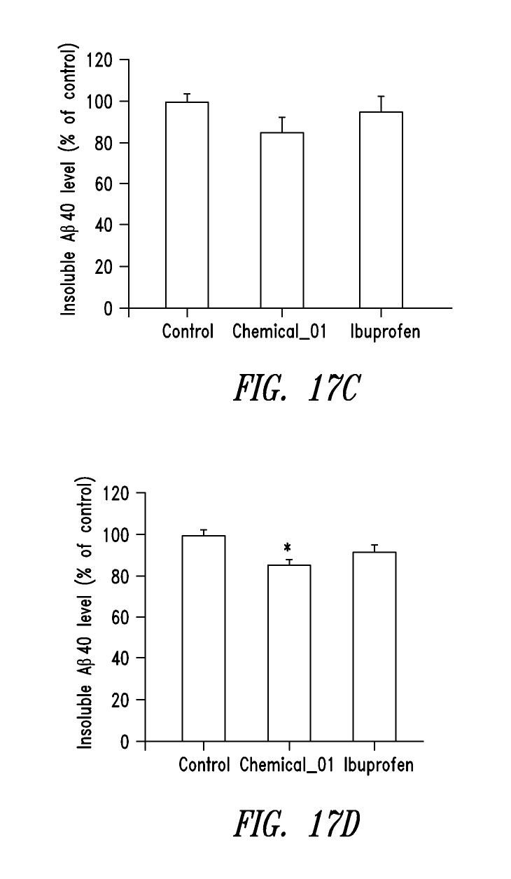

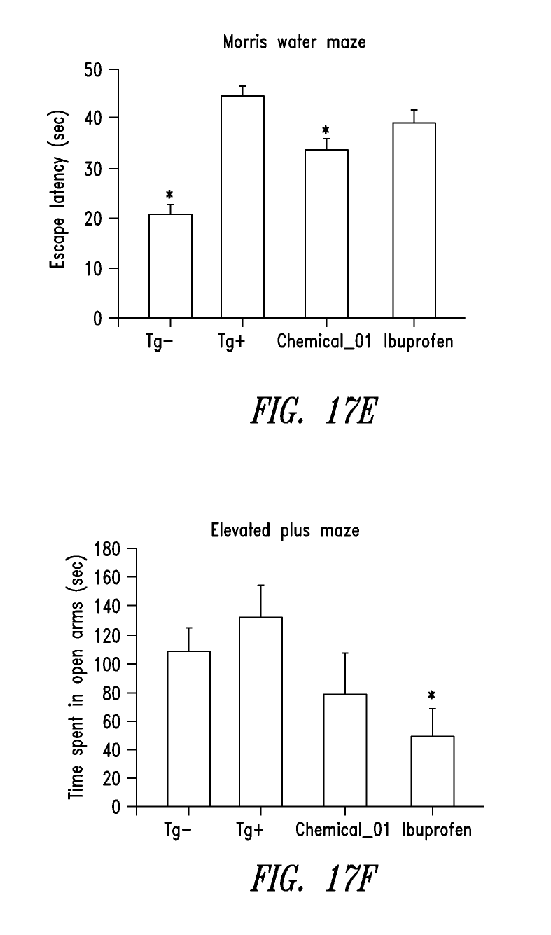

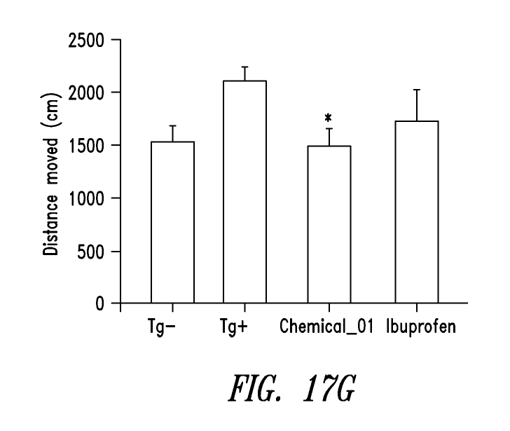

Oxidative stress was induced by exposing mixed cortical cell cultures containing neurons and glia (DIV 11-15) to 50 μM FeCl₂, a hydroxyl radical-producing transition metal via a Fenton reaction, or 10 mM DL-buthionine-[S,R]-sulfoximine (BSO), a glutathione depleting agent. Widespread neuronal death was observed 24 hours later. Concurrent administration of 2-Hydroxy-TTBA or 2-Hydroxy-TPEA nearly completely blocked free radical neurotoxicity at doses as low as 0.3 μM (FIGS. 1A & 1B). Administration of vitamin E prevented Fe²⁺-induced free radical neurotoxicity at higher doses. This implies that 2-Hydroxy-TTBA or 2-Hydroxy-TPEA is a potent neuroprotectant against oxidative stress. Neuroprotective effects of several cell necrosis inhibitors were analyzed as IC₅₀ value that showed 50% protection against Fe²⁺-induced free radical neurotoxicity (Table 1), showing that potent neuroprotective effects of BAS, CBAS, FBAS, TBAS, PBAS, MBAS, NPAA, NPPAA, 2-Hydroxy-TTBA, and 2-Hydroxy-TPEA as compared to vitamin E.

TABLE 1
BLOCKADE OF Fe²⁺-INDUCED FREE RADICAL NEUROTOXICITY
BY VITAMIN E, TROLOX, BENZYLAMINOSALICYLIC ACID
DERIVATIVES AND A TETRAFLUOROBENZYL DERIVATIVE.

| Drug | IC₅₀ (μM) |
|---|---|
| BAS | 1.24 |
| NBAS | 1.9 |
| CBAS | 0.2 |
| TBAS | 0.31 |
| MBAS | 1.42 |
| FBAS | 0.3 |
| PBAS | 0.1 |
| NPAA | 0.27 |
| NPPAA | 0.20 |
| 2-Hydroxy-TTBA | 0.11 |
| 2-Hydroxy-TPEA | 0.099 |
| Trolox | 3.34 |
| Vitamin E | 22.03 |

However, concurrent administration of 10 mM Li⁺, which was shown to attenuate apoptosis (Kang et al, 2003), did not attenuate Fe²⁺- or BSO-induced free radical neurotoxicity (FIG. 1C).

### Example 3

### Prevention of Neuronal Cell Apoptosis by Li⁺

Cortical cell cultures containing neurons and glia at 10-12 days in vitro (DIV 10-12) were exposed to 20 μM cyclosporine A (CsA) or 10 nM caliculin A (cal A). Neurons underwent widespread apoptosis 24 hr later as previously reported (McDonald et al., 1996; Ko et al., 2000). Concurrent administration of Li⁺ dose-dependently attenuated neuronal cell apoptosis at doses of 3-30 mM (FIG. 2A). Cyclosporine A-induced neuronal cell apoptosis was not attenuated by inclusion of vitamin E, 2-hydroxy-TTBA, or 2-hydroxy-TPEA (FIG. 2B). This implies that Li⁺ and the neuroprotective drugs (vitamin E, trolox, BAS, CBAS, FBAS, TBAS, PBAS, MBAS, NPAA, NPPAA, 2-Hydroxy-TTBA, and 2-Hydroxy-TPEA) selectively prevent neuronal cell apoptosis and free radical-mediated necrosis, respectively.

### Example 4

### Enhanced Prevention of Neuronal Cell Death and Motor Performance Deficit in Transgenic Mouse Model of ALS (G93A Mouse) by Combination of Both 2-hydroxy-TTBA and lithium

### (4-1) Onset of Oxidative Stress Prior to Motor Neuron Degeneration in G93A Transgenic Mice

Levels of oxidative stress were first examined in the spinal cord from wild type and transgenic mice before behavioral deficit and motor neuron degeneration were observed. Marked oxidative stress was observed in the motor neurons in the lumbar ventral horn from G93A transgenic mice compared to the wild type at ages of 8 weeks as shown by increased immunoreactivity to nitrotyrosine antibody (FIG. 3A). Fluorescence intensity of oxidized MitoTracker CM-H₂XRos was also increased in the spinal motor neurons from the transgenic mice, suggesting that the spinal motor neurons are accompanied by accumulation of protein oxidation and by free radicals. Similar levels of nitrotyrosine immunoreactivity and mitochondrial free radicals were observed in the dorsal horn neurons and white matter. Analysis of nitrotyrosine immunoreactivity showed that oxidative stress was increased up to 3-fold in the motor neurons from the transgenic mice compared to the wild type at ages of 4 weeks (FIG. 3B). Levels of nitrotyrosine were peaked to 4-fold at 8 weeks of age and then declined over 14 weeks of age. Neuronal death was slightly observed in the ventral horn from the transgenic mice at 8 weeks of age when oxidative stress was peaked (FIG. 3C). After then, neuronal death was gradually observed until the animals would die. This implies that G93AA transgenic mice produce oxidative stress selectively in the motor neurons at the early ages, which may in turn cause neurodegeneration in the lumbar ventral horn.

### (4-2) Activation of Fas-Mediated Apoptosis Signaling Pathway in G93A Transgenic Mice

Fas ligand (Fast)-mediated apoptosis plays a role in neuronal death in neurodegenerative diseases including Alzheimer's disease, Parkinson's disease, and (Morishima et al., 2001, Su et al, 2003; Hartman et al., 2002). It is conceivable to reason that the Fas signaling pathway contributes to apoptosis of the motor neurons in G93A transgenic mice. Expression and interaction of Fas and its cytoplasmic adaptor protein FADD were found to have increased in the lumbar spinal cord from the transgenic mice at 12 weeks of age compared to the wild type (FIG. 4A). Immunohistochemistry with Fas antibody revealed that levels of Fas were increased primarily in the spinal motor neurons from G93A mice (FIG. 4B). The death-inducing signaling complex was followed by activation of caspase-8, possibly through the autoproteolytic processing of procaspase-8, and caspase-3 (FIG. 4C). The active form of caspase-3 was observed primarily in the motor neurons in the lumbar spinal cord from G93A mice (FIG. 4D). This suggests that that Fas, FADD, caspase-8, and caspase-3 are activated in the spinal motor neurons to mediate subsequent neuronal apoptosis in the ALS mice at ages of 12 weeks. The activation pattern of the Fas-signaling molecules disappeared at ages of 16 weeks when most motor neurons died.

### (4-3) 2-Hydroxy-TTBA and Li⁺ Prevent Oxidative Stress and Apoptosis in Cortical Cell Cultures and in G93A Transgenic Mice, Respectively

Additional experiments were performed to examine if targeting both neuronal cell necrosis and apoptosis would result in synergic neuroprotection in G93AA transgenic mice. Oxidative stress was induced by exposure of cortical cell cultures containing neurons and glia to OH radial-producing transition metal Fe²⁺ or glutathione-depleting agent buthionine sulfoximine (BSO) that were shown to cause widespread neuronal cell necrosis within 24 hr. Fe²⁺- and BSO-induced neuronal death was completely blocked by concurrent administration of 2-hydroxy-5-(2,3,5,6-tetrafluoro-4-trifluoromethyl-benzylamino)-benzoic acid (2-hydroxy-TTBA) even at a submicromolar concentration (FIG. 5A). The neuroprotective effect of 2-hydroxy-TTBA against Fe²⁺-induced oxidative neuronal death was 220 times higher than vitamin E. The oxidative neuronal death was not attenuated by addition of lithium ion (Li⁺), a mood-stabilizing agent which was reported to selectively prevent neuronal cell apoptosis without protective effects against excitotoxic neuronal cell necrosis (Kang et al., 2003; Chuang et al., 2002). Neuronal cell apoptosis was induced by serum deprivation in neuron-rich cortical cell cultures as reported, which was prevented by addition of 5 mM Li⁺ as well as zVADfmk, a broad spectrum inhibitor of caspases (FIG. 5B). Serum deprivation-induced neuronal cell apoptosis was not attenuated by addition of 2-Hydroxy-TTBA. Additional experiments were performed to examine if Li⁺ would prevent Fas signaling pathway. Fas-FADD interaction was observed in neuron-rich cortical cell cultures deprived of serum for 8 hr, which was blocked by addition of Li⁺, but not by 2-Hydroxy-TTBA (FIG. 5C). This implies that 2-Hydroxy-TTBA and Li⁺ blocks oxidative neuronal cell necrosis and apoptosis, respectively.

G93A transgenic mice received oral administration of 2-hydroxy-TTBA (30 mg/kg/d) in the diet from 8 weeks of age. The oral administration of 2-Hydroxy-TTBA blocked nitrotyrosine, and mitochondrial free radical increased in the lumbar spinal motor neurons at 10 weeks of age compared to the wild type (FIGS. 5D & 5E). The administration of 2-Hydroxy-TTBA slightly attenuated levels of Fas, FADD, and cleaved caspase-8 and caspase-3 increased in the lumbar spinal cord from G93A transgenic mice at 12 weeks of age (FIG. 5F). Oral administration of Li⁺ (200 mg/kg/d) in the diet completely blocked the Fas pathway induced in the spinal cord from G93A mice. Thus, cell necrosis and Fas-mediated apoptosis induced in G93A mice can be prevented by oral administration of 2-hydroxy-TTBA and Li⁺.

### (4-4) 2-Hydroxy-TTBA and Lithium Synergically Delay Onset and Progression Of Motor Deficit in G93A Transgenic Mice

G93A transgenic mice revealed body weight loss down to 58% of the wild type at 18 weeks of age (FIG. 6A). The oral administration of 2-Hydroxy-TTBA or Li⁺ from 12 weeks of age alleviated weight loss to 41 and 53% of the wild type. The weight loss was further reduced to 32% by co-administration of 2-Hydroxy-TTBA and Li⁺. G93A transgenic mice fed with 2-Hydroxy-TTBA or Li⁺ in the diet showed better motor performance than the vehicle-treated control from 11 weeks to 18 weeks (FIG. 6B-6D). Onset of PaGE deficits or Rotarod deficits and mortality of ALS transgenic mice were analyzed, mean±SEM (n=13 per each group) a, p<0.01 compared to vehicle; b, p<0.05 between 2-hydroxy TTBA (or Li) alone and combination of 2-hydroxy TTBA and Li. Extension reflex, motor strength, and coordination were all improved in the transgenic ALS mice treated with either 2-Hydroxy-TTBA or Li⁺. Onset of PaGE deficiency was 104 days in vehicle-treated G93A control mice and delayed to 114.1 and 113.3 days in G93A mice treated with 2-Hydroxy-TTBA and Li⁺, respectively (Table 2).

TABLE 2
DELAYED ONSET OF MOTOR DEFICIT AND MORTALITY
OF ALS MICE TREATED WITH 2-HYDROXY-TTBA AND/OR
LITHIUM (MEAN ± SED, N = 13 PER EACH GROUP)

| | | 2-Hydroxy- | | 2-Hydroxy- |
|---|---|---|---|---|
| | Vehicle | TTBA | Li | TTBA + Li |
| Onset | 104 ± 2.70 | 114.1^{a} ± 2.02 | 113.3^{a} ± 2.28 | 127.6^{a,b} ± |
| from | | | | 7.39 |
| PaGE | | | | |
| Onset | 98.7 ± 3.30 | 112.3^{a} ± 2.89 | 114.7^{a} ± 2.23 | 121.5^{a,b} ± |
| from | | | | 4.67 |
| Rotarod | | | | |
| Mortality | 125.3 ± 2.10 | 143.8^{a} ± 2.83 | 137.2^{a} ± 2.20 | 152.1^{a,b} ± |
| | | | | 5.87 |

^{a}P < 0.01 compared with vehicle group
^{b}P < 0.05 compared with 2-Hydroxy-TTBA and lithium group

As shown in Table 2 and FIG. 7A, the onset was further delayed to 127.6 days in G93A mice treated with both 2-Hydroxy-TTBA and Li⁺. In rotarod test, onset of impaired motor performance was 98.7 days in vehicle-treated control group. The onset was 112.3 and 114.7 days in G93A mice administered with 2-Hydroxy-TTBA and Li⁺, respectively, which was further delayed to 121.5 days following co-administration of both 2-Hydroxy-TTBA and Li⁺.

Administration of 2-Hydroxy-TTBA and Li⁺ extended survival from 125.6 days to 143.8 and 137.2 days in G93A transgenic mice (Table 2, FIG. 7B). Survival was further extended to 152.1 days in G93A mice administrated with both 2-Hydroxy-TTBA and Li⁺. Finally, neuroprotective effects of 2-Hydroxy-TTBA or Li⁺ were examined in the ventral motor neurons from the lumbar spinal cord at 16 weeks of age. In the control G93A mice, motor neurons underwent widespread degeneration up to 74% (FIGS. 7C & 7D). Degeneration of motor neurons was reduced to 57 and 58% in G93A mice treated with 2-Hydroxy-TTBA and Li⁺, respectively. Neuronal loss was further reduced to 17% in G93A mice treated with combination of 2-Hydroxy-TTBA and Li⁺.

As described above, the combination of cell necrosis inhibitors and lithium of the present invention can effectively be used to treat neurological diseases or ocular diseases.

### Example 5

### Medicinal Effect Evaluation of Drug Candidates

As detailedly described in the following examples, anti-oxidant effect, anti-inflammatory effect and suppressing effect on production of beta-amyloid were evaluated with a lot of compounds.

After cerebral cortical cell was treated by 50 uM FeCl₂ to induce oxidative stress, the anti-oxidant effects of many compounds were evaluated. Results were shown as IC₅₀ (uM) (Item A of Table 3 below).

After BV-2 cell line was treated by LPS to induce production of NO, the anti-inflammatory effects of many compounds were evaluated. Results were shown as IC₅₀ (uM) (Item B of Table 3 below).

The suppressing effects of many drug candidates on production of beta-amyloid were evaluated in CHO cell line having the increased level of beta-amyloid. Results were shown as IC₅₀ (uM) (Item C of Table 3 below).

TABLE 3

| Compound | IC₅₀ (uM) | | | |
|---|---|---|---|---|
| No. | A | B | C | Compound |
| 01 | 0.099 | 24.26 | 19.60 | 2-Hydroxy-5-[2-(4-trifluoromethyl-phenyl)- |
| | | | | ethylamino]-benzoic acid |
| 02 | 1.037 | >100 | ND | 2-Hydroxy-5-phenethylamino-benzoic acid |
| 03 | 0.1 | 33.03 | ND | 2-Hydroxy-5-[2-(2-trifluoromethyl-phenyl)- |
| | | | | ethylamino]-benzoic acid |
| 04 | 0.08 | 26.14 | ND | 2-Hydroxy-5-[2-(3-trifluoromethyl-phenyl)- |
| | | | | ethylamino]-benzoic acid |
| 05 | 0.11 | 60.28 | ND | 2-Hydroxy-5-[2-(4-methoxy-phenyl)-ethylamino]- |
| | | | | benzoic acid |
| 06 | 0.27 | 82.21 | ND | 5-[2-(2,5-Difluoro-phenyl)-ethylamino]-2- |
| | | | | hydroxy-benzoic acid |
| 07 | 0.11 | 60.77 | 19.13 | 5-[2-(2-Chloro-phenyl)-ethylamino]-2-hydroxy- |
| | | | | benzoic acid |
| 08 | 0.23 | 76.55 | ND | 5-[2-(3-Chloro-phenyl)-ethylamino]-2-hydroxy- |
| | | | | benzoic acid |
| 09 | 0.14 | 78.75 | ND | 5-[2-(2-Bromo-phenyl)-ethylamino]-2-hydroxy- |
| | | | | benzoic acid |
| 10 | 0.23 | No | ND | 5-[2-(3-Bromo-phenyl)-ethylamino]-2-hydroxy- |
| | | effect | | benzoic acid |
| 11 | 0.24 | 23.94 | ND | 2-Hydroxy-5-(2-p-tolyl-ethylamino)-benzoic acid |
| 12 | 0.36 | 62.67 | ND | 5-[2-(2,6-Difluoro-phenyl)-ethylamino]-2- |
| | | | | hydroxy-benzoic acid |
| 13 | 0.27 | 94.04 | ND | 2-Hydroxy-5-[2-(2-methoxy-phenyl)-ethylamino]- |
| | | | | benzoic acid |
| 14 | 0.11 | >100 | ND | 5-[2-(4-Chloro-phenyl)-ethylamino]-2-hydroxy- |
| | | | | benzoic acid |
| 15 | 0.32 | No | ND | 2-Hydroxy-5-[2-(3-methoxy-phenyl)-ethylamino]- |
| | | effect | | benzoic acid |
| 16 | 0.10 | 22.51 | 67.5 | 2-Hydroxy-5-[2-(4-trifluoromethoxy-phenyl)- |
| | | | | ethylamino]-benzoic acid |
| 17 | 0.21 | >300 | ND | 5-[2-(2,4-Difluoro-phenyl)-ethylamino]-2- |
| | | | | hydroxy-benzoic acid |
| 18 | 0.10 | 63.25 | ND | 5-[2-(3,4-Dichloro-phenyl)-ethylamino]-2- |
| | | | | hydroxy-benzoic acid |
| 19 | 0.26 | 82.61 | ND | 5-[2-(3-Fluoro-phenyl)-ethylamino]-2-hydroxy- |
| | | | | benzoic acid |
| 20 | 0.40 | 12.45 | ND | 2-Hydroxy-5-[2-(2-nitro-phenyl)ethylamino]- |
| | | | | benzoic acid |
| 21 | 0.32 | 23.69 | 68 | 5-[2-(3,4-Difluoro-phenyl)-ethylamino]-2- |
| | | | | hydroxy-benzoic acid |
| 22 | 0.25 | 134.95 | ND | 5-[2-(3,5-Difluoro-phenyl)-ethylamino]-2- |
| | | | | hydroxy-benzoic acid |
| 23 | 0.11 | 34.93 | ND | 5-[2-(2-Fluoro-phenyl)-ethylamino]-2-hydroxy- |
| | | | | benzoic acid |
| 24 | 0.031 | 40.77 | 29.200 | 5-[2-(2,4-Dichloro-phenyl)-ethylamino]-2- |
| | | | | hydroxy-benzoic acid |
| 25 | 0.07 | 18.62 | No | 2-Hydroxy-5-(2-o-tolyl-ethylamino)-benzoic acid |
| | | | effect | |
| 26 | 0.13 | 61.73 | >100 | 5-[2-(2-Fluoro-3-trifluoromethyl-phenyl)- |
| | | | | ethylamino]-2-hydroxy-benzoic acid |
| 27 | 0.044 | 6.25 | 109.3 | 5-[2-(3,5-Bis-trifluoromethyl-phenyl)-ethylamino]- |
| | | | | 2-hydroxy-benzoic acid |
| 28 | 0.12 | 20.34 | ND | 5-[3-(4-Fluoro-phenyl)-propylamino]-2-hydroxy- |
| | | | | benzoic acid |
| 29 | 0.18 | 42.21 | ND | 5-[2-(4-Ethoxy-phenyl)-ethylamino]-2-hydroxy- |
| | | | | benzoic acid |
| 30 | 0.39 | 23.6 | No | 2-Hydroxy-5-(2-m-tolyl-ethylamino)-benzoic acid |
| | | | effect | |
| 31 | 0.25 | 4.078 | ND | 5-[2-(4-Fluoro-2-trifluoromethyl-phenyl)- |
| | | | | ethylamino]-2-hydroxy-benzoic acid |
| 32 | 0.29 | 85.43 | ND | 2-Hydroxy-5-(3-phenyl-propylamino)-benzoic |
| | | | | acid |
| 33 | 0.036 | 31.11 | >100 | 5-[2-(4-Fluoro-3-trifluoromethyl-phenyl)- |
| | | | | ethylamino]-2-hydroxy-benzoic acid |
| 34 | 0.13 | 98.84 | No | 5-[2-(3-Fluoro-5-trifluoromethyl-phenyl)- |
| | | | effect | ethylamino]-2-hydroxy-benzoic acid |
| 35 | 0.035 | 67.53 | >100 | 5-[3-(3,4-Difluoro-phenyl)-propylamino]-2- |
| | | | | hydroxy-benzoic acid |
| 36 | 0.06 | 99.8 | >100 | 5-[2-(2-Fluoro-4-trifluoromethyl-phenyl)- |
| | | | | ethylamino]-2-hydroxy-benzoic acid |
| 37 | 0.27 | >100 | ND | 5-[2-(5-Fluoro-2-trifluoromethyl-phenyl)- |
| | | | | ethylamino]-2-hydroxy-benzoic acid |
| 38 | 0.069 | 35.04 | No | 5-[2-(2-Fluoro-5-trifluoromethyl-phenyl)- |
| | | | effect | ethylamino]-2-hydroxy-benzoic acid |
| 39 | 0.31 | No | ND | 5-[2-(4-Fluoro-phenyl)-ethylamino]-2-hydroxy- |
| | | effect | | benzoic acid |
| 40 | 0.073 | >100 | No | 2-Hydroxy-5-(4-phenyl-butylamino)-benzoic acid |
| | | | effect | |
| 41 | 0.11 | 26.5 | No | 2-Hydroxy-5-(3-p-tolyl-propylamino)-benzoic |
| | | | effect | acid |
| 42 | 0.062 | 6.7 | 68.32 | 2-Hydroxy-5-[3-(4-trifluoromethyl-phenyl)- |
| | | | | propylamino]-benzoic acid |
| 43 | 0.04 | 51.78 | 110.91 | 5-[3-(3,4-Dichloro-phenyl)-propylamino]-2- |
| | | | | hydroxy-benzoic acid |
| 44 | 0.12 | 51 | ND | 5-[3-(2,4-Dichloro-phenyl)-propylamino]-2- |
| | | | | hydroxy-benzoic acid |
| 45 | 0.10 | 20.67 | ND | 5-[2-(3-Fluoro-4-trifluoromethyl-phenyl)- |
| | | | | ethylamino]-2-hydroxy-benzoic acid |
| 46 | 0.42 | No | ND | 5-[2-(3-Chloro-4-hydroxy-phenyl)-ethylamino]-2- |
| | | effect | | hydroxy-benzoic acid |
| 47 | 0.32 | 11.46 | ND | 2-Hydroxy-5-[2-(4-hydroxy-phenyl)-ethylamino-] |
| | | | | benzoic acid |

In the Table 3, A, B and C mean neuron protecting effect against Fe⁺², suppressing effect on production of NO and suppressing effect on production of beta-amyloid, respectively. The term “ND” means “Not determined.”

As shown in the Table 3, chemical 01 (2-hydroxy-5-[2-(4-trifluoromethyl-phenyl)-ethylamino]-benzoic acid) according to the present invention showed superior anti-oxidant and anti-inflammatory effects compared to other compounds having similar chemical structures. In addition, chemical 01 showed much better suppressing effect on production of beta amyloid compared to other compounds having similar chemical structures.

As shown in the Table 3, there were some compounds showing better effect in only one test, however the compounds did not good effect in the other tests needed to be a good therapeutic agent for degenerative brain disease (for example, some compounds have better anti-oxidant effect than chemical 01, but the compounds have little suppressing effect on production of beta-amyloid, which is important in treating degenerative brain disease). Furthermore, some compounds showed good therapeutic efficacy in all efficacy tests, but they showed bad safety results compared to chemical 01 like the following toxicity test.

### Example 6

### Toxicity Test of Drug Candidates

Single-dose toxicity test of compounds showing good results in the above three efficacy tests were evaluated. Results were shown in FIG. 15.

As shown in FIG. 15, LD₅₀ of chemical 01 was more than 3 g/kg, which means that chemical 01 has good safety. Chemical 07 had 0.5-1 g/kg of LD₅₀, that is, chemical 07 showed worse safety than chemical 01. Chemical 27 showed much better effect in anti-oxidant, anti-inflammatory and anti-beta amyloid production test, but chemical 27 did not show dose-dependent result in toxicity test because the compound caused sudden death of mouse at 3 g/kg of dose test. Chemicals 04 and 42 have the similar chemical structure with chemical 01, and show the similar therapeutic effects with chemical 01 in anti-oxidant and anti-inflammatory tests, but the compounds show high toxicity or dose-independent toxicity.

### Example 7

### Anti-Oxidant Effect Evaluation of Chemical 01

### (7-1) Suppressing Effect on Production of ROS in Cell

It was evaluated whether chemical 01 suppresses reactive oxygen species (ROS) increased by FeCl₂. Cortical cell cultures (DIV 11-14) were continuously exposed to 50 uM FeCl₂ alone or with inclusion of 1 uM of chemical 01. Then, the fluorescent intensity of 6-carboxy-2′,7′-dichlorofluorescin diacetate (oxidation product of DCDHF-DA) was evaluated (mean±SEM, n=3).

*, Significant difference from control (FeCl₂ alone), p<0.05 using one-way ANOVA according to Student-Neuman-Keuls' test.

Samples treated with FeCl₂ alone showed the increase of ROS after 4 hours, while chemical 01 (1 uM) suppressed the production of ROS in cell increased by FeCl₂ (FIG. 8).

### (7-2) Free Radical Scavenging Activity Evaluation

Free radical scavenging activity of chemical 01 was directly evaluated with 1,1-diphenyl-2-picrylhydrazil (DPPH, a stable free radical). Results showed that IC₅₀ of chemical 01 is 9.55 uM, which means that chemical 01 is a direct free radical scavenger.

In addition, scavenging effects of superoxide and hydroxyl radical in test tube were evaluated. Hydroxyl radical scavenging activities of 0.1 uM, 1 uM and 10 uM of chemical 01 were 13.35%, 33.33% and 71.72%, respectively. Therefore, IC₅₀ of chemical 01 was 0.97 uM. In addition, superoxide scavenging effect of chemical 01 in NADH/PMS system was 26.08 uM (IC₅₀ value), and over 100 uM (IC₅₀ value) in X/XO system.

### Example 8

### Suppressing Effect of Chemical 01 on Inflammatory Cytokines

Suppressing effects of chemical 01 in BV2 cell lines on production of IL-1β, IL-6 or TNF-α were evaluated. BV2 cells were exposed to 1 ug/ml LPS (inflammation-inducing material) with inclusion of chemical 01 at indicated doses. After 4 hours, supernatant were collected and concentrations of TNF-a were evaluated. In addition, BV2 cells were treated by the same method, and 24 hours later, supernatant were collected and concentrations of IL-1β and IL-6 were evaluated (mean±SEM, n=3). BV2 cells treated with LPS only were used as control.

*, Significant difference from control (LPS alone), p<0.05 using one-way ANOVA according to Student-Neuman-Keuls' test.

In result, 1˜100 uM of chemical 01 decreased IL-1β and IL-6 in a dose-dependent manner. 100 uM of chemical 01 decreased the amounts released into media of IL-1β, IL-6 and TNF-α by about 80%, 70%, and 70%, respectively (FIGS. 9A-C).

### Example 9

### Safety Test of Chemical 01

Conventional NSAIDs have side effects causing damages to the gastric mucous membrane. Therefore, it was evaluated whether chemical 01 having anti-inflammatory effect causes the gastric damage or not. 30, 100 or 300 mg/kg of aspirin was orally administered as control. Chemical 01 of the present invention did not cause the gastric side effect even when 1,000 mg/kg of chemical 01 was orally administered. From this result, it is believed that chemical 01 is very safe (FIG. 10A).

### Example 10

### Therapeutic Efficacy Evaluation in Tg2576 Dementia Mouse

### (10-1) Reduction Evaluation of Amyloid Plaque Burden in Tg2576 Transgenic Mice by Thioflavin-S Stain Analysis

17 month-old Tg2576 transgenic mice were fed chow alone (saline only), or containing 25 mg/kg/day of chemical 01, for 8 months before being sacrificed (9˜17 months). 18˜20 um brain cryo-sections were stained with 1% Thioflavin-S for 5 minutes and observed under fluorescence microscope system. Amyloid plaque burden/brain was quantified with MetaVue Image software (mean±SEM, n=2).

*, Significant difference from control (Tg2576 dementia mouse fed with general chow only), p<0.05 using one-way ANOVA according to Student-Neuman-Keuls' test.

In result of the quantitative analysis of amyloid burden, the treatment with chemical 01 reduced amyloid plaque burden by 39.2% compared to control (FIG. 11A).

Also, treatment with 100 mg/kg/day of chemical 01 for 6 months (from 6 to 12 month-old Tg2576) caused a significant reduction in plaque burden by 62%.

### (10-2) Reduction Evaluation of Beta Amyloid Protein by ELISA

17 month-old Tg2576 transgenic mice were fed chow alone, or containing 25 mg/kg/day of chemical 01, for 8 months before being sacrificed (9˜17 months). The level of Aβ₄₂ or Aβ₄₀ protein was quantitatively analyzed by colorimetric sandwich ELISA kit (BIOSOURCE, Camarillo, Calif.) (mean±SEM, n=2).

*, Significant difference from control (Tg2576 mouse fed with general chow only), p<0.05 using one-way ANOVA according to Student-Neuman-Keuls' test.

In result, treatment with chemical 01 reduced SDS-soluble/insoluble Aβ₄₂ or SDS-soluble/insoluble Aβ₄₀ levels compared to Tg2576 mouse fed with general chow only by 30˜50% (FIGS. 11B-E). Also, treatment with 100 mg/kg/day of chemical 01 for 6 months (from 6 to 12 months) caused a significant reduction in SDS-soluble or insoluble Aβ₄₂ levels by 40˜60% compared to Tg2576 mouse fed with general chow only.

### (10-3) Behavior Improvement Effect in Tg2576 Dementia Mouse

### (10-3-1) Morris Water Maze Test

14 month-old Tg2576 transgenic mice were fed chow alone, or containing 25 mg/kg/day of chemical 01, for 5 months (9˜14 months). After administration of 5 months, the cognitive function was analyzed by Morris water maze test. Training was performed 4 times a day (4 trials/day) for 5 days. If mouse stay on the platform for over 10 seconds, it is thought to be a success. After 5 days of experiments, the latency to find the platform was recorded and analyzed for each mouse (mean±SEM, n=2).

*, Significant difference from control (Tg2576 mouse fed with general chow only), p<0.05 using one-way ANOVA according to Student-Neuman-Keuls' test.

In result, the escape latency of treatment group with chemical 01 for 5 months were shorter than that of the control animals (FIG. 11F).

### (10-3-2) Elevated Plus Maze Test

14 month-old Tg2576 transgenic mice were fed chow alone, or containing 25 mg/kg/day of chemical 01, for 5 months (9˜14 months). After administration of 5 months, Elevated plus maze test was performed to evaluate the behavior improvement. Elevated plus maze has two open arms (30 cm×6 cm×0.5 cm) and two closed arms (30 cm×6 cm×15 cm), and also has 6 cm×6 cm of center platform. In Elevated plus maze test, mouse was carefully laid in the center with the head of the mouse toward open arm. The time that the mouse spent in the open arm was recorded for 5 minutes (mean±SEM, n=2).

*, Significant difference from control (Tg2576 mouse fed with general chow only), p<0.05 using one-way ANOVA according to Student-Neuman-Keuls' test.

In result, the treatment with 25 mg/kg/day of chemical 01 decreased the time for mouse to stay in the open arm compared to the control animals (FIG. 11G).

### Example 11

### Efficacy Test of Chemical 01 in APP_{SWE}/PS1_{deltaE9} Double Transgenic Dementia Mice

### (11-1) Reduction Evaluation of Amyloid Plaque Burden by Thioflavin-S Stain Analysis

The treatment with 25 mg/kg/day of chemical 01 for 7 months (from 3.5 to 10.5 month-old APP/PS1) caused a significant 53% reduction in amyloid plaque burden compared to APP/PS1 dementia mouse fed with general chow only. In addition, the treatment with 25 mg/kg/day of chemical 01 for 4 months (from 8.5 to 12.5 month-old APP/PS1) caused a significant 49.3% reduction in amyloid plaque burden compared to APP/PS1 dementia mouse fed with general chow only.

### (11-2) Reduction Evaluation of Beta Amyloid Protein by AB ELISA Analysis

17.5 month-old APP/PS1 transgenic dementia mice were fed chow alone, or containing 25 mg/kg/day of chemical 01 or 62.5 mg/kg/day of ibuprofen, for 14.5 months before being sacrificed (3˜17.5 months). After administration of drug for 14.5 months, Aβ₄₀ or Aβ₄₂ protein level was analyzed by colorimetric sandwich ELISA kit (BIOSOURCE, Camarillo, Calif.) (mean±SEM, n=3-5).

*, Significant difference from control (APP/PS1 mouse fed with general chow only), p<0.05 using one-way ANOVA according to Student-Neuman-Keuls' test.

In result, treatment with 25 mg/kg/day of chemical 01 reduced SDS-soluble/insoluble Aβ₄₀ or SDS-soluble/insoluble Aβ₄₂ levels by 15˜25% compared to APP/PS1 mouse fed with general chow only (FIGS. 12A-D).

In addition, treatment with 25 mg/kg/day of chemical 01 for 7 months (from 3.5 to 10.5 month-old APP/PS1) caused a significant 42% reduction in SDS-insoluble Aβ₄₀ levels compared to APP/PS1 mouse fed with general chow only. Treatment with 25 mg/kg/day of chemical 01 for 4 months (from 8.5 to 12.5 month-old APP/PS1) caused a significant 27% reduction in SDS-insoluble Aβ₄₀ levels.

Also, treatment with 25 mg/kg/day of chemical 01 for 5 days in 11.5 month-old APP/PS1 mouse caused a significant 44% reduction in SDS-insoluble Aβ₄₂ levels in plasma and a significant 38% reduction in SDS-insoluble Aβ₄₀ levels in plasma compared to APP/PS1 mouse fed with general chow only.

### (11-3) Behavior Improvement Effect in APP/PS1 Dementia Mouse

### (11-3-1) Morris Water Maze Test

17.5 month-old APP/PS1 transgenic mice were fed chow containing 25 mg/kg/day of chemical 01 or 62.5 mg/kg/day of ibuprofen, for 14.5 months (3˜17.5 months). After administration of 14.5 months, the cognitive function was analyzed by Morris water maze test like example 10-3-1 (mean±SEM, n=3-5).

*, Significant difference from control (APP/PS1 mouse fed with general chow only), p<0.05 using one-way ANOVA according to Student-Neuman-Keuls' test.

In result, the escape latency of treatment group with chemical 01 for 14.5 months were shorter than that of 17.5 month-old APP/PS1 dementia mouse fed with general chow only (FIG. 12E).

### (11-3-2) Elevated Plus Maze Test

17.5 month-old APP/PS1 transgenic mice were fed chow alone, or containing 25 mg/kg/day of chemical 01 or 62.5 mg/kg/day of ibuprofen, for 14.5 months (3˜17.5 months). After administration of 14.5 months, Elevated plus maze test was performed to evaluate the behavior improvement like example 10-3-2 (mean±SEM, n=3-5).

*, Significant difference from control (APP/PS1 mouse fed with general chow only), p<0.05 using one-way ANOVA according to Student-Neuman-Keuls' test.

In result, the treatment with 25 mg/kg/day of chemical 01 for 14.5 months decreased the time for mouse to stay in the open arm compared to 17.5 month-old APP/PS1 fed with general chow only (FIG. 12F).

### (11-3-3) Open Field Test

17.5 month-old APP/PS1 transgenic mice were fed chow alone, or containing 25 mg/kg/day of chemical 01 or 62.5 mg/kg/day of ibuprofen, for 14.5 months (3˜17.5 months). After administration of drug for 14.5 months, Open field activity test was performed to evaluate locomotor activity and exploratory behavior. 4×4 of scale marks were drawn in white acryl box having a size of 60 cm×60 cm×35 cm (width×length×height) to make sixteen squares. Four squares in the center became center zone and twelve squares around the center zone became peripary zone. Mice were laid into transparent acryl tube (diameter: about 3.5 inches) laid at the left corner of the acryl box for 30 seconds. After 30 seconds, acryl tube was removed to make mouse free. Experiment was once performed for each mouse for 5 minutes. The distance traveled in the open field was recorded (mean±SEM, n=3-5).

*, Significant difference from control (APP/PS1 mouse fed with general chow only), p<0.05 using one-way ANOVA according to Student-Neuman-Keuls' test.

In result, the treatment with 25 mg/kg/day of chemical 01 for 14.5 months reduced significantly open field activity compared to 17.5 month-old APP/PS1 dementia mouse fed with general chow only (FIG. 12G).

### Example 12

### Efficacy Test of Chemical 01 in G93A, ALS Animal Model

### (12-1) Improvement of Motor Performance Ability and Increase of Survival Rate

G93A (Glycine Alanine) mouse having similar pathophysiological characteristics with ALS human patient was used to evaluate therapeutic effect of drug in ALS (amyotrophic lateral sclerosis), one of main degenerative brain diseases. It was analyzed whether treatment with 5 mg/kg/day or 20 mg/kg/day of chemical 01 improved motor performance ability or not.

In result, G93A transgenic mice fed with chow and chemical 01 showed better motor performance than control fed with general chow only from 14 weeks to 17 weeks (FIGS. 13A-B).

In PaGE test and Rotarod test, onset and mortality were analyzed for four mice per each group (FIGS. 13C-D). As shown in FIG. 13C, the onset was delayed to 125.67 days in G93A mice treated with 5 mg/kg/day or 20 mg/kg/day of chemical 01 compared to 103.5 days of G93A mouse fed with general chow only. In survival ability, G93A mouse fed with chow only, G93A mouse treated with 5 mg/kg/day of chemical 01 and G93A mouse treated with 20 mg/kg/day of chemical 01 survived for 131.57 days, 153.13 days, and 150.73 days, respectively. Treatment with chemical 01 extended survival rate of G93A mouse (FIG. 13D).

### (12-2) Reduction of Oxidative Toxicity

Reactive oxygen species (ROS) produced in progress of ALS disease was evaluated in G93A mouse. Oxidative stress was observed in the motor neurons in the lumbar ventral horn from G93A transgenic mice compared to the wild type at ages of 8 weeks by nitrotyrosine immuno-staining method. Analysis of nitrotyrosine immunoreactivity showed that oxidative stress was increased up to over 4-fold in the motor neurons from the transgenic mice compared to the wild type at ages of 10 weeks, and treatment with some amount of chemical 01 decreased fluorescence intensity of nitrotyrosine, which means that chemical 01 of the present invention significantly reduced oxidative toxicity (FIG. 13E).

### (12-3) Reduction of Microglia Number and Microglia Activation

Number and activation degree of microglia (a marker of inflammation in brain disease animal model) expressed in the lumbar ventral horn of G93A mouse were evaluated with TOMATO Lectin dye. In G93A mouse, number of microglia was increased and microglia was more activated compared to the wild type mouse. Treatment with 5 mg/kg/day or 20 mg/kg/day of chemical 01 decreased the increase of number and activation degree of microglia (FIG. 13F).

### (12-4) Reduction of Cytokine

Lumbar segments of 16 week-old G93A mice fed with general chow only, and 16 week-old G93A mice fed with chow containing 5 mg/kg/day or 20 mg/kg/day of chemical 01 were extracted and their RNA were separated. The mRNA expression degrees of TNF-a and IL-16, cytokines inducing inflammation, were evaluated through RT-PCT. In result, administration of chemical 01 effectively reduced inflammatory cytokines (FIG. 13G).

### Example 13

### Efficacy Test of Chemical 01 in Cell Culture Model and Animal Model of Parkinson'S Disease

### (13-1) Efficacy Test of Chemical 01 by Using Nerve Cell Death by MPP+

MPP+, a complex inhibitor of mitochondria, is known to inhibit metabolism of mitochondria and participate in production of oxygen. Therefore, MPP+ has been used to create cell culture model or animal model of Parkinson's disease (Dauer W and Przedborski S, Neuron. 2003; 39(6): 889-909).

Cerebral cortical cell cultures (DIV 13˜15) were continuously exposed to 50 uM MPP+ alone or with inclusion of 0.03-3 uM of chemical 01. After 24 hours, activity of LDH released outside cell was evaluated to quantify death of nerve cell (mean±SEM, n=4 culture well/each group).

*, Significant difference from control (MPP+ alone), p<0.05 using one-way ANOVA according to Student-Neuman-Keuls' test.

In result, chemical 01 reduced nerve cell death caused by MPP+ in a dose-dependent manner. IC₅₀ of chemical 01 was 0.057 uM, which is thought to be very efficacious (FIG. 14A).

### (13-2) Efficacy Test of Chemical 01 by Using Nerve Cell Death by LPS

Mesencephalic nerve cell cultures were pre-treated with 5 ng/ml of LPS for 30 minutes to make a cull culture model of Parkinson's disease, and then 0-100 uM of chemical 01 were added. 7 days after administration, [³H]DA uptake was evaluated to quantify the death of the nerve cell (mean±SEM, n=4 culture well/each group).

* and **, Significant difference from control (LPS alone), p<0.01 and p<0.001, respectively, using one-way ANOVA according to Student-Neuman-Keuls' test.

In result, chemical 01 reduced the dopaminergic nerve cell death caused by LPS in a dose-dependent manner (FIG. 14B).

### (13-3) Efficacy Test of Chemical 01 by Using Inflammation by LPS

Mesencephalic nerve cell cultures were pre-treated with 5 ng/ml of LPS for 30 minutes to make a cull culture model of Parkinson's disease, and then 0-100 uM of chemical 01 were added. The level of NO was evaluated 24 hours after administration of chemical 01, and the level of TNF-a was evaluated 3 hours after administration of chemical 01 (mean±SEM, n=7-12 culture well/each group).

* and **, Significant difference from control (LPS alone), p<0.01 and

p<0.001, respectively, using one-way ANOVA according to Student-Neuman-Keuls' test.

In result, chemical 01 reduced the increases of NO and TNF-a caused by LPS in a dose-dependent manner (FIGS. 14C-D).

### (13-4) Suppressing Effect of Chemical 01 on the Activation of Microglia in Parkinson's Disease Animal Model

MPTP (40 mg/kg) was subcutaneously injected into C57/BL6 mice (male/8 week-old). Some of the mice were administered with 50 mg/kg of chemical 01 through intraperitoneal injection 30 minutes before the injection of MPTP everyday. Two days later, brain tissue was extracted and immunostained with CD11b. After reaction, DAB (diaminobenzidine) was used for chromograph, and then the activation degree of microglia (a marker of inflammation in brain disease model) was evaluated with optical microscope (FIG. 14E).

In result, the activity of microglia caused by MPTP was decreased by the administration of chemical 01.

### Example 14

### The Effect of Chemical 01-08 in Cho Cells

Cells were treated with increasing concentrations of various chemicals in Chinese hamster ovary (CHO) cells stably transfected with human wild APP₆₉₅ and the PS1_{ΔExon9}, and analyzed Aβ₄₂ levels in culture medium by colorimetric sandwich ELISA kit (BIOSOURCE, Camarillo, Calif.). In CHO cells, IC₅₀ of Chemical_{—}01-08 on Aβ₄₂ lowering effect was 20-100 uM (Table 4).

TABLE 4
ANALYSIS OF AB₄₂ FROM CULTURED CELLS BY ELISA

| Drug no. | Aβ₄₂ lowering effect (IC₅₀, μM) |
|---|---|
| Chemical_01 | 19.6 |
| Chemical_02 | 19.13 |
| Chemical_03 | 67.50 |
| Chemical_04 | 68.00 |
| Chemical_05 | 29.20 |
| Chemical_06 | 109.30 |
| Chemical_07 | 68.32 |
| Chemical_08 | 110.91 |

### Example 15

### The Effect of Chemical_{—}01 in Tg2576 Transgenic Mice

### (15-1) Reduction of Amyloid Plaque Burden in Tg2576 Transgenic Mice

17 month-old Tg2576 transgenic mice were fed chow alone (saline only), or containing 25 mg/kg/day of chemical_{—}01, for 8 months before being sacrificed (9˜17 month). 18˜20 μm brain sections stained 1% Thioflavin-S for 5 min and observed under fluorescence microscope system. As a quantitative analysis of amyloid burden, treatment with 25 mg/kg/d of chemical_{—}01 reduced significantly amyloid plaque burden (FIG. 16A).

Also, treatment with 100 mg/kg/d of chemical_{—}01 for 6 months (from 6 to 12 months) caused a significant 62% reduction in plaque burden (data not shown).

### (15-2) Reduction of SDS-Soluble/Insoluble Aα₄₂ or Aβ₄₀ Levels in Drug-Treated Tg2576 Transgenic Mice

17 month-old Tg2576 transgenic mice were fed chow alone (saline only), or containing 25 mg/kg/day of chemical_{—}01, for 8 months before being sacrificed (9˜17 month). SDS-soluble/insoluble Aβ₄₂ or Aβ₄₀ levels were analyzed by colorimetric sandwich ELISA kit (BIOSOURCE, Camarillo, Calif.).

Treatment group with 25 mg/kg/d of chemical_{—}01 reduced SDS-soluble/insoluble Aβ₄₂ and Aβ₄₀ levels (FIGS. 16B-E).

Also, treatment with 100 mg/kg/d of chemical_{—}01 for 6 months (from 6 to 12 months) caused a significant 40˜60% reduction in SDS-soluble/insoluble Aβ₄₂ levels (data not shown).

### (15-3) Progression of Impaired Cognitive Function in Drug-Treated Tg2576 Transgenic Mice

14 month-old Tg2576 transgenic mice were fed chow alone (saline only), or containing 25 mg/kg/day of chemical_{—}01, for 5 months (9˜14 month).

The cognitive function was analyzed by Morris water maze test. Mice ware trained to perform a hidden platform task in the Morris water maze. The latency to find the platform was recorded for each mouse.

The escape latency of treatment group with 25 mg/kg/d of chemical_{—}01 were shorter than that of the control animals (FIG. 16F).

### (15-4) Reduction of Anxiety in Drug-Treated Tg2576 Transgenic Mice

14 month-old Tg2576 transgenic mice were fed chow alone (saline only), or containing 25 mg/kg/day of chemical_{—}01, for 5 months (9˜14 month).

The anxiety function was analyzed by elevated plus maze test. The time spent in the open arm was recorded in the elevated plus maze.

The treatment group with 25 mg/kg/d of chemical_{—}01 visited the open arm less frequently and spent less time there than the control animals (Tg+) (FIG. 17G).

### Example 16

### The Effect of Chemical 01 in APP_{SWE}/PS1_{deltaE9} Transgenic Mice

### (16-1) Reduction of Amyloid Plaque Burden in APP_{swe}/PS1_{deltaE9} Double Transgenic Mice

10.5 month-old APP_{swe}/PS1_{deltaE9} double transgenic mice were fed chow alone (saline only), or containing 25 mg/kg/day of chemical_{—}01, for 7.5 months before being sacrificed (3˜10.5 month). 18-20 μm brain sections stained 1% Thioflavin-S for 5 min and observed under fluorescence microscope system. As a quantitative analysis of amyloid burden, treatment with 25 mg/kg/d of chemical_{—}01 caused a significant 53.4% reduction in plaque burden (data not shown). Also, the treatment with 25 mg/kg/d of chemical_{—}01 for 7 months (from 8.5 to 12.5 months) caused a significant 49.3% reduction in plaque burden (data not shown).

### (16-2) Reduction of SDS-Soluble/Insoluble Aβ₄₀ or Aβ₄₂ Levels in APP_{swe}/PS1_{deltaE9} Double Transgenic Mice

17.5 month-old APP_{swe}/PS1_{deltaE9} double transgenic mice were fed chow alone (saline only), or containing 25 mg/kg/day of chemical_{—}01 or 62.5 mg/kg/day of ibuprofen, for 14.5 months before being sacrificed (3˜17.5 month). SDS-soluble/insoluble Aβ₄₀ or Aβ₄₂ levels were analyzed by colorimetric sandwich ELISA kit (BIOSOURCE, Camarillo, Calif.).

Treatment group with 25 mg/kg/d of chemical_{—}01 reduced SDS-soluble/insoluble Aβ₄₀ or Aβ₄₂ levels, in contrast ibuprofen did not reduce them (FIGS. 17A-D).

In further, treatment with 25 mg/kg/d of chemical_{—}01 for 7 months (from 3.5 to 10.5 months) caused a significant 42% reduction in SDS-insoluble Aβ₄₀ levels (data not shown). Also, treatment with 25 mg/kg/d of chemical_{—}01 for 5 days in 11.5 months caused a significant 40% reduction in SDS-insoluble Aβ₄₀/Aβ₄₂ levels in plasma (data not shown).

### (16-3) Progression of Impaired Cognitive Function in APP_{swe}/PS1_{deltaE9} Double Transgenic Mice

17.5 month-old APP_{swe}/PS1_{deltaE9} double transgenic mice were fed chow alone (saline only), or containing 25 mg/kg/day of chemical_{—}01, for 14 months (3˜17.5 month).

The cognitive function was analyzed by Morris water maze test. Mice ware trained to perform a hidden platform task in the Morris water maze. The latency to find the platform was recorded for each mouse.

The escape latency of treatment group with 25 mg/kg/d of chemical_{—}01 were shorter than that of the control animals (FIG. 17E) but treatment with ibuprofen did not improved cognitive function.

### (16-4) Reduction of Anxiety in APP_{swe}/PS1_{deltaE9} Double Transgenic Mice

17.5 month-old APP_{swe}/PS1_{deltaE9} double transgenic mice were fed chow alone (saline only), or containing 25 mg/kg/day of chemical_{—}01, for 14 months (3˜17.5 month).

The anxiety function was analyzed by elevated plus maze test. The time spent in the open arm was recorded in the elevated plus maze.

The treatment group with 25 mg/kg/d of chemical_{—}01 visited the open arm less frequently and spent less time there than the control animals (Tg+) (FIG. 17F).

### (16-5) Reduction of Open Field Activity in Drug-Treated APP_{swe}/PS1_{deltaE9} Double Transgenic Mice

17.5 month-old APP_{swe}/PS1_{deltaE9} double transgenic mice were fed chow alone (saline only), or containing 25 mg/kg/day of chemical_{—}01, for 14 months (3˜17.5 month).

Open field activity was used to evaluate locomotor activity and exploratory behavior. The distance traveled in the open field was recorded and performance was analyzed after the testing took place.

The treatment group with 25 mg/kg/d of chemical_{—}01 reduced significantly open field activity but there was non-significant trend for open field activity in treatment with ibuprofen (FIG. 17G).

Examples of concrete diseases applicable with the combination of the present invention are described as follows. However, the scope of the present invention is not limited to the diseases described below.

### Application Example 1

### Lou Gehrig Disease (or Amyotrophic Lateral Sclerosis)

Lou Gehrig Disease is named amyotrophic lateral sclerosis (ALS) or motor neuron disease, and the progressive degeneration of upper and lower motor neurons is the pathological hallmark of this disease. Many hypotheses have been put forward to account for the selective death of motor neurons in ALS.

ALS patients show increased levels of extracellular glutamate and loss of glutamate transporter GLT-1. Administration of glutamate receptor agonists into the spinal cord mimicked pathological changes in the spinal cord of ALS patients (Rothstein J D et al., 1995; Ikonomidou C et al., 1996).

The recent discovery of mutations affecting the superoxide dismutase (SOD) gene has given impetus to research on the role of oxidative stress in the pathogenesis of familial ALS (Robberecht W, 2000). Nonetheless, evidence shows that there is abnormal oxidative damage to proteins in postmortem samples from ALS patients. Post-mortem studies in ALS patients demonstrated increased nitrotyrosine immunoreactivity and total protein carbonylation in spinal motor neurons (Abe K et al., 1995; Shaw P J et al., 1995).

Recently, interest has been generated by the possibility that a mechanism of programmed cell death, termed apoptosis, is responsible for the motor neuron degeneration in ALS (Sathasivam S et al., 2001).

Therefore, a combination of the present invention can be used as therapeutic drugs for ALS.

Also, 2-hydroxy-alkylamino-benzoic acid derivatives according to the present invention can be effectively used as a therapeutic drug for ALS.

### Application Example 2

### Alzheimer's Disease

Alzheimer's disease is the most common form of adult onset dementia. Alzheimer's disease is characterized as the presence of the neurofibrillary tangles (NFT), amyloid plaques and neuronal death.

The direct evidence supporting increased oxidative stress in AD is: (1) increased brain Fe, Al, and Hg in AD, capable of stimulating free radical generation; (2) increased lipid peroxidation in AD brain; (3) increased protein and DNA oxidation in the AD brain (Olanow C W et al., 1994; Markesbery W R, 1997).

Also, a low- to moderate-affinity uncompetitive N-methyl-D-aspartate receptor antagonist, memantine, has been shown to improve learning and memory in several pharmacological models of AD, suggesting that NMDA antagonist has therapeutic potential in AD (Minkeviciene R et al., 2004).

Several studies have shown the activation of caspase-3 or caspase-9 during apoptosis in Alzheimer's disease (Kang H J et al., 2005; Chong Z Z et al., 2005).

Therefore, the combination of the present invention showing protective effect against cell necrosis and apoptosis can be used as therapeutic drugs for Alzheimer's disease.

Also, 2-hydroxy-alkylamino-benzoic acid derivatives showing anti-oxidant and anti-inflammatory effects according to the present invention can be effectively used as a therapeutic drug for Alzheimer's disease.

### Application Example 3

### Parkinson's Disease (PD)

Parkinson's Disease (PD), the prototypic movement disorder, is characterized clinically by tremor, rigidity, bradykinesia and postural instability and diagnosed pathologically by a selective death of dopaminergic neurons in the substantia nigra.

In PD patients, oxidative stress has been proved as a main mechanism of dopaminergic neuronal cell death, and the increased production of lipid peroxidation and ROS and the decreased GSH contents has been reported, suggesting that oxidative stress plays a causative role in neuronal death in PD (Sriram K et al., 1997; Wu D C et al., 2003).

Also, several antagonists of NMDA receptors protect dopaminergic neurons from the dopaminergic neurotoxin MPTP (1-methyl-4-phenyl-1,2,3,6-tetrahydropyridine) (Brouillet E and Beal M F, 1993).

Many in vivo studies have shown that there is some evidence for the occurrence of apoptosis in the Parkinsonian substantia. For example, there is increased neuronal expression of caspases (Hartmann A et al., 2000 and 2001) in animal model of Parkinson's Disease, suggesting that these cells are undergoing apoptosis.

Therefore, the combination of the present invention showing protective effect against cell necrosis and apoptosis can be used as therapeutic drugs for Parkinson's disease.

Also, 2-hydroxy-alkylamino-benzoic acid derivatives according to the present invention can be effectively used as a therapeutic drug for Parkinson's disease.

### Application Example 4

### Huntington's Disease (HD)

Huntington's disease (HD) is a progressive neurodegenerative disease predominantly affecting small- and medium-sized interneurons in the striata.

These pathological features of HD are observed in vivo and in vitro following administration of NMDA receptor agonists, raising the possibility that NMDA receptor-mediated neurotoxicity contributes to selective neuronal death in HD (Koh J Y et al., 1986; Beal M F et al., 1986).

Strialtal projection neurons are highly vulnerable to apoptosis in HD. Recent data have shown that there is increased expression of cytochrome C and caspase-9 in HD (Kiechle T et al., 2002) and also many TUNEL-positive cells accompanied with weak caspase-3 immunoreactivity in severely affected HD brains, suggests that neuronal apoptosis plays a role in HD (V is J C et al., 2005).

Since evidence is being accumulated that oxidative stress, such as mitochondrial dysfunction and generation of ROS, causes neuronal death observed in HD, it is possible that the drugs inhibiting ROS are used for therapy of HD (Perez-Severiano F et al., 2003; Rosenstock T R et al., 2004).

Therefore, the combination of the present invention showing protective effect against cell necrosis and apoptosis can be used as therapeutic drugs for HD.

Also, 2-hydroxy-alkylamino-benzoic acid derivatives according to the present invention can be effectively used as a therapeutic drug for Huntington's disease.

### Application Example 5

### Stroke

Stroke is a sudden problem affecting the blood vessels of the brain, and interrupted blood supply to brain or stroke induces neuronal death primarily through overactivation of glutamate receptor. It has been well documented that NMDA receptor antagonists decrease the neuronal cell death by ischemic stroke [Simon R P et al., 1984].

Also, when brain hypoxic ischemia occurs, mitochondrial electron transport system can be injured, so ROS production increases. Increased production of ROS is capable of causing neuronal death through lipid peroxidation, DNA oxidation or protein oxidation. Some antioxidants showed efficiency in animal models of hypoxic ischemia (Yamaguchi T et al., 1998).

It has also been reported that apoptosis is main mechanism of neuronal death following hypoxic ischemia. Markers of neuronal apoptotic cell death were observed in regions with hypoxic ischemia (Hu X et al., 2002).

Therefore, the combination of the present invention showing protective effect against cell necrosis and apoptosis can be used as therapeutic drugs for stroke.

### Application Example 6

### Traumatic Brain Injury (TBI) and Traumatic Spinal Cord Injury (TSCI)

Excitotoxins are closely related to the degeneration of neuronal cells following traumatic brain injury (TBI) and traumatic spinal cord injury (TSCI). It has been reported that NMDA receptor antagonists decrease the neuronal death following TBI and TSCI (Faden Al et al., 1988; Okiyama K et al., 1997).

Traumatic injuries to spinal cord or brain cause tissue damage, in part by initiating reactive biochemical changes. Numerous studies have provided considerable support for lipid peroxidation reactions, Ca²⁺ influx, and disruption of membrane in the TBI and TSCI and anti-oxidants also inhibit tissue damage following TBI and TSCI (Faden Al and Salzman S, 1992; Juurlink B H and Paterson P G, 1998).

Recent evidence provides that special caspases expression can be found in the TBI and TSCI and also inhibition of caspase has therapeutic in the treatment of TBI and TSCI (Clark R S et al., 2000; Li M et al., 2000; Keane R W et al., 2001).

Therefore, the combination of the present invention showing protective effect against cell necrosis and apoptosis can be used as therapeutic drugs for traumatic Spinal Cord injuries.

### Application Example 7

### Glaucoma, Diabetic Retinopathy or Macular Degeneration

In glaucoma, the increased intraocular pressure blocks blood flow into retina and causes retinal hypoxia. The degeneration of retina cells can also occur through excitotoxicity and the increased generation of reactive oxygen species during reperfusion and also hypoxia lead to apoptosis (Osborne N N et al., 1999; Hartwick A T, 2001; Nickells R W, 1999; Tempestini A et al., 2003). Recent studies have demonstrated that antioxidants may be a new therapeutic tool to prevent ocular diseases (Neufeld A H et al., 2002; Richer S et al., 2004).

Also, increasing amounts of evidence suggest that neurodegeneration in diabetic retinopathy and macular degeneration relates to excitotoxicity, oxidative damage and apoptosis (Lieth E et al., 2000; Moor P et al., 2001; Simonelli F et al., 2002; Barber A J, 2003; Joussen A M et al., 2003).

Therefore, the combination of the present invention showing protective effect against cell necrosis and apoptosis can be used as therapeutic drugs for ocular diseases such as glaucoma, diabetic retinopathy and macular degeneration.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

What is claimed is:

1. A method for treating neuronal death in neurological disease or ocular disease in a human or animal, which comprises administering to the human or animal in need thereof a therapeutically effective amount of a cell necrosis inhibitor and concomitantly or sequentially administering a therapeutically effective amount of lithium or a pharmaceutically acceptable salt thereof.

2. The method of claim 1, wherein the neurological disease is selected from amyotrophic lateral sclerosis (ALS, Lou Gehrig's disease), spinal muscular atrophy, Alzheimer's disease, Parkinson's disease, Huntington's disease, stroke, traumatic brain injury, and spinal cord injury.

3. The method of claim 1, wherein the ocular disease is selected from glaucoma, diabetic retinopathy and macular degeneration.

4. The method of claim 1, wherein the cell necrosis inhibitor is at least one selected from:
(i) a benzylaminosalicylic acid derivative of the following formula (I) or pharmaceutically acceptable salts thereof and
(ii) a tetrafluorobenzyl derivative of the following formula (II) or pharmaceutically acceptable salts thereof: 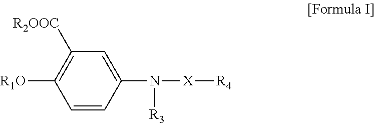
wherein,
X is CO, SO₂ or (CH₂)ₙ, wherein n is an integer from 1 to 5;
R₁ is hydrogen, alkyl or alkanoyl;
R₂ is hydrogen or alkyl;
R₃ is hydrogen or an acetoxy group; and
R₄ is a phenyl group which is unsubstituted or substituted with one or more of nitro, halogen, haloalkyl, and C₁-C₅ alkoxy; 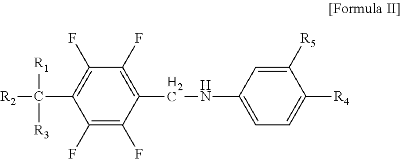
wherein,
R₁, R₂ and R₃ are independently hydrogen or halogen;
R₄ is hydroxy, alkyl, alkoxy, halogen, alkoxy substituted with halogen, alkanoyloxy or nitro; and
R₅ is carboxyl acid, ester having C₁-C₄ alkyl, carboxyamide, sulfonic acid, halogen or nitro.

5. The method of claim 4, wherein the benzylaminosalicylic acid derivative is at least one selected from:
5-benzylaminosalicylic acid,
5-(4-nitrobenzyl)aminosalicylic acid,
5-(4-chlorobenzyl)aminosalicylic acid,
5-(4-trifluoromethylbenzyl)aminosalicylic acid,
5-(4-fluorobenzyl)aminosalicylic acid,
5-(4-methoxybenzyl)aminosalicylic acid,
5-(4-pentafluorobenzyl)aminosalicylic acid,
5-(4-nitrobenzyl)amino-2-hydroxy ethyl benzoate,
5-(4-nitrobenzyl)-N-acetylamino-2-hydroxy ethylbenzoate,
5-(4-nitrobenzyl)-N-acetylamino-2-acetoxy ethylbenzoate,
5-(4-nitrobenzoyl)aminosalicylic acid,
5-(4-nitrobenzenesulfonyl)aminosalicylic acid,
5-[2-(4-nitrophenyl)ethyl]aminosalicylic acid,
5-[3-(4-nitrophenyl)-n-propyl]aminosalicylic acid, and
2-hydroxy-5-(2-(4-trifluoromethyl-phenyl)ethylamino)-benzoic acid.

6. The method of claim 5, wherein the benzylaminosalicylic acid derivative is 2-hydroxy-5-(2-(4-trifluoromethyl-phenyl)ethylamino)-benzoic acid.

7. The method of claim 4, wherein the tetrafluorobenzyl derivative is at least one selected from:
2-hydroxy-5-(2,3,5,6-tetrafluoro-4-trifluoromethyl-benzylamino)-benzoic acid,
2-nitro-5-(2,3,5,6-tetrafluoro-4-trifluoromethyl-benzylamino)-benzoic acid,
2-chloro-5-(2,3,5,6-tetrafluoro-4-trifluoromethyl-benzylamino)-benzoic acid,
2-bromo-5-(2,3,5,6-tetrafluoro-4-trifluoromethyl-benzylamino)-benzoic acid,
2-hydroxy-5-(2,3,5,6-tetrafluoro-4-methyl-benzylamino)-benzoic acid,
2-methyl-5-(2,3,5,6-tetrafluoro-4-trifluoromethyl-benzylamino)-benzoic acid,
2-methoxy-5-(2,3,5,6-tetrafluoro-4-trifluoromethyl-benzylamino)-benzoic acid,
5-(2,3,5,6-tetrafluoro-4-trifluoromethyl-benzylamino)-2-trifluoromethoxy benzoic acid,
2-nitro-4-(2,3,5,6-tetrafluoro-4-trifluoromethyl-benzylamino)phenol,
2-chloro-4-(2,3,5,6-tetrafluoro-4-trifluoromethyl-benzylamino)-phenol,
2-hydroxy-5-(2,3,5,6-tetrafluoro-4-trifluoromethyl-benzylamino)-benzamide,
2-hydroxy-5-(2,3,5,6-tetrafluoro-4-trifluoromethyl-benzylamino)-benzenesulfonic acid,
methyl 2-hydroxy-5-(2,3,5,6-tetrafluoro-4-trifluoromethyl-benzylamino)-benzoate,
2-ethanoyloxy-5-(2,3,5,6-tetrafluoro-4-trifluoromethyl-benzylamino)-benzoic acid,
2-propanoyloxy-5-(2,3,5,6-tetrafluoro-4-trifluoromethyl-benzylamino)-benzoic acid, and
2-cyclohexan carbonyloxy-5-(2,3,5,6-tetrafluoro-4-trifluoromethyl-benzylamino)-benzoic acid.

8. The method of claim 7, wherein the tetrafluorobenzyl derivative is 2-hydroxy-5-(2,3,5,6-tetrafluoro-4-trifluoromethyl-benzylamino)-benzoic acid.

9. A pharmaceutical formulation for treating neuronal death in neurological disease or ocular disease in a human or animal, which comprises a therapeutically effective amount of a cell necrosis inhibitor and a therapeutically effective amount of lithium or a pharmaceutical acceptable salt thereof.

10. The pharmaceutical formulation of claim 9, wherein the neurological disease is selected from amyotrophic lateral sclerosis (ALS, Lou Gehrig's disease), spinal muscular atrophy, Alzheimer's disease, Parkinson's disease, Huntington's disease, stroke, traumatic brain injury, and spinal cord injury.

11. The pharmaceutical formulation of claim 9, wherein the ocular disease is selected from glaucoma, diabetic retinopathy and macular degeneration.

12. The pharmaceutical formulation of claim 9, wherein the cell necrosis inhibitor is at least one selected from:
(i) a benzylaminosalicylic acid derivative of the following formula (I) or pharmaceutically acceptable salts thereof and
(ii) a tetrafluorobenzyl derivative of the following formula (II) or pharmaceutically acceptable salts thereof: 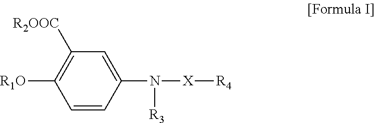
wherein,
X is CO, SO₂ or (CH₂)ₙ, wherein n is an integer from 1 to 5;
R₁ is hydrogen, alkyl or alkanoyl;
R₂ is hydrogen or alkyl;
R₃ is hydrogen or an acetoxy group; and
R₄ is a phenyl group which is unsubstituted or substituted with one or more of nitro, halogen, haloalkyl, and C₁-C₅ alkoxy; 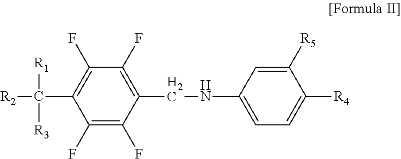
wherein,
R₁, R₂ and R₃ are independently hydrogen or halogen;
R₄ is hydroxy, alkyl, alkoxy, halogen, alkoxy substituted with halogen, alkanoyloxy or nitro; and
R₅ is carboxyl acid, ester having C₁-C₄ alkyl, carboxyamide, sulfonic acid, halogen or nitro.

13. The pharmaceutical formulation of claim 12, wherein the benzylaminosalicylic acid derivative is at least one selected from:
5-benzylaminosalicylic acid,
5-(4-nitrobenzyl)aminosalicylic acid,
5-(4-chlorobenzyl)aminosalicylic acid,
5-(4-trifluoromethylbenzyl)aminosalicylic acid,
5-(4-fluorobenzyl)aminosalicylic acid,
5-(4-methoxybenzyl)aminosalicylic acid,
5-(4-pentafluorobenzyl)aminosalicylic acid,
5-(4-nitrobenzyl)amino-2-hydroxy ethyl benzoate,
5-(4-nitrobenzyl)-N-acetylamino-2-hydroxy ethylbenzoate,
5-(4-nitrobenzyl)-N-acetylamino-2-acetoxy ethylbenzoate,
5-(4-nitrobenzoyl)aminosalicylic acid,
5-(4-nitrobenzenesulfonyl)aminosalicylic acid,
5-[2-(4-nitrophenyl)ethyl]aminosalicylic acid,
5-[3-(4-nitrophenyl)-n-propyl]aminosalicylic acid, and
2-hydroxy-5-(2-(4-trifluoromethyl-phenyl)ethylamino)-benzoic acid.

14. The pharmaceutical formulation of claim 13, wherein the benzylaminosalicylic acid derivative is 2-hydroxy-5-(2-(4-trifluoromethyl-phenyl)ethylamino)-benzoic acid.

15. The pharmaceutical formulation of claim 12, wherein the tetrafluorobenzyl derivative is at least one selected from:
2-hydroxy-5-(2,3,5,6-tetrafluoro-4-trifluoromethyl-benzylamino)-benzoic acid,
2-nitro-5-(2,3,5,6-tetrafluoro-4-trifluoromethyl-benzylamino)-benzoic acid,
2-chloro-5-(2,3,5,6-tetrafluoro-4-trifluoromethyl-benzylamino)-benzoic acid,
2-bromo-5-(2,3,5,6-tetrafluoro-4-trifluoromethyl-benzylamino)-benzoic acid,
2-hydroxy-5-(2,3,5,6-tetrafluoro-4-methyl-benzylamino)-benzoic acid,
2-methyl-5-(2,3,5,6-tetrafluoro-4-trifluoromethyl-benzylamino)-benzoic acid,
2-methoxy-5-(2,3,5,6-tetrafluoro-4-trifluoromethyl-benzylamino)-benzoic acid,
5-(2,3,5,6-tetrafluoro-4-trifluoromethyl-benzylamino)-2-trifluoromethoxy benzoic acid,
2-nitro-4-(2,3,5,6-tetrafluoro-4-trifluoromethyl-benzylamino)phenol,
2-chloro-4-(2,3,5,6-tetrafluoro-4-trifluoromethyl-benzylamino)-phenol,
2-hydroxy-5-(2,3,5,6-tetrafluoro-4-trifluoromethyl-benzylamino)-benzamide,
2-hydroxy-5-(2,3,5,6-tetrafluoro-4-trifluoromethyl-benzylamino)-benzenesulfonic acid,
methyl 2-hydroxy-5-(2,3,5,6-tetrafluoro-4-trifluoromethyl-benzylamino)-benzoate,
2-ethanoyloxy-5-(2,3,5,6-tetrafluoro-4-trifluoromethyl-benzylamino)-benzoic acid,
2-propanoyloxy-5-(2,3,5,6-tetrafluoro-4-trifluoromethyl-benzylamino)-benzoic acid, and
2-cyclohexan carbonyloxy-5-(2,3,5,6-tetrafluoro-4-trifluoromethyl-benzylamino)-benzoic acid.

16. The pharmaceutical formulation of claim 15, wherein the tetrafluorobenzyl derivatives is 2-hydroxy-5-(2,3,5,6-tetrafluoro-4-trifluoromethyl-benzylamino)-benzoic acid.

17. A kit for treating neuronal death in neurological disease or ocular disease in a human or animal, which comprises a therapeutically effective amount of a cell necrosis inhibitor and a therapeutically effective amount of lithium or a pharmaceutical acceptable salt thereof.

18. The kit of claim 17, wherein the neurological disease is selected from amyotrophic lateral sclerosis (ALS, Lou Gehrig's disease), spinal muscular atrophy, Alzheimer's disease, Parkinson's disease, Huntington's disease, stroke, traumatic brain injury, and spinal cord injury.

19. The kit of claim 17, wherein the ocular disease is selected from glaucoma, diabetic retinopathy and macular degeneration.

20. The kit of claim 17, wherein the cell necrosis inhibitor is at least one selected from:
(i) a benzylaminosalicylic acid derivative of the following formula (I) or pharmaceutically acceptable salts thereof and
(ii) a tetrafluorobenzyl derivative of the following formula (II) or pharmaceutically acceptable salts thereof: 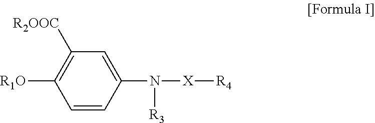
wherein,
X is CO, SO₂ or (CH₂)ₙ, wherein n is an integer from 1 to 5;
R₁ is hydrogen, alkyl or alkanoyl;
R₂ is hydrogen or alkyl;
R₃ is hydrogen or an acetoxy group; and
R₄ is a phenyl group which is unsubstituted or substituted with one or more of nitro, halogen, haloalkyl, and C₁-C₅ alkoxy; 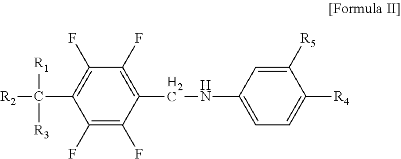
wherein,
R₁, R₂ and R₃ are independently hydrogen or halogen;
R₄ is hydroxy, alkyl, alkoxy, halogen, alkoxy substituted with halogen, alkanoyloxy or nitro; and
R₅ is carboxyl acid, ester having C₁-C₄ alkyl, carboxyamide, sulfonic acid, halogen or nitro.

21. The kit of claim 20, wherein the benzylaminosalicylic acid derivative is at least one selected from:
5-benzylaminosalicylic acid,
5-(4-nitrobenzyl)aminosalicylic acid,
5-(4-chlorobenzyl)aminosalicylic acid,
5-(4-trifluoromethylbenzyl)aminosalicylic acid,
5-(4-fluorobenzyl)aminosalicylic acid,
5-(4-methoxybenzyl)aminosalicylic acid,
5-(4-pentafluorobenzyl)aminosalicylic acid,
5-(4-nitrobenzyl)amino-2-hydroxy ethyl benzoate,
5-(4-nitrobenzyl)-N-acetylamino-2-hydroxy ethylbenzoate,
5-(4-nitrobenzyl)-N-acetylamino-2-acetoxy ethylbenzoate,
5-(4-nitrobenzoyl)aminosalicylic acid,
5-(4-nitrobenzenesulfonyl)aminosalicylic acid,
5-[2-(4-nitrophenyl)ethyl]aminosalicylic acid,
5-[3-(4-nitrophenyl)-n-propyl]aminosalicylic acid, and
2-hydroxy-5-(2-(4-trifluoromethyl-phenyl)ethylamino)-benzoic acid.

22. The kit of claim 21, wherein the benzylaminosalicylic acid derivative is 2-hydroxy-5-(2-(4-trifluoromethyl-phenyl)ethylamino)-benzoic acid.

23. The kit of claim 20, wherein the tetrafluorobenzyl derivative is at least one selected from:
2-hydroxy-5-(2,3,5,6-tetrafluoro-4-trifluoromethyl-benzylamino)-benzoic acid,
2-nitro-5-(2,3,5,6-tetrafluoro-4-trifluoromethyl-benzylamino)-benzoic acid,
2-chloro-5-(2,3,5,6-tetrafluoro-4-trifluoromethyl-benzylamino)-benzoic acid,
2-bromo-5-(2,3,5,6-tetrafluoro-4-trifluoromethyl-benzylamino)-benzoic acid,
2-hydroxy-5-(2,3,5,6-tetrafluoro-4-methyl-benzylamino)-benzoic acid,
2-methyl-5-(2,3,5,6-tetrafluoro-4-trifluoromethyl-benzylamino)-benzoic acid,
2-methoxy-5-(2,3,5,6-tetrafluoro-4-trifluoromethyl-benzylamino)-benzoic acid,
5-(2,3,5,6-tetrafluoro-4-trifluoromethyl-benzylamino)-2-trifluoromethoxy benzoic acid,
2-nitro-4-(2,3,5,6-tetrafluoro-4-trifluoromethyl-benzylamino)phenol,
2-chloro-4-(2,3,5,6-tetrafluoro-4-trifluoromethyl-benzylamino)-phenol,
2-hydroxy-5-(2,3,5,6-tetrafluoro-4-trifluoromethyl-benzylamino)-benzamide,
2-hydroxy-5-(2,3,5,6-tetrafluoro-4-trifluoromethyl-benzylamino)-benzenesulfonic acid,
methyl 2-hydroxy-5-(2,3,5,6-tetrafluoro-4-trifluoromethyl-benzylamino)-benzoate,
2-ethanoyloxy-5-(2,3,5,6-tetrafluoro-4-trifluoromethyl-benzylamino)-benzoic acid,
2-propanoyloxy-5-(2,3,5,6-tetrafluoro-4-trifluoromethyl-benzylamino)-benzoic acid, and
2-cyclohexan carbonyloxy-5-(2,3,5,6-tetrafluoro-4-trifluoromethyl-benzylamino)-benzoic acid.

24. The kit of claim 23, wherein the tetrafluorobenzyl derivative is 2-hydroxy-5-(2,3,5,6-tetrafluoro-4-trifluoromethyl-benzylamino)-benzoic acid.
